(19) United States
(12) Patent Application Publication
(10) Pub. No.: US 2024/0218083 A1
(43) Pub. Date: Jul. 4, 2024

(54) MOLECULES WITH ENGINEERED ANTIBODY CONSTANT REGION VARIANTS

(71) Applicant: Janssen Biotech, Inc., Horsham, PA (US)

(72) Inventors: Sanjaya SINGH, Blue Bell, PA (US); Ellen CHI, San Diego, CA (US); Martin CASE, San Diego, CA (US); Hong Mimi ZHOU, San Diego, CA (US); Linus HYUN, San Diego, CA (US); Jennifer FURMAN, San Diego, CA (US); Ann LACOMBE, San Diego, CA (US)

(21) Appl. No.: 18/555,924

(22) PCT Filed: Apr. 18, 2022
(86) PCT No.: PCT/US22/25186

§ 371 Date: Oct. 18, 2023

Related U.S. Application Data
(60) Provisional application No. 63/176,736, filed on Apr. 19, 2021.
(60) Provisional application No. 63/176,725, filed on Apr. 19, 2021.
(60) Provisional application No. 63/176,731, filed on Apr. 19, 2021.
(60) Provisional application No. 63/176,720, filed on Apr. 19, 2021.
(60) Provisional application No. 63/176,718, filed on Apr. 19, 2021.

(51) Int. Cl. C07K 16/46; A61K 39/00; C40B 40/10

(52) U.S. Cl. CPC C07K 16/468; C40B 40/10; A61K 2039/505; C07K 2317/515; C07K 2317/522; C07K 2317/55

(57) ABSTRACT

A binding molecule comprising (i) a first polypeptide comprising a heavy chain variable region (VH) and a region derived from the first constant region of an antibody heavy chain (CH1), and (ii) a second polypeptide comprising a light chain variable region (VL) and a region derived from the constant region of an antibody light chain (CL), wherein the region derived from the CH1 region and/or the region derived from the CL region comprises one or more antigen binding loop(s).

### CROSS-REFERENCE TO RELATED APPLICATIONS

This application claims the benefit of U.S. Ser. No. 63/176,718, filed Apr. 19, 2021; U.S. Ser. No. 63/176,720, filed Apr. 19, 2021; U.S. Ser. No. 63/176,725, filed Apr. 19, 2021; U.S. Ser. No. 63/176,731, filed Apr. 19, 2021; U.S. Ser. No. 63/176,736, filed Apr. 19, 2021, each of which is herein incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14620-683-228_SEQ_LISTING.txt” and a creation date of Apr. 14, 2022 and having a size of 18,124 bytes. The sequence listing submitted via EFS-Web is part of the specification and is herein incorporated by reference in its entirety.

### 1. FIELD

Provided herein are binding molecules comprising variants of an antibody constant region capable of binding to an antigen. Also provided herein are pharmaceutical compositions or kits comprising the binding molecules, processes of making, and methods of using the same.

### 2. BACKGROUND

The present disclosure is the first to develop improved antibody structures with additional antigen binding sites outside of the VH and VL regions and uses thereof. Antibodies have been successfully used for treating various diseases and disorders. A conventional antibody recognizes its antigen via the VH region and the VL region. Various techniques have been developed to engineer a protein to bind to a target it does not normally bind to. To expand conventional antibodies' binding capacity to multiple antigens and thus to increase antibodies' therapeutic effect, there is a need in the art of improved antibody structures with additional antigen binding sites outside of the VH and VL regions.

### 3. SUMMARY

In one aspect, provided herein is a binding molecule comprising a region derived from a CH1 region of an antibody heavy chain and/or a region derived from a CL region of an antibody light chain, wherein the region derived from the CH1 region and/or the region derived from the CL region comprises one or more antigen binding loop(s).

In one aspect, provided herein is a binding molecule comprising: (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the region derived from the CH1 region and/or the region derived from the CL region comprises one or more antigen binding loop(s).

In some embodiments, the one or more antigen binding loop(s) in the region derived from the CH1 region are at the AB, BC, CD, DE, EF, and/or FG loop regions of the CH1 region. In some embodiments, the one or more antigen binding loop(s) in the region derived from the CL region are at the AB, BC, CD, DE, EF, and/or FG loop regions of the CL region. In some embodiments, the one or more antigen binding loop(s) in the region derived from the CH1 region are located outside of AB, BC, CD, DE, EF, and/or FG loop regions of the CH1 region. In some embodiments, the one or more antigen binding loop(s) in the region derived from the CL region are located outside of AB, BC, CD, DE, EF, and/or FG loop regions of the CL region. In some embodiments, the one or more antigen binding loop(s) in the region derived from the CH1 region are located in A, B, C, D, E, and/or F β-strands of the CH1 region. In some embodiments, the one or more antigen binding loop(s) in the region derived from the CL region are located in A, B, C, D, E, and/or F β-strands of the CL region.

In some embodiments, the region derived from the CH1 region comprises one or two antigen binding loop(s). In some embodiments, the region derived from the CL region comprises one or two antigen binding loop(s).

In some embodiments, the region derived from the CH1 region comprises one antigen binding loop at the CD loop region of the CH1 region. In some embodiments, the region derived from the CH1 region comprises one antigen binding loop at the DE loop region of the CH1 region. In some embodiments, the region derived from the CH1 region comprises one antigen binding loop at the CD loop region of the CH1 region and one antigen binding loop at the DE loop region of the CH1 region.

In some embodiments, the region derived from the CL region comprises one antigen binding loop at the CD loop region of the CL region. In some embodiments, the region derived from the CL region comprises one antigen binding loop at the DE loop region of the CL region. In some embodiments, the region derived from the CL region comprises one antigen binding loop at the CD loop region of the CL region and one antigen binding loop at the DE loop region of the CL region.

In some embodiments, the region derived from the CH1 region comprises one antigen binding loop at the CD loop region of the CH1 region; and the region derived from the CL region comprises one antigen binding loop at the CD loop region of the CL region. In some embodiments, the region derived from the CH1 region comprises one antigen binding loop at the CD loop region of the CH1 region; and the region derived from the CL region comprises one antigen binding loop at the DE loop region of the CL region. In some embodiments, the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region.

In some embodiments, the region derived from the CH1 region comprises one antigen binding loop at the DE loop region of the CH1 region; and the region derived from the CL region comprises one antigen binding loop at the CD loop region of the CL region. In some embodiments, the region derived from the CH1 region comprises one antigen binding loop at the DE loop region of the CH1 region; and the region derived from the CL region comprises one antigen binding loop at the DE loop region of the CL region. In some embodiments, the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region.

In some embodiments, the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region. In some embodiments, the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region. In some embodiments, the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.

In some embodiments, the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1. In some embodiments, the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2. In some embodiments, the region derived from the CL region is a region derived from a human CL lambda region comprising an amino acid sequence of SEQ ID NO:3, and wherein the region derived from the CL region comprises an amino acid sequence having at least 70%, 75%, 80%, 85%, 90% or 95% identity to SEQ ID NO:3.

In some embodiments, the antigen binding loop at the CD loop region of the CH1 region replaces the amino acid residues TSG of the CD loop of the human IgG1 CH1 region. In some embodiments, the antigen binding loop at the DE loop region of the CH1 region replaces the amino acid residues QSS of the DE loop of the human IgG1 CH1 region. In some embodiments, the antigen binding loop at the CD loop region of the CL region replaces the amino acid residues SGNS of the CD loop of the human CL kappa region. In some embodiments, the antigen binding loop at the DE loop region of the CL region replaces the amino acid residues SKD of the DE loop of the human CL kappa region.

In some embodiments, each of the one or more antigen binding loop(s) comprises 7 to 15 amino acid residues.

In some embodiments, the VH region and the VL region bind to an first antigen; and the region derived from the CH1 region and/or the region derived from the CL region bind to a second antigen. In some embodiments, the first antigen and the second antigen are the same antigen. In some embodiments, the first antigen and the second antigen are two different antigens.

In another aspect, provided herein is a nucleic acid encoding the binding molecule.

In another aspect, provided herein is a vector comprising the nucleic acid encoding the binding molecule.

In yet one aspect, provided herein is a method of making a binding molecule, comprising expressing a polynucleotide encoding a binding molecule in a host cell. In some embodiments, the binding molecule comprises a region derived from a CH1 region of an antibody heavy chain and/or a region derived from a CL region of an antibody light chain, wherein the region derived from the CH1 region and/or the region derived from the CL region comprises one or more antigen binding loop(s). In some embodiments, the binding molecule comprises: (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the region derived from the CH1 region and/or the region derived from the CL region comprises one or more antigen binding loop(s).

In yet one aspect, provided herein is a pharmaceutical composition. In some embodiments, the pharmaceutical composition comprises (a) a binding molecule comprising a region derived from a CH1 region of an antibody heavy chain and/or a region derived from a CL region of an antibody light chain, wherein the region derived from the CH1 region and/or the region derived from the CL region comprises one or more antigen binding loop(s); and (b) a pharmaceutically acceptable excipient. In some embodiments, the pharmaceutical composition comprises a binding molecule comprising: (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the region derived from the CH1 region and/or the region derived from the CL region comprises one or more antigen binding loop(s); and (b) a pharmaceutically acceptable excipient.

In yet one aspect, provided herein is a method of treating a disease or disorder in a subject, comprising administering to the subject a binding molecule or a nucleic acid encoding a binding molecule. In some embodiments, the binding molecule comprises a region derived from a CH1 region of an antibody heavy chain and/or a region derived from a CL region of an antibody light chain, wherein the region derived from the CH1 region and/or the region derived from the CL region comprises one or more antigen binding loop(s). In some embodiments, the binding molecule comprises: (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the region derived from the CH1 region and/or the region derived from the CL region comprises one or more antigen binding loop(s). In some embodiments, the disease or disorder is associated with the antigens of the binding molecule.

In one aspect, provided herein is a constant region library (CRL) comprising a population of binding molecules, wherein each of the binding molecules comprises (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the population of the binding molecules comprise diverse amino acid sequences in the region derived from the CH1 region and/or the region derived from the CL region. In another aspect, provided herein is a constant region library (CRL) comprising a population of molecules each comprising a region derived from a CH1 region and/or a region derived from a CL region of an antibody, wherein the population of the molecules comprise diverse amino acid sequences in the region derived from the CH1 region and/or the region derived from the CL region.

In some embodiments, the diverse amino acid sequences in the region derived from the CH1 region are at the AB, BC, CD, DE, EF, and/or FG loop regions of the CH1 region. In some embodiments, the diverse amino acid sequences in the region derived from the CL region are at the AB, BC, CD, DE, EF, and/or FG loop regions of the CL region. In some embodiments, the diverse amino acid sequences in the region derived from the CH1 region are located outside of AB, BC, CD, DE, EF, and/or FG loop regions of the CH1 region. In some embodiments, the diverse amino acid sequences in the region derived from the CL region are located outside of AB, BC, CD, DE, EF, and/or FG loop regions of the CL region. In some embodiments, the diverse amino acid sequences in the region derived from the CH1 region are located in A, B, C, D, E, and/or F β-strands of the CH1 region. In some embodiments, the diverse amino acid sequences in the region derived from the CL region are located in A, B, C, D, E, and/or F β-strands of the CL region.

In some embodiments, the population of the molecules comprise diverse amino acid sequences in one or two loop region(s) in the region derived from the CH1 region. In some embodiments, the population of the molecules comprise diverse amino acid sequences in one or two loop region(s) in the region derived from the CL region.

In some embodiments, the population of the molecules comprise diverse amino acid sequences at the CD loop region of the CH1 region. In some embodiments, the population of the molecules comprise diverse amino acid sequences at the DE loop region of the CH1 region. In some embodiments, the population of the molecules comprise diverse amino acid sequences at the CD loop region and the DE loop region of the CH1 region.

In some embodiments, the population of the molecules comprise diverse amino acid sequences at the CD loop region of the CL region. In some embodiments, the population of the molecules comprise diverse amino acid sequences at the DE loop region of the CL region. In some embodiments, the population of the molecules comprise diverse amino acid sequences at the CD loop region and the DE loop region of the CL region.

In some embodiments, the population of the molecules comprise diverse amino acid sequences at the CD loop region of the CH1 region; and the population of the molecules comprise diverse amino acid sequences at the CD loop region of the CL region. In some embodiments, the population of the molecules comprise diverse amino acid sequences at the CD loop region of the CH1 region; and the population of the molecules comprise diverse amino acid sequences at the DE loop region of the CL region. In some embodiments, the population of the molecules comprise diverse amino acid sequences at the CD loop region of the CH1 region; and the population of the molecules comprise diverse amino acid sequences at the CD loop region and the DE loop region of the CL region.

In some embodiments, the population of the molecules comprise diverse amino acid sequences at the DE loop region of the CH1 region; and the population of the molecules comprise diverse amino acid sequences at the CD loop region of the CL region. In some embodiments, the population of the molecules comprise diverse amino acid sequences at the DE loop region of the CH1 region; and the population of the molecules comprise diverse amino acid sequences at the DE loop region of the CL region. In some embodiments, the population of the molecules comprise diverse amino acid sequences at the DE loop region of the CH1 region; and the population of the molecules comprise diverse amino acid sequences at the CD loop region and the DE loop region of the CL region.

In some embodiments, the population of the molecules comprise diverse amino acid sequences at the CD loop region of the CH1 region and the DE loop region of the CH1 region; and the population of the molecules comprise diverse amino acid sequences at the CD loop region of the CL region. In some embodiments, the population of the molecules comprise diverse amino acid sequences at the CD loop region and the DE loop region of the CH1 region; and the population of the molecules comprise diverse amino acid sequences at the DE loop region of the CL region. In some embodiments, the population of the molecules comprise diverse amino acid sequences at the CD loop region and the DE loop region of the CH1 region; and the population of the molecules comprise diverse amino acid sequences at the CD loop region and the DE loop region of the CL region.

In some embodiments, the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1. In some embodiments, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2. In some embodiments, wherein the region derived from the CL region is a region derived from a human CL lambda region comprising an amino acid sequence of SEQ ID NO:3, and wherein the region derived from the CL region comprises an amino acid sequence having at least 70%, 75%, 80%, 85%, 90% or 95% identity to SEQ ID NO:3.

In some embodiments, the amino acid residues TSG of the CD loop of the human IgG1 CH1 region are replaced with diverse amino acid sequences in the molecules in the CRL. In some embodiments, the amino acid residues QSS of the DE loop of the human IgG1 CH1 region are replaced with diverse amino acid sequences in the molecules in the CRL. In some embodiments, the amino acid residues SGNS of the CD loop of the human CL kappa region are replaced with diverse amino acid sequences in the molecules in the CRL. In some embodiments, the amino acid residues SKD of the DE loop of the human CL kappa region are replaced with diverse amino acid sequences in the molecules in the CRL.

In some embodiments, the diverse amino acid sequences comprise 7 to 15 amino acid residues. In some embodiments, each of the molecules further comprise a VH region and a VL region.

In some embodiments, the binding molecules or the molecules are Fab fragments.

In some embodiments, the diversity of the CRL with one loop region ranges from 10⁷ to 10¹⁶. In some embodiments, the diversity of the CRL with two loop regions ranges from 10¹⁸ to 10³³.

In another aspect, provided herein is a method for identifying a binding molecule comprising a first binding domain that binds to a first antigen and a second binding domain that binds to a second antigen, comprising screening the CRL for identifying the binding molecule that binds to the second antigen with a higher affinity than a reference level, wherein the first binding domain comprises the VH region and the VL region of an antibody, and wherein the second binding domain comprises an antibody constant region variant. In another aspect, provided herein is a method of producing a binding molecule comprising a first step for performing a function of identifying an antibody constant region variant capable of binding to an antigen; and a second step of constructing the binding molecule that comprises the antibody constant region variant. In some embodiments, the first step comprising screening the CRL. In yet another aspect, provided herein a binding molecule produced according to the methods disclosed herein.

### 4. BRIEF DESCRIPTION OF THE FIGURES

FIG. 1A shows an exemplary binding molecule comprising constant region variants provided herein. FIGS. 1B and 1C depict the construction of exemplary Fab Constant Region Libraries (CRLs). FIG. 1B shows the position and original sequence of CD and DE loops located within human CL kappa and human IgG1 CH1. One or more of the highlighted positions in FIG. 1B are replaced with diversified libraries used to select novel binding molecules against a target of interest. FIG. 1C shows the varied compositions and theoretical diversities of binding loops of Fab CRLs.

FIGS. 2A, 2B, and 2C depicts the selection of Fab constant region binders to anti-polyhistidine monoclonal antibody. Specifically, FIG. 2A depicts the result of polyclonal phage ELISA for anti-polyhistidine monoclonal antibody binding of three enriched pools after one to six rounds of panning; FIG. 2B depicts the result of polyclonal phage ELISA for XO1B1 binding of three enriched pools after one to six rounds of panning; FIG. 2C depicts the amino acid sequences of the binding loops of nine clones after four rounds of panning.

FIG. 3 depicts the overall process of selecting Fab constant region binders to mEphA2-Fc from Fab CRLs.

FIG. 4A depicts the result of polyclonal phage ELISA for mEphA2-Fc binding of five enriched pools after four to eight rounds of panning. The positive control for mEphA2-Fc binding is an anti-mEphA2-Fc CH2 domain-phage fusion, and the negative control is the anti-XO1B1 parent Fab-phage fusion. Signal relative to the parent Fab is calculated as: (mEphA2-Fc RLU of the panning pool)/(mEphA2-Fc RLU of the parent Fab). FIGS. 4B and 4C depict the result of monoclonal phage ELISA for mEphA2-Fc binding (FIG. 4B) and XO1B1 binding (FIG. 4C) of 378 clones from the five enriched pools after six to eight rounds of panning. FIG. 4D depicts the result of monoclonal phage ELISA for both mEphA2-Fc binding and XO1B1 binding of sixteen clones that are selected for further analysis by Sanger sequencing, all of which are from P8 after six rounds of panning. FIG. 4E depicts the single amino acid sequence derived from the CH1 CD loop library that was identified from all sixteen clones selected from P8.

FIG. 4F depicts the size exclusion-high performance liquid chromatography (SE-HPLC) and reducing (R) and non-reducing (NR) sodium dodecyl sulfate-polyacrylamide gel electrophoresis (SDS-PAGE) characterization of the single Fab constant region binder to mEphA2-Fc (identified as EPAXB1) isolated from mammalian expression in HEK Expi293 cells using a single-step Immobilized Metal Affinity Chromatography (IMAC) purification. FIG. 4F also depicts the determination of molecular weight of EPAXB1 using mass spectrometry. FIG. 4G depicts the binding kinetics and affinity of EPAXB1 against both mEphA2-Fc and XO1B1 using surface plasmon resonance (SPR) analysis. FIG. 4G also depicts the binding kinetics and affinity of the anti-XO1B1 parent Fab that lacks engineered CRLs against both mEphA2-Fc and XO1B1.

FIG. 5 depicts new fab constant region binders (EPAXB17, EPAXB27, and EPAXB28) identified from a subset of the panning pools through NGS and SPR.

FIG. 6A depicts the pairing of the constant region of EPAXB1 with new variable regions and the reformatting of EPAXB1 as monoclonal antibody. FIG. 6B depicts the size exclusion-high performance liquid chromatography (SE-HPLC) characterization of the reformatted purified Fabs and monoclonal antibody. FIG. 6C depicts that the reformatted bispecific Fabs and monoclonal antibody retain binding to their corresponding targets.

FIG. 7 depicts purification yields observed by SE-HPLC for anti-IL23R×anti-EphA2 and anti-HER2×anti-EphA2 Fabs reformatted into standard monoclonal antibody formats.

FIGS. 8A-8D depict binding data for HER2×EphA2 and IL23×EphA2 bispecific antibodies and corresponding monospecific control antibodies to untransfected HEK cells (FIG. 8A), HEK cells stably expressing human HER2 (FIG. 8B), HEK cells stably expressing human EphA2 (FIG. 8C), and HEK cells stably expressing human IL23R (FIG. 8D).

FIGS. 9A-9C depict simultaneous binding of anti-HER2×anti-EphA2 and anti-IL23R×anti-EphA2 bispecific Fabs (FIGS. 9A and 9B, respectively) and anti-HER2×anti-EphA2 bispecific in mAb format (FIG. 9C) to their respective targets by biolayer interferometry (BLI).

### 5. DETAILED DESCRIPTION

The present disclosure is based, in part, on the surprising finding that the CH1 and/or CL region of a Fab can be modified so that the Fab can bind to a desired second antigen. Such modifications are achieved by replacing certain amino acids that are originally present at the surface of the CH1 and/or CL region with diversified amino acids that can be selected to bind to a target of interest. Such modifications are also achieved by introducing additional diversified amino acids that can be selected to bind to a target of interest at the surface of the CH1 and/or CL regions.

### 5.1 Definitions

Techniques and procedures described or referenced herein include those that are generally well understood and/or commonly employed using conventional methodology by those skilled in the art, such as, for example, the widely utilized methodologies described in Molecular Cloning: A Laboratory Manual (Sambrook, et al., 3d ed. 2001); Current Protocols in Molecular Biology (Ausubel, et al. eds., 2003); Therapeutic Monoclonal Antibodies: From Bench to Clinic (An, ed. 2009); Monoclonal Antibodies: Methods and Protocols (Albitar, ed. 2010); and Antibody Engineering Vols 1 and 2 (Kontermann and Dübel, eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity), formed from at least two intact antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, and anti-idiotypic (anti-Id) antibodies. The term “antibody” as used herein also comprises any binding molecule having a Fc region and a functional fragment (e.g., an antigen-binding fragment) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers, ed., 1995); Huston, et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

An “antigen” is a structure to which an antibody can selectively bind. A target antigen may be a polypeptide, carbohydrate, nucleic acid, lipid, hapten, or other naturally occurring or synthetic compound. In some embodiments of each or any of the above- or below-mentioned embodiments, the target antigen is a polypeptide. In certain embodiments, an antigen is associated with a cell, for example, is present on or in a cell.

An “intact” antibody is one comprising an antigen binding site as well as a constant domain (CL) and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody described herein, the terms such as “bind to,” “that specifically bind to,” and analogous terms are also used interchangeably herein and refer to antibodies of antigen binding domains that specifically bind to an antigen, such as a polypeptide. An antibody or antigen binding domain that binds to or specifically binds to an antigen may be cross-reactive with related antigens. In certain embodiments, an antibody or antigen binding domain that binds to or specifically binds to an antigen does not cross-react with other antigens. An antibody or antigen binding domain that binds to or specifically binds to an antigen can be identified, for example, by immunoassays, Octet®, Biacore®, or other techniques known to those of skill in the art. In some embodiments of each or any of the above- or below-mentioned embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul, ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. With regard to terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding domain that binds to an antigen includes one that is capable of binding the antigen with sufficient affinity such that the antibody is useful, for example, as a diagnostic or therapeutic agent in targeting the antigen. In certain embodiments, an antibody or antigen binding domain that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments of each or any of the above- or below-mentioned embodiments, an antibody or antigen binding domain that binds to an antigen has a dissociation constant (K_{D}) of 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM or any K_{D} in between a range defined by any two aforementioned K_{D} values. In certain embodiments, an antibody or antigen binding domain binds to an epitope of an antigen that is conserved among the antigen from different species (e.g., between human and cynomolgus macaque species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen, et al., J. Mol Biol, 1999, 293:865-81). The K_{D} or K_{D} value may also be measured by using biolayer interferometry (BLI) or surface plasmon resonance (SPR) assays by Octet®, using, for example, an Octet® Red96 system, or an Octet® Red384 system, or by Biacore®, using, for example, a Biacore® 2000 or a Biacore® 3000. An “on-rate” or “rate of association” or “association rate” or “kₒₙ” may also be determined with the same biolayer interferometry (BLI) or surface plasmon resonance (SPR) techniques described above using, for example, the Octet® Red96, the Biacore® 2000, the Biacore® 3000 system, or the Biacore® 8000 system.

In certain embodiments, the antibodies can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (see U.S. Pat. No. 4,816,567; and Morrison, et al., Proc. Natl. Acad. Sci. USA, 1984, 81:6851-55).

In certain embodiments, the antibodies can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones, et al., Nature, 1986, 321:522-25; Riechmann, et al., Nature, 1988, 332:323-29; Presta, Curr. Op. Struct. Biol., 1992, 2:593-96; Carter, et al., Proc. Natl. Acad. Sci. USA, 1992, 89:4285-89; U.S. Pat. Nos. 6,800,738; 6,719,971; 6,639,055; 6,407,213; and 6,054,297.

In certain embodiments, the antibodies can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat, et al. (see Kabat, et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 1991, 227:381; Marks, et al., 1991, J. Mol. Biol., 1991, 222:581) and yeast display libraries (Chao, et al., Nature Protocols, 2006, 1: 755-68). Also available for the preparation of human monoclonal antibodies are methods described in Cole, et al., Monoclonal Antibodies and Cancer Therapy 77 (1985); Boerner, et al., J. Immunol., 1991, 147(1): 86-95; and van Dijk and van de Winkel, Curr. Opin. Pharmacol., 2001, 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol., 1995, 6(5):561-66; Brüggemann and Taussing, Curr. Opin. Biotechnol., 1997, 8(4):455-58; and U.S. Pat. Nos. 6,075,181 and 6,150,584 regarding XENOMOUSE™ technology). See also, for example, Li, et al., Proc. Natl. Acad. Sci. USA, 2006, 103:3557-62, regarding human antibodies generated via a human B-cell hybridoma technology.

In certain embodiments, the antibodies can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D., et al., Nucl. Acids Res., 1992 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson, et al., Nature, 1991, 352:624-28 and Marks, et al., J. Mol. Biol., 1991, 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites, et al. eds., 8th ed. 1994); and Immunobiology (Janeway, et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5^{th} ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat, et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat, et al., supra). The “EU numbering system” or “EU index” is generally used when referring to a residue in an immunoglobulin heavy chain constant region (e.g., the EU index reported in Kabat, et al., supra). The “EU index as in Kabat” refers to the residue numbering of the human IgG1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel, eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc, et al., Dev. Comp. Immunol., 2003, 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol., 2001, 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc, et al., supra). The residues from each of these hypervariable regions or CDRs are noted below.

TABLE 1

| Loop | Kabat | AbM | Chothia | Contact | IMGT |
|---|---|---|---|---|---|
| CDR L1 | L24--L34 | L24--L34 | L24--L34 | L30--L36 | L27--L38 |
| CDR L2 | L50--L56 | L50--L56 | L50--L56 | L46--L55 | L56--L65 |
| CDR L3 | L89--L97 | L89--L97 | L89--L97 | L89--L96 | L105--L117 |
| CDR H1 | H31--H35B | H26--H35B | H26--H32 . . . 34 | H30--H35B | H27--H38 |
| | (Kabat Numbering) | | | | |
| CDR H1 | H31--H35 | H26--H35 | H26--H32 | H30--H35 | |
| | (Chothia Numbering) | | | | |
| CDR H2 | H50--H65 | H50--H58 | H52--H56 | H47--H58 | H56--H65 |
| CDR H3 | H95--H102 | H95--H102 | H95--H102 | H93--H101 | H105--H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues. There are typically four FR regions in each of VH and VL regions. The FR regions in VH are VH FR1, VH FR2, VH FR3, and VH FR4 (or FR H1, FR H2, FR H3 and FR H4). The FR regions in VL are VL FR1, VL FR2, VL FR3 and VL FR4 (or FR L1, FR L2, FR L3 and FR L4).

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “loop region(s)” refers to structural loops connecting β-strands, which in turn constitute β-sheets, a common motif of the regular protein secondary structure. It is well known to those skilled in the art that β-strands are lettered sequentially (A, B, C, D, E, F, etc.) with respect to the order of their occurrence in the primary amino acid sequence of the protein domain. The structural loops are labelled based on the β-strands they are connecting. For instance, AB loop refers to the structural loop connecting β-strands A and B; BC loop refers to the structural loop connecting β-strands B and C; CD loop refers to the structural loop connecting β-strands C and D; DE loop refers to the structural loop connecting β-strands D and E; etc.

The term “antigen binding loop(s)” refers to a polypeptide region that can specifically bind to an antigen. In some instances, the antigen binding loops are CDRs located within the VH and/or VL region. In other cases, the antigen binding loops are polypeptide located outside of the VH and/or VL region. In some embodiments, the antigen binding loops provided herein are located in the CH1 region. In some embodiments, the antigen binding loops provided herein are located in the CL region. In some embodiments, the antigen binding loops are located in both the CH1 region and the CL region. In some embodiments, the antigen binding loops are located in AB, BC, CD, DE, EF, and/or FG loop regions of the CH1 region. In some embodiments, the antigen binding loops are located in AB, BC, CD, DE, EF, and/or FG loop regions of the CL region. In some embodiments, the antigen binding loops are located outside of AB, BC, CD, DE, EF, and/or FG loop regions of the CH1 region. In some embodiments, the antigen binding loops are located outside of AB, BC, CD, DE, EF, and/or FG loop regions of the CL region. In some embodiments, the antigen binding loops are located in A, B, C, D, E, and/or F β-strands of the CL region. In some embodiments, the antigen binding loops are located in A, B, C, D, E, and/or F β-strands of the CH1 region.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

As used herein, an “epitope” is a term in the art and refers to a localized region of an antigen to which an antibody can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In some embodiments of each or any of the above- or below-mentioned embodiments, an antibody requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding an antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide,” “nucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “multispecific antibody” refers to an antibody that comprises a plurality of antigen binding sites, wherein a first antigen binding site of the plurality has binding specificity for a first epitope and a second antigen binding site of the plurality has binding specificity for a second epitope. In some embodiments of each or any of the above- or below-mentioned embodiments, the first and second epitopes do not overlap or do not substantially overlap. In some embodiments of each or any of the above- or below-mentioned embodiments, the first and second epitopes are on different antigens, e.g., the different proteins (or different subunits of a multimeric protein). In some embodiments of each or any of the above- or below-mentioned embodiments, a multispecific antibody comprises a third, fourth, or fifth antigen binding site. In some embodiments of each or any of the above- or below-mentioned embodiments, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first antigen binding site which has binding specificity for a first epitope and a second antigen binding site that has binding specificity for a second epitope. In some embodiments of each or any of the above- or below-mentioned embodiments, the first and second epitopes are on different antigens, e.g., the different proteins (or different subunits of a multimeric protein). In some embodiments of each or any of the above- or below-mentioned embodiments, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In some embodiments of each or any of the above- or below-mentioned embodiments, a bispecific antibody comprises a half antibody, or fragment thereof, having binding specificity for a first epitope and a half antibody, or fragment thereof, having binding specificity for a second epitope. In an embodiment, a bispecific antibody comprises a heavy chain variable domain sequence and a light chain variable domain sequence which have binding specificity for a first epitope and a region derived from a CH1 region of an antibody heavy chain and/or a region derived from a CL region of an antibody light chain which have binding specificity for a second epitope.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)), or vehicle.

In some embodiments of each or any of the above- or below-mentioned embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In some embodiments of each or any of the above- or below-mentioned embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments of each or any of the above- or below-mentioned embodiments, pharmaceutically acceptable excipients are nontoxic to the cell or mammal being exposed thereto at the dosages and concentrations employed. In some embodiments of each or any of the above- or below-mentioned embodiments, a pharmaceutically acceptable excipient is an aqueous pH buffered solution.

In some embodiments of each or any of the above- or below-mentioned embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antibody, for example, in isolated or purified form, together with a suitable amount of excipients.

The term “effective amount” or “therapeutically effective amount” as used herein refers to the amount of an antibody or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In some embodiments of each or any of the above- or below-mentioned embodiments, the subject is a mammal, e.g., a human, diagnosed with a condition or disorder. In some embodiments of each or any of the above- or below-mentioned embodiments, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Binding Molecules

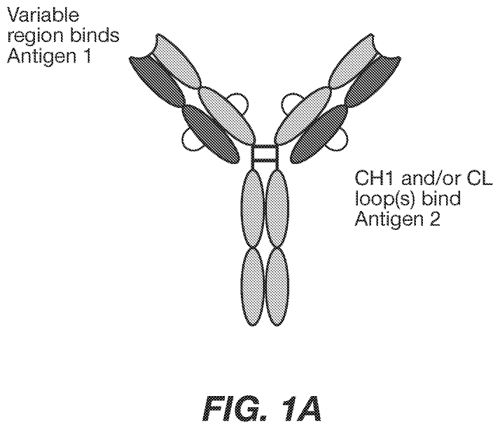

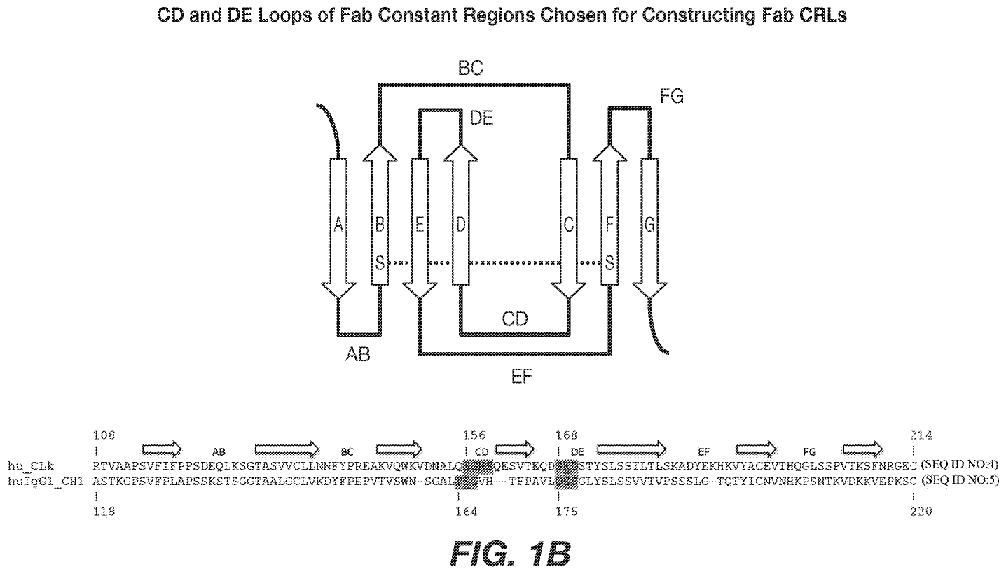

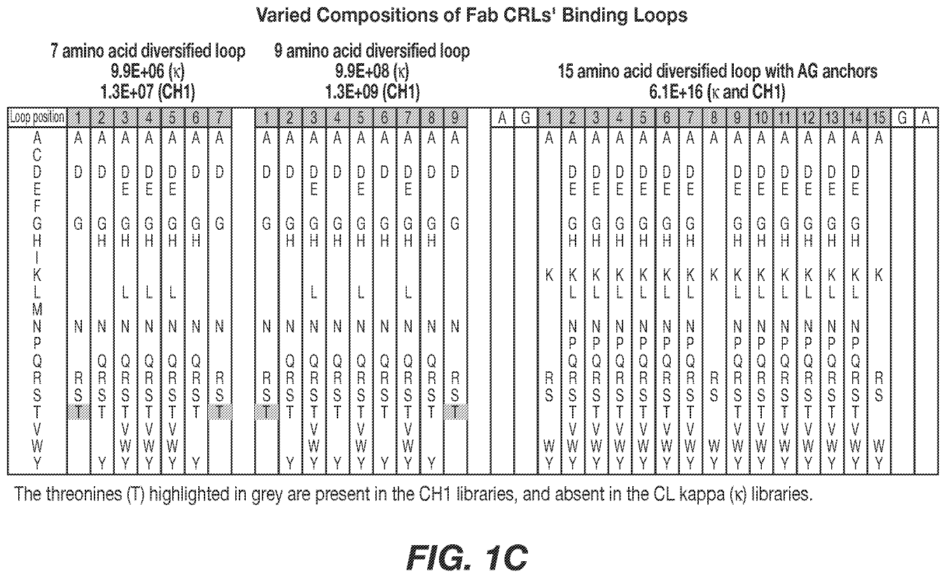

The binding molecules provided herein comprise at least one engineered antibody constant region variant (e.g., a CH1 region variant and/or a CL region variant), wherein the constant region variant comprises one or more antigen binding loops, thus the constant region variant in the present molecule confers antigen binding capability. In some embodiments, the antigen binding loop in the constant region variants provided herein is located in one of loop regions in an antibody constant region (e.g., a CH1 region or a CL region). A “loop region” of an antibody constant region refers to structural loops connecting β-strands (which in turn constitute β-sheets, a common motif of the regular protein secondary structure). It is well known to those skilled in the art that β-strands are lettered sequentially (A, B, C, D, E, F, etc.) with respect to the order of their occurrence in the primary amino acid sequence of the protein domain. The structural loops are labelled based on the β-strands they are connecting. For instance, AB loop refers to the structural loop connecting β-strands A and B; BC loop refers to the structural loop connecting β-strands B and C; CD loop refers to the structural loop connecting β-strands C and D; DE loop refers to the structural loop connecting β-strands D and E; EF loop refers to the structural loop connecting β-strands E and F; FG loop refers to the structural loop connecting β-strands F and G. A typical CH1 region and CL region comprises seven β-strands—A, B, C, D, E, F, and G, and six loop regions—AB loop, BC loop, CD loop, DE loop, EF loop, and FG loop (e.g., as shown in FIG. 1B).

In some embodiments, one or more antigen binding loop(s) are introduced to and/or replace amino acid residues within the AB, BC, CD, DE, EF, and/or FG loop regions of the CH1 region. In some embodiments, one or more antigen binding loop(s) are introduced to and/or replace amino acid residues within the AB, BC, CD, DE, EF, and/or FG loop regions of the CL region. In some embodiments, one or more antigen binding loop(s) are introduced to and/or replace amino acid residues outside of the AB, BC, CD, DE, EF, and/or FG loop regions of the CH1 region. In some embodiments, one or more antigen binding loop(s) are introduced to and/or replace amino acid residues outside of the AB, BC, CD, DE, EF, and/or FG loop regions of the CL region. In some embodiments, one or more antigen binding loop(s) are introduced to and/or replace amino acid residues within the A, B, C, D, E, and/or F β-strands regions of the CH1 region. In some embodiments, one or more antigen binding loop(s) are introduced to and/or replace amino acid residues within the A, B, C, D, E, and/or F β-strands regions of the CL region.

In some embodiments, the antigen binding loop provided herein can be inserted in a loop region. An antigen binding loop can be inserted in AB loop region of CH1 or CL region. An antigen binding loop can be inserted in BC loop region of CH1 or CL region. An antigen binding loop can be inserted in CD loop region of CH1 or CL region. An antigen binding loop can be inserted in DE loop region of CH1 or CL region. An antigen binding loop can be inserted in EF loop region of CH1 or CL region. An antigen binding loop can be inserted in FG loop region of CH1 or CL region.

In other embodiments, the antigen binding loop provided herein can replace a region within a loop region. An antigen binding loop can replace a region within AB loop region of CH1 or CL region. An antigen binding loop can replace a region within BC loop region of CH1 or CL region. An antigen binding loop can replace a region within CD loop region of CH1 or CL region. An antigen binding loop can replace a region within DE loop region of CH1 or CL region. An antigen binding loop can replace a region within EF loop region of CH1 or CL region. An antigen binding loop can replace a region within FG loop region of CH1 or CL region.

In some embodiments, the binding molecule provided herein comprises one antigen binding loop. In other embodiments, the binding molecule provided herein comprises two or more antigen binding loops. For example, the binding molecule can comprise a CH1 region variant comprising two or more antigen binding loops that are introduced to and/or replace an amino acid fragment within the AB, BC, CD, DE, EF, and/or FG loop regions of the CH1 region. In other embodiments, the binding molecule comprises a CL region variant comprising two or more antigen binding loops are introduced to and/or replace an amino acid fragment within the AB, BC, CD, DE, EF, and/or FG loop regions of the CL region. In yet other embodiments, the binding molecule provided herein comprises a CH1 region variant comprising one or more antigen binding loop(s) that are introduced to and/or replace an amino acid fragment within the AB, BC, CD, DE, EF, and/or FG loop regions of the CH1 region, and a CL region variant comprising one or more antigen binding loop(s) that are introduced to and/or replace an amino acid fragment within the AB, BC, CD, DE, EF, and/or FG loop regions of the CL region. In some embodiments, the antigen binding loops are located outside of AB, BC, CD, DE, EF, and/or FG loop regions of the CH1 region. In some embodiments, the antigen binding loops are located outside of AB, BC, CD, DE, EF, and/or FG loop regions of the CL region. In some embodiments, the antigen binding loops are located in A, B, C, D, E, and/or F β-strands of the CL region. In some embodiments, the antigen binding loops are located in A, B, C, D, E, and/or F β-strands of the CH1 region.

In some embodiments, the CH1 region is a human IgG1 CH1 region comprising an amino acid sequence of

(SEQ ID NO: 1)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSC.

In some embodiments, the CH1 region comprises an amino acid sequence having at least 70%, 75%, 80%, 85%, 90% or 95% identity to SEQ ID NO:1.

In some embodiments, the CL region is a human CL kappa region comprising an amino acid sequence of

(SEQ ID NO: 2)
RTVAAPSVFIFPPSDEQLKSGTASVVCLLNNFYPREAKVQWKVDNALQS
GNSQESVTEDSKDSTYSLSSTLTLSKADYEKHKVYACEVTHQGLSSPVT
KSFNRGEC

In some embodiments, the CL region comprises an amino acid sequence having at least 70%, 75%, 80%, 85%, 90% or 95% identity to SEQ ID NO:2.

In some embodiments, the CL region is a human CL lambda region comprising an amino acid sequence of

(SEQ ID NO: 3)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS.

In some embodiments, the CL region comprises an amino acid sequence having at least 70%, 75%, 80%, 85%, 90% or 95% identity to SEQ ID NO:3.

In some embodiments, the binding molecule provided herein comprises one or two antigen binding loops in the CH1 region. In some embodiments, the binding molecule provided herein comprises one or two antigen binding loops in the CL region. In some embodiments, the binding molecule provided herein comprises one antigen binding loop at the CD loop region of the CH1 region. In some embodiments, the binding molecule provided herein comprises one antigen binding loop at the DE loop region of the CH1 region. In some embodiments, the binding molecule provided herein comprises one antigen binding loop at the CD loop region of the CH1 region and one antigen binding loop at the DE loop region of the CH1 region. In some embodiments, the binding molecule provided herein comprises one antigen binding loop at the CD loop region of the CL region. In some embodiments, the binding molecule provided herein comprises one antigen binding loop at the DE loop region of the CL region. In some embodiments, the binding molecule provided herein comprises one antigen binding loop at the CD loop region of the CL region and one antigen binding loop at the DE loop region of the CL region. In some embodiments, the binding molecule provided herein comprises one antigen binding loop at the CD loop region of the CH1 region and one antigen binding loop at the CD loop region of the CL region. In some embodiments, the binding molecule provided herein comprises one antigen binding loop at the CD loop region of the CH1 region and one antigen binding loop at the DE loop region of the CL region. In some embodiments, the binding molecule provided herein comprises one antigen binding loop at the CD loop region of the CH1 region, one antigen binding loop at the CD loop region of the CL region, and one antigen binding loop at the DE loop region of the CL region. In some embodiments, the binding molecule provided herein comprises one antigen binding loop at the DE loop region of the CH1 region and one antigen binding loop at the CD loop region of the CL region. In some embodiments, the binding molecule provided herein comprises one antigen binding loop at the DE loop region of the CH1 region and one antigen binding loop at the DE loop region of the CL region. In some embodiments, the binding molecule provided herein comprises one antigen binding loop at the DE loop region of the CH1 region, one antigen binding loop at the CD loop region of the CL region, and one antigen binding loop at the DE loop region of the CL region. In some embodiments, the binding molecule provided herein comprises one antigen binding loop at the CD loop region of the CH1 region, one antigen binding loop at the DE loop region of the CH1 region, and one antigen binding loop at the CD loop region of the CL region. In some embodiments, the binding molecule provided herein comprises one antigen binding loop at the CD loop region of the CH1 region, one antigen binding loop at the DE loop region of the CH1 region, and one antigen binding loop at the DE loop region of the CL region. In some embodiments, the binding molecule provided herein comprises one antigen binding loop at the CD loop region of the CH1 region, one antigen binding loop at the DE loop region of the CH1 region, one antigen binding loop at the CD loop region of the CL region, and one antigen binding loop at the DE loop region of the CL region.

In some specific embodiment, the antigen binding loop at the CD loop region of the CH1 region replaces the amino acid residues TSG of the CD loop of the human IgG1 CH1 region. In some specific embodiment, wherein the antigen binding loop at the DE loop region of the CH1 region replaces the amino acid residues QSS of the DE loop of the human IgG1 CH1 region. In some specific embodiment, the antigen binding loop at the CD loop region of the CL region replaces the amino acid residues SGNS of the CD loop of the human CL kappa region. In some embodiment, the antigen binding loop at the DE loop region of the CL region replaces the amino acid residues SKD of the DE loop of the human CL kappa region.

In some embodiments, the binding molecule comprises a region derived from a CH1 region of an antibody heavy chain and/or a region derived from a CL region of an antibody light chain, wherein the region derived from the CH1 region and/or the region derived from the CL region comprises one or more antigen binding loop(s).

The binding molecule provided herein can be an antibody (including any antigen binding fragments thereof). In some embodiments, the binding molecule provided herein is a multispecific or multivalent binding molecules that comprises an antigen binding domain formed by one or more constant region variant(s) provided herein.

In some embodiments, the binding molecule provided herein is in a conventional antibody format except that it comprises one or more constant region variants with one or more antigen binding loop(s), thereby introducing additional antigen binding domain(s) into the antibody to generate multispecific/multivalent antibodies. Such an exemplary binding molecule is shown in FIG. 1A. In some embodiments, the multispecific/multivalent binding molecules provided herein comprise a binding domain comprising the VH region and the VL region capable of binding to a first antigen. In addition, the multispecific binding molecules provided herein comprises an additional binding domain comprising the region derived from the CH1 region and/or the region derived from the CL region capable of binding to a second antigen.

In some embodiments, the binding molecule provided herein is a multispecific antibody. In other embodiments, the binding molecule provided herein is a multivalent antibody. The antibodies provided herein include, but are not limited to, synthetic antibodies, monoclonal antibodies, recombinantly produced antibodies, human antibodies, humanized antibodies, chimeric antibodies, etc.

In particular, the antibodies provided herein include immunoglobulin molecules and immunologically active portions of immunoglobulin molecules, i.e., molecules that contain an antigen binding site that immunospecifically binds to an antigen. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein is an IgG antibody, such as an IgG1 antibody, IgG2 antibody or IgG4 antibody (e.g., IgG4 nullbody and variants of IgG4 antibodies). In a specific embodiment, the IgG antibody is an IgG1 antibody.

In some embodiments of the various binding molecules provided herein comprises a variant and/or derivative of antibodies include antibody fragments that retain the ability to specifically bind to an epitope. In other embodiments of the various binding molecules provided herein, the first binding domain and/or the second binding domain is a variant and/or derivative of antibodies include antibody fragments that retain the ability to specifically bind to an epitope. Exemplary fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope). Derivatives of antibodies also include one or more CDR sequences of an antibody's antigen binding site. The CDR sequences may be linked together on a scaffold when two or more CDR sequences are present.

In some embodiments, the antibody provided herein is a bispecific antibody. In some embodiments, the antibody is a trispecific antibody. In some embodiments, the antibody is a quadraspecific antibody. In some embodiments, the antibody provided herein is a bivalent antibody. In some embodiments, the antibody is a trivalent antibody. In some embodiments, the antibody is a quadravalent antibody.

In one embodiment, the antibody comprises: (a) a first binding domain that binds to a first antigen, and (b) a second binding domain that binds to a second antigen. In one embodiment, the multispecific antibody comprises: (a) a first binding domain that binds to a first antigen, and (b) a second binding domain that binds to a second antigen, and (c) a third binding domain that binds to a third antigen. In one embodiment, the multispecific antibody comprises: (a) a first binding domain that binds to a first antigen, and (b) a second binding domain that binds to a second antigen, (c) a third binding domain that binds to a third antigen, and (d) a fourth binding domain that binds to a fourth antigen. In some embodiments, two or more of the first antigen, second antigen, third antigen and/or fourth antigen are the same. In some embodiments, two or more of the first antigen, second antigen, third antigen and/or fourth antigen are different.

In another aspect, provided herein is an antibody comprising: (a) a first binding domain comprising the VH region and the VL region capable of binding to a first antigen, and (b) a second binding domain comprising the region derived from the CH1 region and/or the region derived from the CL region capable of binding to a second antigen. In some embodiments, the first antigen and the second antigen are the same antigen. In some embodiments, the first antigen and the second antigen are two different antigens.

In some embodiments, the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.

In some embodiments, the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.

In some embodiments, the region derived from the CL region is a region derived from a human CL lambda region comprising an amino acid sequence of SEQ ID NO:3, and wherein the region derived from the CL region comprises an amino acid sequence having at least 70%, 75%, 80%, 85%, 90% or 95% identity to SEQ ID NO:3.

In some specific embodiments, provided herein are bispecific antibodies generated in Section 7 below.

The antibodies provided herein may be from any animal origin including birds and mammals (e.g., human, monkey, murine, donkey, sheep, rabbit, goat, guinea pig, camel, horse, or chicken). In certain embodiments, the antibodies provided herein are human or humanized monoclonal antibodies. As used herein, “human” antibodies include antibodies having the amino acid sequence of a human immunoglobulin and include antibodies isolated from human immunoglobulin libraries or from mice that express antibodies from human genes.

In certain embodiments, the antibodies are full mouse antibodies. In certain embodiments, the antibodies are mouse-human chimeric antibodies. In certain embodiments, the antibodies are humanized antibodies. In certain embodiments, the antibodies are fully human antibodies. In other embodiments, the antibodies provided herein are humanized antibodies (e.g., comprising human constant and framework regions). The antibodies provided herein may be bispecific, trispecific or of greater multispecificity.

In some embodiments, the antibody or antigen binding fragment provided herein binds the antigen with a K_{D} of less than 1000 nM. In some embodiments, the antibody or antigen binding fragment provided herein binds the antigen with a K_{D} of less than 100 nM. In some embodiments, the antibody or antigen binding fragment provided herein binds the antigen with a K_{D} of less than 50 nM. In some embodiments, the antibody or antigen binding fragment provided herein binds the antigen with a K_{D} of less than 40 nM. In some embodiments, the antibody or antigen binding fragment provided herein binds the antigen with a K_{D} of less than 30 nM. In some embodiments, the antibody or antigen binding fragment provided herein binds the antigen with a K_{D} of less than 20 nM. In some embodiments, the antibody or antigen binding fragment provided herein binds the antigen with a K_{D} of less than 10 nM. In some embodiments, the antibody or antigen binding fragment provided herein binds the antigen with a K_{D} of less than 9 nM. In some embodiments, the antibody or antigen binding fragment provided herein binds the antigen with a K_{D} of less than 8 nM. In some embodiments, the antibody or antigen binding fragment provided herein binds the antigen with a K_{D} of less than 7 nM. In some embodiments, the antibody or antigen binding fragment provided herein binds the antigen with a K_{D} of less than 6 nM. In some embodiments, the antibody or antigen binding fragment provided herein binds the antigen with a K_{D} of less than 5 nM. In some embodiments, the antibody or antigen binding fragment provided herein binds the antigen with a K_{D} of less than 4 nM. In some embodiments, the antibody or antigen binding fragment provided herein binds the antigen with a K_{D} of less than 3 nM. In some embodiments, the antibody or antigen binding fragment provided herein binds the antigen with a K_{D} of less than 2 nM. In some embodiments, the antibody or antigen binding fragment provided herein binds the antigen with a K_{D} of less than 1 nM. In some embodiments, the antibody or antigen binding fragment provided herein binds the antigen with a K_{D} of less than 0.1 nM. In some embodiments, the antibody or antigen binding fragment provided herein binds the antigen with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet® Red96 system, or by Biacore®, using, for example, a Biacore® TM-2000 or a Biacore® TM-3000. An “on-rate” or “rate of association” or “association rate” or “kon” may also be determined with the same biolayer interferometry (BLI) or surface plasmon resonance (SPR) techniques described above using, for example, the Octet® Red96, the Biacore® TM-2000, or the Biacore® TM-3000 system. In a specific embodiment, the K_{D} is determined by a Biacore® assay. In some embodiments, the antigen is a human antigen. In some embodiments, the antigen is a cynomolgus macaque antigen. In some embodiments, the antigen is a rat antigen. In other embodiments, the antigen is mouse antigen.

In some embodiments, provided herein are antibodies that specifically bind to the antigen and can modulate the antigen activity and/or expression (e.g., inhibit the antigen mediated signaling). In certain embodiments, an antigen antagonist is provided herein that is an antibody described herein that specifically binds to the antigen and inhibits (including partially inhibits) the antigen activity. In some embodiments, the antibodies provided herein inhibit (including partially inhibit or reduce) the binding of the antigen to its ligand. The antigen activity can relate to any activity of the antigen such as those known or described in the art. In certain embodiments, the antigen activity and the antigen signaling (or the antigen mediated signaling) are used interchangeably herein.

In certain embodiments, the antibody described herein attenuates (e.g., partially attenuates) the antigen activity. In some embodiments, the antibody provided herein attenuates the antigen activity by at least about 10%. In some embodiments, the antibody provided herein attenuates the antigen activity by at least about 20%. In some embodiments, the antibody provided herein attenuates the antigen activity by at least about 30%. In some embodiments, the antibody provided herein attenuates the antigen activity by at least about 40%. In some embodiments, the antibody provided herein attenuates the antigen activity by at least about 50%. In some embodiments, the antibody provided herein attenuates the antigen activity by at least about 60%. In some embodiments, the antibody provided herein attenuates the antigen activity by at least about 70%. In some embodiments, the antibody provided herein attenuates the antigen activity by at least about 80%. In some embodiments, the antibody provided herein attenuates the antigen activity by at least about 90%. In some embodiments, the antibody provided herein attenuates the antigen activity by at least about 95%. In certain embodiments, the antibody described herein can attenuate (e.g., partially attenuate) the antigen activity by at least about 15% to about 65%. In certain embodiments, the antibody described herein can attenuate (e.g., partially attenuate) the antigen activity by at least about 20% to about 65%. In certain embodiments, the antibody described herein can attenuate (e.g., partially attenuate) the antigen activity by at least about 30% to about 65%.

In specific embodiments, the attenuation of the antigen activity is assessed by methods described herein. In specific embodiments, the attenuation of the antigen activity is assessed by methods known to one of skill in the art. In certain embodiments, the attenuation of the antigen activity is relative to the antigen activity in the presence of stimulation without any antibody against the antigen. In certain embodiments, the attenuation of the antigen activity is relative to the antigen activity in the presence of stimulation with an unrelated antibody (e.g., an antibody that does not specifically bind to the antigen).

A non-limiting example of the antigen activity is the antigen mediated signaling. Thus, in certain embodiments, the antibody described herein attenuates (e.g., partially attenuates) the antigen mediated signaling. In some embodiments, the antibody provided herein attenuates the antigen mediated signaling by at least about 10%. In some embodiments, the antibody provided herein attenuates the antigen mediated signaling by at least about 20%. In some embodiments, the antibody provided herein attenuates the antigen mediated signaling by at least about 30%. In some embodiments, the antibody provided herein attenuates the antigen mediated signaling by at least about 40%. In some embodiments, the antibody provided herein attenuates the antigen mediated signaling by at least about 50%. In some embodiments, the antibody provided herein attenuates the antigen mediated signaling by at least about 60%. In some embodiments, the antibody provided herein attenuates the antigen mediated signaling by at least about 70%. In some embodiments, the antibody provided herein attenuates the antigen mediated signaling by at least about 80%. In some embodiments, the antibody provided herein attenuates the antigen mediated signaling by at least about 90%. In some embodiments, the antibody provided herein attenuates the antigen mediated signaling by at least about 95%. In certain embodiments, the antibody described herein can attenuate (e.g., partially attenuate) the antigen mediated signaling by at least about 15% to about 65%. In certain embodiments, the antibody described herein can attenuate (e.g., partially attenuate) the antigen mediated signaling by at least about 20% to about 65%. In certain embodiments, the antibody described herein can attenuate (e.g., partially attenuate) the antigen mediated signaling by at least about 30% to about 65%.

In some embodiments, one antigen bound by the present binding molecules is an antigen on the surface of a target cell such as a cancer cell. In some embodiments, the antigen is a tumor-specific antigen, a tumor-associated antigen, or a neoantigen.

In some embodiments, the target cell is a cancer cell, e.g., a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer. In some embodiments, the cancer is an anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor, is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In some embodiments, one antigen bound by the present binding molecule is a cancer antigen. In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R. In some embodiments, the cancer antigen is angiopoietin. In some embodiments, the cancer antigen is BCMA. In some embodiments, the cancer antigen is CD19. In some embodiments, the cancer antigen is CD20. In some embodiments, the cancer antigen is CD22. In some embodiments, the cancer antigen is CD25 (IL2-R). In some embodiments, the cancer antigen is CD30. In some embodiments, the cancer antigen is CD33. In some embodiments, the cancer antigen is CD37. In some embodiments, the cancer antigen is CD38. In some embodiments, the cancer antigen is CD52. In some embodiments, the cancer antigen is CD56. In some embodiments, the cancer antigen is CD123 (IL-3R). In some embodiments, the cancer antigen is cMET. In some embodiments, the cancer antigen is DLL/Notch. In some embodiments, the cancer antigen is EGFR. In some embodiments, the cancer antigen is EpCAM. In some embodiments, the cancer antigen is FGF. In some embodiments, the cancer antigen is FGF-R. In some embodiments, the cancer antigen is GD2. In some embodiments, the cancer antigen is HER2. In some embodiments, the cancer antigen is Mesothelin. In some embodiments, the cancer antigen is Nectin-4. In some embodiments, the cancer antigen is PDGFRα. In some embodiments, the cancer antigen is RANKL. In some embodiments, the cancer antigen is SLAMF7. In some embodiments, the cancer antigen is TROP2. In some embodiments, the cancer antigen is VEGF. In some embodiments, the cancer antigen is VEGF-R.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-B1. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

In some embodiments, the present binding molecule binds to a B cell antigen. In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267, CD268, CD269, CD270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen. In some embodiments, the B cell antigen is a CD1a antigen. In some embodiments, the B cell antigen is a CD1b antigen. In some embodiments, the B cell antigen is a CD1c antigen. In some embodiments, the B cell antigen is a CD1d antigen. In some embodiments, the B cell antigen is a CD2 antigen. In some embodiments, the B cell antigen is a CD5 antigen. In some embodiments, the B cell antigen is a CD6 antigen. In some embodiments, the B cell antigen is a CD9 antigen. In some embodiments, the B cell antigen is a CD11a antigen. In some embodiments, the B cell antigen is a CD11b antigen. In some embodiments, the B cell antigen is a CD11c antigen. In some embodiments, the B cell antigen is a CD17 antigen. In some embodiments, the B cell antigen is a CD18 antigen. In some embodiments, the B cell antigen is a CD19 antigen. In some embodiments, the B cell antigen is a CD20 antigen. In some embodiments, the B cell antigen is a CD21 antigen. In some embodiments, the B cell antigen is a CD22 antigen. In some embodiments, the B cell antigen is a CD23 antigen. In some embodiments, the B cell antigen is a CD24 antigen. In some embodiments, the B cell antigen is a CD25 antigen. In some embodiments, the B cell antigen is a CD26 antigen. In some embodiments, the B cell antigen is a CD27 antigen. In some embodiments, the B cell antigen is a CD29 antigen. In some embodiments, the B cell antigen is a CD30 antigen. In some embodiments, the B cell antigen is a CD31 antigen. In some embodiments, the B cell antigen is a CD32a antigen. In some embodiments, the B cell antigen is a CD32b antigen. In some embodiments, the B cell antigen is a CD35 antigen. In some embodiments, the B cell antigen is a CD37 antigen. In some embodiments, the B cell antigen is a CD38 antigen. In some embodiments, the B cell antigen is a CD39 antigen. In some embodiments, the B cell antigen is a CD40 antigen. In some embodiments, the B cell antigen is a CD45 antigen. In some embodiments, the B cell antigen is a CD45RA antigen. In some embodiments, the B cell antigen is a CD45RB antigen. In some embodiments, the B cell antigen is a CD45RC antigen. In some embodiments, the B cell antigen is a CD45RO antigen. In some embodiments, the B cell antigen is a CD46 antigen. In some embodiments, the B cell antigen is a CD47 antigen. In some embodiments, the B cell antigen is a CD48 antigen. In some embodiments, the B cell antigen is a CD49b antigen. In some embodiments, the B cell antigen is a CD49c antigen. In some embodiments, the B cell antigen is a CD49d antigen. In some embodiments, the B cell antigen is a CD50 antigen. In some embodiments, the B cell antigen is a CD52 antigen. In some embodiments, the B cell antigen is a CD53 antigen. In some embodiments, the B cell antigen is a CD54 antigen. In some embodiments, the B cell antigen is a CD55 antigen. In some embodiments, the B cell antigen is a CD58 antigen. In some embodiments, the B cell antigen is a CD60a antigen. In some embodiments, the B cell antigen is a CD62L antigen. In some embodiments, the B cell antigen is a CD63 antigen. In some embodiments, the B cell antigen is a CD68 antigen. In some embodiments, the B cell antigen is a CD69 antigen. In some embodiments, the B cell antigen is a CD70 antigen. In some embodiments, the B cell antigen is a CD72 antigen. In some embodiments, the B cell antigen is a CD73 antigen. In some embodiments, the B cell antigen is a CD74 antigen. In some embodiments, the B cell antigen is a CD75 antigen. In some embodiments, the B cell antigen is a CD75S antigen. In some embodiments, the B cell antigen is a CD77 antigen. In some embodiments, the B cell antigen is a CD79a antigen. In some embodiments, the B cell antigen is a CD79b antigen. In some embodiments, the B cell antigen is a CD80 antigen. In some embodiments, the B cell antigen is a CD81 antigen. In some embodiments, the B cell antigen is a CD82 antigen. In some embodiments, the B cell antigen is a CD83 antigen. In some embodiments, the B cell antigen is a CD84 antigen. In some embodiments, the B cell antigen is a CD85E antigen. In some embodiments, the B cell antigen is a CD85I antigen. In some embodiments, the B cell antigen is a CD85J antigen. In some embodiments, the B cell antigen is a CD86 antigen. In some embodiments, the B cell antigen is a CD92 antigen. In some embodiments, the B cell antigen is a CD95 antigen. In some embodiments, the B cell antigen is a CD97 antigen. In some embodiments, the B cell antigen is a CD98 antigen. In some embodiments, the B cell antigen is a CD99 antigen. In some embodiments, the B cell antigen is a CD100 antigen. In some embodiments, the B cell antigen is a CD102 antigen. In some embodiments, the B cell antigen is a CD108 antigen. In some embodiments, the B cell antigen is a CD119 antigen. In some embodiments, the B cell antigen is a CD120a antigen. In some embodiments, the B cell antigen is a CD120b antigen. In some embodiments, the B cell antigen is a CD121b antigen. In some embodiments, the B cell antigen is a CD122 antigen. In some embodiments, the B cell antigen is a CD124 antigen. In some embodiments, the B cell antigen is a CD125 antigen. In some embodiments, the B cell antigen is a CD126 antigen. In some embodiments, the B cell antigen is a CD130 antigen. In some embodiments, the B cell antigen is a CD132 antigen. In some embodiments, the B cell antigen is a CD137 antigen. In some embodiments, the B cell antigen is a CD138 antigen. In some embodiments, the B cell antigen is a CD139 antigen. In some embodiments, the B cell antigen is a CD147 antigen. In some embodiments, the B cell antigen is a CD148 antigen. In some embodiments, the B cell antigen is a CD150 antigen. In some embodiments, the B cell antigen is a CD152 antigen. In some embodiments, the B cell antigen is a CD162 antigen. In some embodiments, the B cell antigen is a CD164 antigen. In some embodiments, the B cell antigen is a CD166 antigen. In some embodiments, the B cell antigen is a CD167a antigen. In some embodiments, the B cell antigen is a CD170 antigen. In some embodiments, the B cell antigen is a CD171 antigen. In some embodiments, the B cell antigen is a CD175 antigen. In some embodiments, the B cell antigen is a CD175s antigen. In some embodiments, the B cell antigen is a CD180 antigen. In some embodiments, the B cell antigen is a CD184 antigen. In some embodiments, the B cell antigen is a CD185 antigen. In some embodiments, the B cell antigen is a CD192 antigen. In some embodiments, the B cell antigen is a CD196 antigen. In some embodiments, the B cell antigen is a CD197 antigen. In some embodiments, the B cell antigen is a CD200 antigen. In some embodiments, the B cell antigen is a CD205 antigen. In some embodiments, the B cell antigen is a CD201a antigen. In some embodiments, the B cell antigen is a CDw210b antigen. In some embodiments, the B cell antigen is a CD212 antigen. In some embodiments, the B cell antigen is a CD213a1 antigen. In some embodiments, the B cell antigen is a CD213a2 antigen. In some embodiments, the B cell antigen is a CD215 antigen. In some embodiments, the B cell antigen is a CD217 antigen. In some embodiments, the B cell antigen is a CD218a antigen. In some embodiments, the B cell antigen is a CD218b antigen. In some embodiments, the B cell antigen is a CD220 antigen. In some embodiments, the B cell antigen is a CD221 antigen. In some embodiments, the B cell antigen is a CD222 antigen. In some embodiments, the B cell antigen is a CD224 antigen. In some embodiments, the B cell antigen is a CD225 antigen. In some embodiments, the B cell antigen is a CD226 antigen. In some embodiments, the B cell antigen is a CD227 antigen. In some embodiments, the B cell antigen is a CD229 antigen. In some embodiments, the B cell antigen is a CD230 antigen. In some embodiments, the B cell antigen is a CD232 antigen. In some embodiments, the B cell antigen is a CD252 antigen. In some embodiments, the B cell antigen is a CD252 antigen. In some embodiments, the B cell antigen is a CD254 antigen. In some embodiments, the B cell antigen is a CD255 antigen. In some embodiments, the B cell antigen is a CD256 antigen. In some embodiments, the B cell antigen is a CD257 CD258 antigen. In some embodiments, the B cell antigen is a CD259 antigen. In some embodiments, the B cell antigen is a CD260 antigen. In some embodiments, the B cell antigen is a CD261 antigen. In some embodiments, the B cell antigen is a CD262 antigen. In some embodiments, the B cell antigen is a CD263 antigen. In some embodiments, the B cell antigen is a CD264 antigen. In some embodiments, the B cell antigen is a CD267 antigen. In some embodiments, the B cell antigen is a CD268 antigen. In some embodiments, the B cell antigen is a CD269 antigen. In some embodiments, the B cell antigen is a CD270 antigen. In some embodiments, the B cell antigen is a CD272 antigen. In some embodiments, the B cell antigen is a CD274 antigen. In some embodiments, the B cell antigen is a CD275 antigen. In some embodiments, the B cell antigen is a CD277 antigen. In some embodiments, the B cell antigen is a CD279 antigen. In some embodiments, the B cell antigen is a CD283 antigen. In some embodiments, the B cell antigen is a CD289 antigen. In some embodiments, the B cell antigen is a CD290 antigen. In some embodiments, the B cell antigen is a CD295 antigen. In some embodiments, the B cell antigen is a CD298 antigen. In some embodiments, the B cell antigen is a CD300 antigen. In some embodiments, the B cell antigen is a CD300c antigen. In some embodiments, the B cell antigen is a CD305 antigen. In some embodiments, the B cell antigen is a CD306 antigen. In some embodiments, the B cell antigen is a CD307a antigen. In some embodiments, the B cell antigen is a CD307b antigen. In some embodiments, the B cell antigen is a CD307c antigen. In some embodiments, the B cell antigen is a CD307d antigen. In some embodiments, the B cell antigen is a CD307e antigen. In some embodiments, the B cell antigen is a CD314 antigen. In some embodiments, the B cell antigen is a CD215 antigen. In some embodiments, the B cell antigen is a CD316 antigen. In some embodiments, the B cell antigen is a CD317 antigen. In some embodiments, the B cell antigen is a CD319 antigen. In some embodiments, the B cell antigen is a CD321 antigen. In some embodiments, the B cell antigen is a CD327 antigen. In some embodiments, the B cell antigen is a CD328 antigen. In some embodiments, the B cell antigen is a CD329 antigen. In some embodiments, the B cell antigen is a CD338 antigen. In some embodiments, the B cell antigen is a CD351 antigen. In some embodiments, the B cell antigen is a CD352 antigen. In some embodiments, the B cell antigen is a CD353 antigen. In some embodiments, the B cell antigen is a CD354 antigen. In some embodiments, the B cell antigen is a CD355 antigen. In some embodiments, the B cell antigen is a CD356 antigen. In some embodiments, the B cell antigen is a CD357 antigen. In some embodiments, the B cell antigen is a CD358 antigen. In some embodiments, the B cell antigen is a CD360 antigen. In some embodiments, the B cell antigen is a CD361 antigen. In some embodiments, the B cell antigen is a CD362 antigen. In some embodiments, the B cell antigen is a CD363 antigen.

In one embodiment, the present binding molecule binds a pathogen. In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM). In some embodiments, the infectious disease is Anaplasmosis. In some embodiments, the infectious disease is Anthrax. In some embodiments, the infectious disease is Babesiosis. In some embodiments, the infectious disease is Botulism. In some embodiments, the infectious disease is Brucellosis. In some embodiments, the infectious disease is Campylobacteriosis. In some embodiments, the infectious disease is Carbapenem-resistant Infection. In some embodiments, the infectious disease is Chancroid. In some embodiments, the infectious disease is Chikungunya Virus Infection. In some embodiments, the infectious disease is Chlamydia. In some embodiments, the infectious disease is Ciguatera. In some embodiments, the infectious disease is Difficile Infection. In some embodiments, the infectious disease is Perfringens. In some embodiments, the infectious disease is Coccidioidomycosis fungal infection. In some embodiments, the infectious disease is coronavirus. In some embodiments, the infectious disease is Covid-19 (SARS-COV-2). In some embodiments, the infectious disease is Creutzfeldt-Jacob Disease/transmissible spongiform encephalopathy. In some embodiments, the infectious disease is Cryptosporidiosis (Crypto). In some embodiments, the infectious disease is Cyclosporiasis. In some embodiments, the infectious disease is Dengue 1, 2, 3 or 4. In some embodiments, the infectious disease is Diphtheria. In some embodiments, the infectious disease is E. coli infection/Shiga toxin-producing (STEC). In some embodiments, the infectious disease is Eastern Equine Encephalitis. In some embodiments, the infectious disease is Hemorrhagic Fever (Ebola). In some embodiments, the infectious disease is Ehrlichiosis. In some embodiments, the infectious disease is Encephalitis. In some embodiments, the infectious disease is Arboviral or parainfectious. In some embodiments, the infectious disease is Non-Polio Enterovirus. In some embodiments, the infectious disease is D68 Enteroviru(EV-D68). In some embodiments, the infectious disease is Giardiasis. In some embodiments, the infectious disease is Glanders. In some embodiments, the infectious disease is Gonococcal Infection. In some embodiments, the infectious disease is Granuloma inguinale. In some embodiments, the infectious disease is Haemophilus Influenza disease Type B (Hib or H-flu). In some embodiments, the infectious disease is Hantavirus Pulmonary Syndrome (HPS). In some embodiments, the infectious disease is Hemolytic Uremic Syndrome (HUS). In some embodiments, the infectious disease is Hepatitis A (Hep A). In some embodiments, the infectious disease is Hepatitis B (Hep B). In some embodiments, the infectious disease is Hepatitis C (Hep C). In some embodiments, the infectious disease is Hepatitis D (Hep D). In some embodiments, the infectious disease is Hepatitis E (Hep E). In some embodiments, the infectious disease is Herpes. In some embodiments, the infectious disease is Herpes Zoster (Shingles). In some embodiments, the infectious disease is Histoplasmosis infection. In some embodiments, the infectious disease is Human Immunodeficiency Virus/AIDS (HIV/AIDS). In some embodiments, the infectious disease is Human Papillomavirus (HPV). In some embodiments, the infectious disease is Influenza (Flu). In some embodiments, the infectious disease is Legionellosis (Legionnaires Disease). In some embodiments, the infectious disease is Leprosy (Hansens Disease). In some embodiments, the infectious disease is Leptospirosis. In some embodiments, the infectious disease is Listeriosis (Listeria). In some embodiments, the infectious disease is Lyme Disease. In some embodiments, the infectious disease is Lymphogranuloma venereum infection (LGV). In some embodiments, the infectious disease is Malaria. In some embodiments, the infectious disease is Measles. In some embodiments, the infectious disease is Melioidosis. In some embodiments, the infectious disease is Meningitis (Viral). In some embodiments, the infectious disease is Meningococcal Disease (Meningitis (Bacterial)). In some embodiments, the infectious disease is Middle East Respiratory Syndrome Coronavirus (MERS-COV). In some embodiments, the infectious disease is Mumps. In some embodiments, the infectious disease is Norovirus. In some embodiments, the infectious disease is Pediculosis. In some embodiments, the infectious disease is Pelvic Inflammatory Disease (PID). In some embodiments, the infectious disease is Pertussis (Whooping Cough). In some embodiments, the infectious disease is Plague (Bubonic. In some embodiments, the infectious disease is Septicemic. In some embodiments, the infectious disease is Pneumonic). In some embodiments, the infectious disease is Pneumococcal Disease (Pneumonia). In some embodiments, the infectious disease is Poliomyelitis (Polio). In some embodiments, the infectious disease is Powassan. In some embodiments, the infectious disease is Psittacosis. In some embodiments, the infectious disease is Pthiriasis. In some embodiments, the infectious disease is Pustular Rash diseases (Small pox. In some embodiments, the infectious disease is monkeypox. In some embodiments, the infectious disease is cowpox). In some embodiments, the infectious disease is Q-Fever. In some embodiments, the infectious disease is Rabies. In some embodiments, the infectious disease is Rickettsiosis (Rocky Mountain Spotted Fever). In some embodiments, the infectious disease is Rubella (German Measles). In some embodiments, the infectious disease is Salmonellosis gastroenteritis (Salmonella). In some embodiments, the infectious disease is Scabies. In some embodiments, the infectious disease is Scombroid. In some embodiments, the infectious disease is Sepsis. In some embodiments, the infectious disease is Severe Acute Respiratory Syndrome (SARS). In some embodiments, the infectious disease is Shigellosis gastroenteritis (Shigella). In some embodiments, the infectious disease is Smallpox. In some embodiments, the infectious disease is Staphyloccal Infection Methicillin-resistant (MRSA). In some embodiments, the infectious disease is Staphylococcal Food Poisoning Enterotoxin B Poisoning (Staph Food Poisoning). In some embodiments, the infectious disease is Saphylococcal Infection Vancomycin Intermediate (VISA). In some embodiments, the infectious disease is Staphylococcal Infection Vancomycin Resistant (VRSA). In some embodiments, the infectious disease is Streptococcal Disease Group A (invasive) (Strep A (invasive). In some embodiments, the infectious disease is Streptococcal Disease. In some embodiments, the infectious disease is Group B (Strep-B). In some embodiments, the infectious disease is Streptococcal Toxic-Shock Syndrome STSS Toxic Shock. In some embodiments, the infectious disease is Syphilis (primary. In some embodiments, the infectious disease is secondary. In some embodiments, the infectious disease is early latent. In some embodiments, the infectious disease is late latent. In some embodiments, the infectious disease is congenital). In some embodiments, the infectious disease is Tetanus Infection. In some embodiments, the infectious disease is Trichomoniasis. In some embodiments, the infectious disease is Trichonosis Infection. In some embodiments, the infectious disease is Tuberculosis (TB). In some embodiments, the infectious disease is Tuberculosis Latent (LTBI). In some embodiments, the infectious disease is Tularemia. In some embodiments, the infectious disease is Typhoid Fever Group D. In some embodiments, the infectious disease is Vaginosis. In some embodiments, the infectious disease is Varicella (Chickenpox), Vibrio cholerae (Cholera). In some embodiments, the infectious disease is Vibriosis (Vibrio). In some embodiments, the infectious disease is Ebola Virus Hemorrhagic Fever. In some embodiments, the infectious disease is Lasa Virus Hemorrhagic Fever. In some embodiments, the infectious disease is Marburg Virus Hemorrhagic Fever. In some embodiments, the infectious disease is West Nile Virus. In some embodiments, the infectious disease is Yellow Fever. In some embodiments, the infectious disease is Yersenia. In some embodiments, the infectious disease is and Zika Virus Infection.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments, the virus is a virus of the adenoviridae family. In some embodiments, the virus is a virus of the arenaviridae family. In some embodiments, the virus is a virus of the astroviridae family. In some embodiments, the virus is a virus of the bunyaviridae family. In some embodiments, the virus is a virus of the caliciviridae family. In some embodiments, the virus is a virus of the coronaviridae family. In some embodiments, the virus is a virus of the filoviridae family. In some embodiments, the virus is a virus of the flaviviridae family. In some embodiments, the virus is a virus of the hepadnaviridae family. In some embodiments, the virus is a virus of the hepeviridae family. In some embodiments, the virus is a virus of the orthomyxoviridae family. In some embodiments, the virus is a virus of the papillomaviridae family. In some embodiments, the virus is a virus of the paramyxoviridae family. In some embodiments, the virus is a virus of the parvoviridae family. In some embodiments, the virus is a virus of the picornaviridae family. In some embodiments, the virus is a virus of the polyomaviridae family. In some embodiments, the virus is a virus of the poxviridae family. In some embodiments, the virus is a virus of the reoviridae family. In some embodiments, the virus is a virus of the retroviridae family. In some embodiments, the virus is a virus of the rhabdoviridae family. In some embodiments, the virus is a virus of the togaviridae family.

In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments, the virus is an adenovirus. In some embodiments, the virus is a coronavirus. In some embodiments, the coronavirus virus is Covid-19 (SARS-COV-2). In some embodiments, the virus is a coxsackievirus. In some embodiments, the virus is a Epstein-Barr virus. In some embodiments, the virus is a hepatitis A virus. In some embodiments, the virus is a hepatitis B virus. In some embodiments, the virus is a hepatitis C virus. In some embodiments, the virus is a herpes simplex virus type 2. In some embodiments, the virus is a cytomegalovirus. In some embodiments, the virus is a human herpes virus type 8. In some embodiments, the virus is a human immunodeficiency virus. In some embodiments, the virus is an influenza virus. In some embodiments, the virus is a measles virus. In some embodiments, the virus is a mumps virus. In some embodiments, the virus is a human papillomavirus. In some embodiments, the virus is a parainfluenza virus. In some embodiments, the virus is a poliovirus. In some embodiments, the virus is a rabies virus. In some embodiments, the virus is a respiratory syncytial virus. In some embodiments, the virus is a rubella virus. In some embodiments, the virus is a varicella-zoster virus.

In some embodiments, the pathogen is a bacteria. In some embodiments, the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus. In some embodiments, the bacteria is a bacteria of the bacillus genus. In some embodiments, the bacteria is a bacteria of the bartonella genus. In some embodiments, the bacteria is a bacteria of the bordetella genus. In some embodiments, the bacteria is a bacteria of the borrelia genus. In some embodiments, the bacteria is a bacteria of the brucella genus. In some embodiments, the bacteria is a bacteria of the campylobacter genus. In some embodiments, the bacteria is a bacteria of the chlamydia genus. In some embodiments, the bacteria is a bacteria of the chlamydophila genus. In some embodiments, the bacteria is a bacteria of the clostridium genus. In some embodiments, the bacteria is a bacteria of the corynebacterium genus. In some embodiments, the bacteria is a bacteria of the enterococcus genus. In some embodiments, the bacteria is a bacteria of the escherichia genus. In some embodiments, the bacteria is a bacteria of the francisella genus. In some embodiments, the bacteria is a bacteria of the haemophilus genus. In some embodiments, the bacteria is a bacteria of the helicobacter genus. In some embodiments, the bacteria is a bacteria of the legionella genus. In some embodiments, the bacteria is a bacteria of the leptospira genus. In some embodiments, the bacteria is a bacteria of the listeria genus. In some embodiments, the bacteria is a bacteria of the mycobacterium genus. In some embodiments, the bacteria is a bacteria of the mycoplasma genus. In some embodiments, the bacteria is a bacteria of the neisseria genus. In some embodiments, the bacteria is a bacteria of the pseudomonas genus. In some embodiments, the bacteria is a bacteria of the rickettsia genus. In some embodiments, the bacteria is a bacteria of the salmonella genus. In some embodiments, the bacteria is a bacteria of the shigella genus. In some embodiments, the bacteria is a bacteria of the staphylococcus genus. In some embodiments, the bacteria is a bacteria of the streptococcus genus. In some embodiments, the bacteria is a bacteria of the treponema genus. In some embodiments, the bacteria is a bacteria of the ureaplasma genus. In some embodiments, the bacteria is a bacteria of the vibrio genus. In some embodiments, the bacteria is a bacteria of the yersinia genus.

In some embodiments, the pathogen is a parasite. In some embodiments, the parasite is a protozoa, helminth, or ectoparasite. In some embodiments, the protozoa is an entamoeba, giardia, leishmania, balantidium, plasmodium, or cryptosporidium. In some embodiments, the helminth is a trematode, cestode, acanthocephalan, or round worm. In some embodiments, the ectoparasite is a arthropod.

The present constant region variant can be introduced into any existing multispecific antibody platform or formats known in the art, including any known bispecific antibody formats in the art, to provide one or more additional antigen binding domain(s).

Such known multispecific antibody formats include a multispecific antibody obtained via a controlled Fab arm exchange. The multispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Other examples can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope. Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

In addition to methods described above, another known bispecific antibody format can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

### 5.2.1 Monoclonal Antibodies

The antibodies (including multispecific or multivalent antibodies) of the present disclosure can be or derived from monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium, which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., 1987, Monoclonal Antibody Production Techniques and Applications 51-63).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Plückthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In phage display methods, functional antibody domains are displayed on the surface of phage particles which carry the polynucleotide sequences encoding them. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display antigen binding site of antibody fused to phage coat protein. Such phage libraries are screened against the desired antigen. Clones expressing antigen binding sites capable of binding to the desired antigen are adsorbed to the antigen and thus separated from the non-binding clones in the library. The binding clones are then eluted from the antigen and can be further enriched by additional cycles of antigen adsorption/elution.

The antigen binding site can be within the variable domain of the antibody. Variable domains can be displayed functionally on phage as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

The antigen binding site can be outside of the variable domain of the antibody. For instance, the antigen binding site can be in the constant region of the antibody. Constant region libraries can be constructed by replacing structural loops of the constant region with one or more highly variable antigen binding loops. Such constant region libraries are described in more detail in Section 5.4 below.

Screening of the libraries can be accomplished by various techniques known in the art. For example, a specific antigen (e.g., polypeptide, fragment, or epitope of the antigen) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Antibodies can be obtained by designing a suitable antigen screening procedure to select for the phage clone of interest followed by construction of a full length antibody clone using VH and/or VL sequences (e.g., the Fv sequences), various CDR sequences from VH and VL sequences, or other antigen binding sequences from the phage clone of interest and suitable constant region (e.g., Fc) sequences described in Kabat et al., supra.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies or antigen binding fragments (e.g., Fab) produced using techniques such as those described herein can be isolated using standard, well known techniques. For example, antibodies or antigen binding fragments can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized.

### 5.2.2 Antibody Fragments

The present disclosure provides multispecific antibodies comprising antibody fragments that bind to more than one antigen. In some embodiments, provided herein is a binding molecule comprising one or more antibody fragments and one or more constant region variant(s) provided herein.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. scFv fusion proteins may be constructed to yield fusion of a binding molecule provided herein at either the amino or the carboxy terminus of a scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains. sdAb fusion proteins may be constructed to yield fusion of a binding molecule provided herein at either the amino or the carboxy terminus of a sdAb.

Antibodies provided herein include, but are not limited to, immunoglobulin molecules and immunologically active portions of immunoglobulin molecules. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule. In a specific embodiment, an antibody provided herein is an IgG antibody, such as an IgG1 antibody, IgG2 antibody or IgG4 antibody (e.g., IgG4 nullbody and variants of IgG4 antibodies). In a specific embodiment, the IgG antibody is an IgG1 antibody. In some embodiments, the IgG antibody comprises a Fc region with mutations to enhance Fc effector functions.

Variants and derivatives of antibodies include antibody functional fragments that retain the ability to bind to a specific antigen. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope).

### 5.2.3 Humanized Antibodies

The antibodies described herein can, for example, include humanized antibodies, e.g., deimmunized or composite human antibodies.

A humanized antibody can comprise human framework region and human constant region sequences. For example, a humanized antibody can comprise human constant region sequences. In certain embodiments, a humanized antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592, 106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp): 5973s-5977s (1995), Couto et al., Cancer Res. 55(8): 1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

### 5.2.4 Human Antibodies

In specific embodiments, the antibody provided herein comprises a fully human antibody or fragment thereof. Fully human antibodies may be produced by any method known in the art. Human antibodies provided herein can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain Fab chimeras. Selection with antigen results in isolation of a non-human chain/human chain chimeric Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 5.2.5 Fc Engineering

It may be desirable to modify an antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments of each or any of the above- or below-mentioned embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody.

In certain embodiments, the modification to the Fc region of the antibody results in the enhancement of an effector function of the antibody. In certain embodiments, the effector function is ADCC, ADCP, and/or CDC. In some embodiments of each or any of the above- or below-mentioned embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. In some embodiments of each or any of the above- or below-mentioned embodiments, Knobs-in-holes (KIH) technology was used to engineer the antibody.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 5.2.6 Alternative Binding Agents

The present disclosure encompasses non-immunoglobulin binding agents that specifically bind to the same epitope as an antibody disclosed herein. In some embodiments of each or any of the above- or below-mentioned embodiments, a non-immunoglobulin binding agent is identified as an agent that displaces or is displaced by an antibody of the present disclosure in a competitive binding assay. These alternative binding agents may include, for example, any of the engineered protein scaffolds known in the art. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac 7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### 5.2.7 Antibody Variants

In some embodiments of each or any of the above- or below-mentioned embodiments, amino acid sequence modification(s) of antibodies or antigen binding fragments are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments of each or any of the above- or below-mentioned embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the antibody variant DNA.

Any cysteine residue not involved in maintaining the proper conformation of the antibody provided herein also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In some embodiments of each or any of the above- or below-mentioned embodiments, an antibody molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, for example, by in vitro determination of T-cell epitopes or in silico prediction of such epitopes, as known in the art. Once the critical residues for T-cell epitope function have been identified, mutations can be made to remove immunogenicity and retain antibody activity. For review, see, for example, Jones et al., 2009, Methods in Molecular Biology 525:405-23.

### 5.2.8 In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the Fab. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments of each or any of the above- or below-mentioned embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, the antibodies can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 5.2.9 Antibody Modifications

Covalent modifications of the antibodies binding to a specific antigen are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody provided herein included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody of the present disclosure may also be modified to form chimeric molecules comprising the antibody fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are panels of antibodies that bind to a specific antigen. In specific embodiments, the panels of antibodies have different association rates, different dissociation rates, different affinities for a specific antigen, and/or different specificities for a specific antigen. In some embodiments of each or any of the above- or below-mentioned embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. In some embodiments of each or any of the above- or below-mentioned embodiments, the panels comprise about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, about 1000, about 2000 or about 3000 antibodies or any number of antibodies within a range defined by any two aforementioned values. Panels of antibodies can be used, for example, in 96-well or 384-well plates, for assays such as ELISAs.

### 5.2.10 Immunoconjugates

The present disclosure also provides conjugates comprising any one of the antibodies of the present disclosure covalently bound by a synthetic linker to one or more non-antibody agents.

In some embodiments of each or any of the above- or below-mentioned embodiments, antibodies provided herein are conjugated or recombinantly fused, e.g., to a therapeutic agent (e.g., a cytotoxic agent) or a diagnostic or detectable molecule. The conjugated or recombinantly fused antibodies can be useful, for example, for treating or preventing a disease or disorder. The conjugated or recombinantly fused antibodies can be useful, for example, for monitoring or prognosing the onset, development, progression, and/or severity of a disease or disorder.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (131I, 125I, 123I, and 121I,), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., CDR1, CDR2, and/or CDR3) and a heterologous protein, polypeptide, or peptide. In one embodiment, the heterologous protein, polypeptide, or peptide that the antibody is fused to is useful for targeting the antibody to a particular cell type.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the &-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50): 12047-57).

### 5.3 Polynucleotides

In certain embodiments, the disclosure encompasses polynucleotides that encode the antibodies described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag supplied by a vector that allows efficient purification of the polypeptide fused to the marker in the case of a bacterial host. In some embodiments of each or any of the above- or below-mentioned embodiments, a marker is used in conjunction with other affinity tags.

The present disclosure further relates to variants of the polynucleotides described herein, wherein the variant encodes, for example, fragments, analogs, and/or derivatives of a polypeptide. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an antibody or antigen binding fragment thereof described herein.

As used herein, the phrase “a polynucleotide having a nucleotide sequence at least, for example, 95% ‘identical’ to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments of each or any of the above- or below-mentioned embodiments, a polynucleotide variant contains alterations that produce silent substitutions, additions, or deletions, but does not alter the properties or activities of the encoded polypeptide. In some embodiments of each or any of the above- or below-mentioned embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments of each or any of the above- or below-mentioned embodiments, a polynucleotide variant comprises at least one silent mutation in a non-coding or a coding region of the sequence.

In some embodiments of each or any of the above- or below-mentioned embodiments, a polynucleotide variant is produced to modulate or alter expression (or expression levels) of the encoded polypeptide. In some embodiments of each or any of the above- or below-mentioned embodiments, a polynucleotide variant is produced to increase expression of the encoded polypeptide. In some embodiments of each or any of the above- or below-mentioned embodiments, a polynucleotide variant is produced to decrease expression of the encoded polypeptide. In some embodiments of each or any of the above- or below-mentioned embodiments, a polynucleotide variant has increased expression of the encoded polypeptide as compared to a parental polynucleotide sequence. In some embodiments of each or any of the above- or below-mentioned embodiments, a polynucleotide variant has decreased expression of the encoded polypeptide as compared to a parental polynucleotide sequence.

In certain embodiments, the present disclosure provides a polynucleotide comprising a nucleotide sequence at least about 80% identical, at least about 85% identical, at least about 90% identical, at least about 95% identical, and in some embodiments, at least about 96%, 97%, 98% or 99% identical to a polynucleotide listed in the Sequence Listing provided herein.

In certain embodiments, the present disclosure provides a polynucleotide comprising a nucleotide sequence at least about 80% identical, at least about 85% identical, at least about 90% identical, at least about 95% identical, and in some embodiments, at least about 96%, 97%, 98% or 99% identical to a polynucleotide selected from the polynucleotides provided herein.

In certain embodiments, a polynucleotide is isolated. In certain embodiments, a polynucleotide is substantially pure.

Vectors and cells comprising the polynucleotides described herein are also provided. In some embodiments of each or any of the above- or below-mentioned embodiments, an expression vector comprises a polynucleotide molecule. In some embodiments of each or any of the above- or below-mentioned embodiments, a host cell comprises an expression vector comprising the polynucleotide molecule. In some embodiments of each or any of the above- or below-mentioned embodiments, a host cell comprises one or more expression vectors comprising polynucleotide molecules. In some embodiments of each or any of the above- or below-mentioned embodiments, a host cell comprises a polynucleotide molecule. In some embodiments of each or any of the above- or below-mentioned embodiments, a host cell comprises one or more polynucleotide molecules.

### 5.4 Constant Region Libraries

In another aspect, provided herein is an antibody constant region library comprising plural molecules each comprising at least one engineered antibody constant region variant (e.g., a CH1 region variant and/or a CL region variant), and the constant region variants in the plural molecules in the library comprise a variety of loop regions. In some embodiments of each or any of the above- or below-mentioned embodiments, the library diversity may range from ˜10⁷-10¹⁶ (for a single loop) to ˜10¹⁸-10³³ (for double loops). In some embodiments of each or any of the above- or below-mentioned embodiments, the library diversity may range from ˜10⁶-10⁷ for a single loop. In some embodiments, the library diversity may range from ˜10⁷-10⁸ for a single loop. In some embodiments of each or any of the above- or below-mentioned embodiments, the library diversity may range from ˜10⁸-10⁹ for a single loop. In some embodiments of each or any of the above- or below-mentioned embodiments, the library diversity may range from ˜10⁹-10¹⁰ for a single loop. In some embodiments of each or any of the above- or below-mentioned embodiments, the library diversity may range from ˜10¹⁰-10¹¹ for a single loop. In some embodiments of each or any of the above- or below-mentioned embodiments, the library diversity may range from ˜10¹¹-10¹² for a single loop. In some embodiments of each or any of the above- or below-mentioned embodiments, the library diversity may range from ˜10¹²-10¹³ for a single loop. In some embodiments of each or any of the above- or below-mentioned embodiments, the library diversity may range from ˜10¹³-10¹⁴ for a single loop. In some embodiments of each or any of the above- or below-mentioned embodiments, the library diversity may range from ˜10¹⁴-10¹⁵ for a single loop. In some embodiments of each or any of the above- or below-mentioned embodiments, the library diversity may range from ˜10¹⁵-10¹⁶ for a single loop. In some embodiments of each or any of the above- or below-mentioned embodiments, the library diversity may range from ˜10¹⁶-10¹⁷ for a single loop.

The present library can be constructed by introducing various amino acid sequences into at least one of the loop regions in an antibody constant region (e.g., a CH1 region or a CL region), and optionally replacing one or more amino acid residues within the loop region in the antibody constant region. For example, in some embodiments of each or any of the above- or below-mentioned embodiments, the library provided herein comprises a plural molecules with different amino acid sequences that are introduced to and/or replace a region within AB loop of an antibody constant region (e.g., a CH1 region or a CL region). In some embodiments of each or any of the above- or below-mentioned embodiments, the library provided herein comprises a plural molecules with different amino acid sequences that are introduced to and/or replace a region within BC loop of an antibody constant region (e.g., a CH1 region or a CL region). In some embodiments of each or any of the above- or below-mentioned embodiments, the library provided herein comprises a plural molecules with different amino acid sequences that are introduced to and/or replace a region within CD loop of an antibody constant region (e.g., a CH1 region or a CL region). In some embodiments of each or any of the above- or below-mentioned embodiments, the library provided herein comprises a plural molecules with different amino acid sequences that are introduced to and/or replace a region within DE loop of an antibody constant region (e.g., a CH1 region or a CL region). In some embodiments of each or any of the above- or below-mentioned embodiments, the library provided herein comprises a plural molecules with different amino acid sequences that are introduced to and/or replace a region within EF loop of an antibody constant region (e.g., a CH1 region or a CL region). In some embodiments of each or any of the above- or below-mentioned embodiments, the library provided herein comprises a plural molecules with different amino acid sequences that are introduced to and/or replace a region within FG loop of an antibody constant region (e.g., a CH1 region or a CL region).

In some embodiments of each or any of the above- or below-mentioned embodiments, various amino acid sequences are introduced to and/or replace an amino acid fragment within the AB, BC, CD, DE, EF, and/or FG loop regions of the CH1 region. In some embodiments of each or any of the above- or below-mentioned embodiments, various amino acid sequences are introduced to and/or replace an amino acid fragment within the AB, BC, CD, DE, EF, and/or FG loop regions of the CL region. In some embodiments of each or any of the above- or below-mentioned embodiments, various amino acid sequences are introduced to and/or replace an amino acid fragment outside of the AB, BC, CD, DE, EF, and/or FG loop regions of the CH1 region. In some embodiments of each or any of the above- or below-mentioned embodiments, various amino acid sequences are introduced to and/or replace an amino acid fragment outside of the AB, BC, CD, DE, EF, and/or FG loop regions of the CL region. In some embodiments of each or any of the above- or below-mentioned embodiments, various amino acid sequences are introduced to and/or replace an amino acid fragment within the A, B, C, D, E, and/or F β-strands regions of the CH1 region. In some embodiments of each or any of the above- or below-mentioned embodiments, various amino acid sequences are introduced to and/or replace an amino acid fragment within the A, B, C, D, E, and/or F β-strands regions of the CL region.

In some embodiments of each or any of the above- or below-mentioned embodiments, various amino acid sequences can be inserted in a loop region. Various amino acid sequences can be inserted in AB loop region of CH1 or CL region. Various amino acid sequences can be inserted in BC loop region of CH1 or CL region. Various amino acid sequences can be inserted in CD loop region of CH1 or CL region. Various amino acid sequences can be inserted in DE loop region of CH1 or CL region. Various amino acid sequences can be inserted in EF loop region of CH1 or CL region. Various amino acid sequences can be inserted in FG loop region of CH1 or CL region.

In other embodiments, various amino acid sequences can replace a region within a loop region. Various amino acid sequences can replace a region within AB loop region of CH1 or CL region. Various amino acid sequences can replace a region within BC loop region of CH1 or CL region. Various amino acid sequences can replace a region within CD loop region of CH1 or CL region. Various amino acid sequences can replace a region within DE loop region of CH1 or CL region. Various amino acid sequences can replace a region within EF loop region of CH1 or CL region. Various amino acid sequences can replace a region within FG loop region of CH1 or CL region.

In some embodiments of each or any of the above- or below-mentioned embodiments, the molecules in the library provided herein comprise diverse sequences in one loop region. In other embodiments, the molecules in the library provided herein comprise diverse sequences in two or more loop regions within one or more constant regions.

Thus, in some embodiments of each or any of the above- or below-mentioned embodiments, provided herein is a Fab constant region library (CRL) comprising a population of molecules each comprising a region derived from a CH1 region and/or a region derived from a CL region of an antibody, wherein the population of the molecules comprise diverse amino acid sequences in the region derived from the CH1 region and/or the region derived from the CL region. In some embodiments of each or any of the above- or below-mentioned embodiments, the diverse amino acid sequences in the region derived from the CH1 region are at the AB, BC, CD, DE, EF, and/or FG loop regions of the CH1 region. In some embodiments of each or any of the above- or below-mentioned embodiments, the diverse amino acid sequences in the region derived from the CL region are at the AB, BC, CD, DE, EF, and/or FG loop regions of the CL region. In some embodiments of each or any of the above- or below-mentioned embodiments, the diverse amino acid sequences in the region derived from the CH1 region are outside of the AB, BC, CD, DE, EF, and/or FG loop regions of the CH1 region. In some embodiments of each or any of the above- or below-mentioned embodiments, the diverse amino acid sequences in the region derived from the CL region are outside of the AB, BC, CD, DE, EF, and/or FG loop regions of the CL region. In some embodiments of each or any of the above- or below-mentioned embodiments, the diverse amino acid sequences in the region derived from the CH1 region are at the A, B, C, D, E, and/or F β-strands regions of the CH1 region. In some embodiments of each or any of the above- or below-mentioned embodiments, the diverse amino acid sequences in the region derived from the CL region are at the A, B, C, D, E, and/or F β-strands regions of the CL region.

In some embodiments of each or any of the above- or below-mentioned embodiments, the population of the molecules comprise diverse amino acid sequences in one or two loop region(s) in the region derived from the CH1 region. In some embodiments of each or any of the above- or below-mentioned embodiments, the population of the molecules comprise diverse amino acid sequences in one or two loop region(s) in the region derived from the CL region. In some embodiments of each or any of the above- or below-mentioned embodiments, the population of the molecules comprise diverse amino acid sequences at the CD loop region of the CH1 region. In some embodiments of each or any of the above- or below-mentioned embodiments, the population of the molecules comprise diverse amino acid sequences at the DE loop region of the CH1 region. In some embodiments of each or any of the above- or below-mentioned embodiments, the population of the molecules comprise diverse amino acid sequences at the CD loop region and the DE loop region of the CH1 region. In some embodiments of each or any of the above- or below-mentioned embodiments, the population of the molecules comprise diverse amino acid sequences at the CD loop region of the CL region. In some embodiments of each or any of the above- or below-mentioned embodiments, the population of the molecules comprise diverse amino acid sequences at the DE loop region of the CL region. In some embodiments of each or any of the above- or below-mentioned embodiments, the population of the molecules comprise diverse amino acid sequences at the CD loop region and the DE loop region of the CL region. In some embodiments of each or any of the above- or below-mentioned embodiments, the population of the molecules comprise diverse amino acid sequences at the CD loop region of the CH1 region; and the population of the molecules comprise diverse amino acid sequences at the CD loop region of the CL region. In some embodiments of each or any of the above- or below-mentioned embodiments, the population of the molecules comprise diverse amino acid sequences at the CD loop region of the CH1 region; and the population of the molecules comprise diverse amino acid sequences at the DE loop region of the CL region. In some embodiments of each or any of the above- or below-mentioned embodiments, the population of the molecules comprise diverse amino acid sequences at the CD loop region of the CH1 region; and the population of the molecules comprise diverse amino acid sequences at the CD loop region and the DE loop region of the CL region. In some embodiments of each or any of the above- or below-mentioned embodiments, the population of the molecules comprise diverse amino acid sequences at the DE loop region of the CH1 region; and the population of the molecules comprise diverse amino acid sequences at the CD loop region of the CL region. In some embodiments of each or any of the above- or below-mentioned embodiments, the population of the molecules comprise diverse amino acid sequences at the DE loop region of the CH1 region; and the population of the molecules comprise diverse amino acid sequences at the DE loop region of the CL region. In some embodiments of each or any of the above- or below-mentioned embodiments, the population of the molecules comprise diverse amino acid sequences at the DE loop region of the CH1 region; and the population of the molecules comprise diverse amino acid sequences at the CD loop region and the DE loop region of the CL region. In some embodiments of each or any of the above- or below-mentioned embodiments, the population of the molecules comprise diverse amino acid sequences at the CD loop region of the CH1 region and the DE loop region of the CH1 region; and the population of the molecules comprise diverse amino acid sequences at the CD loop region of the CL region. In some embodiments of each or any of the above- or below-mentioned embodiments, the population of the molecules comprise diverse amino acid sequences at the CD loop region and the DE loop region of the CH1 region; and the population of the molecules comprise diverse amino acid sequences at the DE loop region of the CL region. In some embodiments of each or any of the above- or below-mentioned embodiments, the population of the molecules comprise diverse amino acid sequences at the CD loop region and the DE loop region of the CH1 region; and the population of the molecules comprise diverse amino acid sequences at the CD loop region and the DE loop region of the CL region.

In some specific embodiments, the region derived from the CH1 region is a region derived from a human IgG1 CH1 region comprising an amino acid sequence of SEQ ID NO: 1, and wherein the region derived from the CH1 region comprises an amino acid sequence having at least 70%, 75%, 80%, 85%, 90% or 95% identity to SEQ ID NO:1. In some specific embodiments, the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2. In some specific embodiments, the region derived from the CL region is a region derived from a human CL lambda region comprising an amino acid sequence of SEQ ID NO:3, and wherein the region derived from the CL region comprises an amino acid sequence having at least 70%, 75%, 80%, 85%, 90% or 95% identity to SEQ ID NO:3.

In some more specific embodiments, the amino acid residues TSG (EU numbering 164-166) of the CD loop of the human IgG1 CH1 region are replaced with diverse amino acid sequences in the molecules in the Fab CRL. In some more specific embodiments, the amino acid residue S (EU numbering 165) of the CD loop of the human IgG1 CH1 region is replaced with diverse amino acid sequences in the molecules in the Fab CRL. In some more specific embodiments, the amino acid residues QSS (EU numbering 175-177) of the DE loop of the human IgG1 CH1 region are replaced with diverse amino acid sequences in the molecules in the Fab CRL. In some more specific embodiments, the amino acid residues SGNS (EU numbering 156-159) of the CD loop of the human CL kappa region are replaced with diverse amino acid sequences in the molecules in the Fab CRL. In some more specific embodiments, the amino acid residues SKD (EU numbering 168-170) of the DE loop of the human CL kappa region are replaced with diverse amino acid sequences in the molecules in the Fab CRL. In some more specific embodiments, the amino acid residue K (EU numbering 169) of the DE loop of the human CL kappa region is replaced with diverse amino acid sequences in the molecules in the Fab CRL.

The diverse amino acid sequences introduced into one loop region in the population of the molecules in the present library can be in various length or with the same length but different sequences. In some embodiments of each or any of the above- or below-mentioned embodiments, the diverse amino acid sequences in one loop comprise 7 to 15 amino acid residues. In some embodiments of each or any of the above- or below-mentioned embodiments, the diverse amino acid sequences in one loop comprise 7 amino acid residues. In some embodiments of each or any of the above- or below-mentioned embodiments, the diverse amino acid sequences in one loop comprise 8 amino acid residues. In some embodiments of each or any of the above- or below-mentioned embodiments, the diverse amino acid sequences in one loop comprise 9 amino acid residues. In some embodiments of each or any of the above- or below-mentioned embodiments, the diverse amino acid sequences in one loop comprise 10 amino acid residues. In some embodiments of each or any of the above- or below-mentioned embodiments, the diverse amino acid sequences in one loop comprise 11 amino acid residues. In some embodiments of each or any of the above- or below-mentioned embodiments, the diverse amino acid sequences in one loop comprise 12 amino acid residues. In some embodiments of each or any of the above- or below-mentioned embodiments, the diverse amino acid sequences in one loop comprise 13 amino acid residues. In some embodiments of each or any of the above- or below-mentioned embodiments, the diverse amino acid sequences in one loop comprise 14 amino acid residues. In some embodiments of each or any of the above- or below-mentioned embodiments, the diverse amino acid sequences in one loop comprise 15 amino acid residues. In some embodiments of each or any of the above- or below-mentioned embodiments, the diverse amino acid sequences in one loop comprise more than 15 amino acid residues, such as 16, 17, 18, 19, 20 or more amino acid residues.

In addition to various constant region variants, the molecules in the present library may further comprise additional domains such as a VH region and/or a VL region. In some embodiments of each or any of the above- or below-mentioned embodiments, each of the molecules in the library further comprise a VH region and a VL region. Thus, in some embodiments of each or any of the above- or below-mentioned embodiments, provided herein is a Fab constant region library (CRL) comprising a population of binding molecules, wherein each of the binding molecules comprises (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the population of the binding molecules comprise diverse amino acid sequences in the region derived from the CH1 region and/or the region derived from the CL region. In some specific embodiments, the molecules in the present library are Fab fragments. In some embodiments of each or any of the above- or below-mentioned embodiments, the library provided herein can be constructed as described in Section 7 below.

Moreover, molecules in any antibody format can be used to construct the present Fab CRL. In some embodiments of each or any of the above- or below-mentioned embodiments, diverse constant regions (e.g., diverse CH1 regions and/or CL regions) are introduced into intact antibodies to generate the present library. Antibodies can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein is an IgG antibody, such as an IgG1 antibody, IgG2 antibody or IgG4 antibody (e.g., IgG4 nullbody and variants of IgG4 antibodies). In a specific embodiment, the IgG antibody is an IgG1 antibody. In other embodiments, diverse constant regions (e.g., diverse CH1 regions and/or CL regions) are introduced into intact antibody fragments to generate the present library. Exemplary fragments include but not limited to a Fab, a Fab′, a F(ab′)2, a bispecific Fab, a single chain Fab. The antibodies may be from or derived from any animal origin including birds and mammals (e.g., human, monkey, murine, donkey, sheep, rabbit, goat, guinea pig, camel, horse, or chicken). In certain embodiments, the antibodies provided herein are human or humanized monoclonal antibodies. As used herein, “human” antibodies include antibodies having the amino acid sequence of a human immunoglobulin and include antibodies isolated from human immunoglobulin libraries or from mice that express antibodies from human genes. In certain embodiments, the antibodies are full mouse antibodies. In certain embodiments, the antibodies are mouse-human chimeric antibodies. In certain embodiments, the antibodies are humanized antibodies. In certain embodiments, the antibodies are fully human antibodies. In other embodiments, the antibodies provided herein are humanized antibodies (e.g., comprising human constant and framework regions). In some embodiments of each or any of the above- or below-mentioned embodiments, the antibody is a bispecific antibody. In some embodiments of each or any of the above- or below-mentioned embodiments, the antibody is a trispecific antibody. In some embodiments of each or any of the above- or below-mentioned embodiments, the antibody is a quadraspecific antibody. In some embodiments of each or any of the above- or below-mentioned embodiments, the antibody is a bivalent antibody. In some embodiments of each or any of the above- or below-mentioned embodiments, the antibody is a trivalent antibody. In some embodiments of each or any of the above- or below-mentioned embodiments, the antibody is a quadravalent antibody.

In yet another aspect, provided herein is a method of producing a binding molecule comprising a first step for performing a function of identifying an antibody constant region variant capable of binding to an antigen; and a second step of constructing the binding molecule that comprises the antibody constant region variant. In some embodiments of each or any of the above- or below-mentioned embodiments, the first step comprises screening the Fab CRL provided herein. In some embodiments of each or any of the above- or below-mentioned embodiments, provided herein is a method for identifying a binding molecule comprising a first binding domain that binds to a first antigen and a second binding domain that binds to a second antigen, comprising screening the Fab CRL provided herein for identifying the binding molecule that binds to the second antigen with a higher affinity than a reference level, wherein the first binding domain comprises the VH region and the VL region of an antibody, and wherein the second binding domain comprises an antibody constant region variant. Screening of the libraries can be accomplished by various techniques known in the art. For example, a specific antigen (e.g., polypeptide, fragment, or epitope of the antigen) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

In yet another aspect, provided herein is a binding molecule produced according the method provided herein using a Fab CRL.

### 5.5 Methods or Processes of Making the Antibodies

In yet another aspect, provided herein are methods or processes for making the various molecules provided herein. In some embodiments, provided herein is a process for making a molecule that binds to more than one target molecule, comprising: a step for performing a function of obtaining a binding domain capable of binding to a first antigen; a step for performing a function of obtaining a binding domain capable of binding to a second antigen; and a step for performing a function of providing a molecule capable of binding to the first antigen and the second antigen.

Recombinant expression of an antibody provided herein requires construction of an expression vector containing a polynucleotide that encodes the antibody or antigen binding fragment thereof. Once a polynucleotide encoding an antibody molecule, heavy or light chain of an antibody, or fragment thereof (such as, but not necessarily, containing the heavy and/or light chain variable domain) provided herein has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule provided herein or a fragment thereof, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody provided herein. Thus, also provided herein are host cells containing a polynucleotide encoding an antibody provided herein or fragments thereof, or a heavy or light chain thereof, or fragment thereof, or a single chain antibody provided herein, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In some embodiments of each or any of the above- or below-mentioned embodiments, antibodies provided herein are produced in CHO cells. In a specific embodiment, the expression of nucleotide sequences encoding antibodies provided herein which immunospecifically bind to a specific antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. In some embodiments of each or any of the above- or below-mentioned embodiments, fully human monoclonal antibodies provided herein are produced in mammalian cells, such as CHO cells.

For long-term, high-yield production of recombinant proteins, stable expression can be utilized. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two or more expression vectors provided herein. The two or more vectors may contain identical selectable markers which enable equal expression of, e.g., heavy and light chain polypeptides. Alternatively, a single vector may be used which encodes, and is capable of expressing different component polypeptides of the present antibodies, e.g., both heavy and light chain polypeptides. The coding sequences may comprise cDNA or genomic DNA.

Once an antibody molecule provided herein has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies provided herein can be fused to heterologous polypeptide sequences described herein or otherwise known in the art to facilitate purification.

### 5.6 Pharmaceutical Compositions

In one aspect, the present disclosure further provides pharmaceutical compositions comprising at least one antibody or antigen binding fragment thereof of the present disclosure. In some embodiments of each or any of the above- or below-mentioned embodiments, a pharmaceutical composition comprises therapeutically effective amount of an antibody or antigen binding fragment thereof provided herein and a pharmaceutically acceptable excipient.

Pharmaceutical compositions comprising an antibody or antigen binding fragment thereof are prepared for storage by mixing the protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.7 Methods of Using

In yet another aspect, provided herein is a method of enriching, isolating, separating, purifying, sorting, selecting, capturing, detecting or depleting cells expressing a specific antigen, comprising providing a sample comprising the cells expressing a specific antigen; contacting the sample with a multispecific antibody; and enriching, isolating, separating, purifying, sorting, selecting, capturing, detecting or depleting the cells expressing a specific antigen that binds to the multispecific antibody, wherein the multispecific antibody comprises a first binding domain capable of binding to a first antigen, and a second binding domain capable of binding to a second antigen. In some embodiments of each or any of the above- or below-mentioned embodiments, the sample is a blood sample. In some embodiments of each or any of the above- or below-mentioned embodiments, the sample is a tissue sample.

In yet another aspect, provided herein is a method of treating cancer in a subject, comprising administering to the subject an antibody or antibodies described in any one of the above or below-mentioned embodiments provided herein. In some embodiments of each or any of the above- or below-mentioned embodiments, the cancer is a solid tumor cancer. In some embodiments of each or any of the above- or below-mentioned embodiments, the cancer is a blood cancer.

In another aspect, provided herein is a method of treating a disease or disorder in a subject comprising administering to the subject an effective amount of an antibody or antigen binding fragment thereof provided herein.

Also provided herein is a method of treatment of a disease or disorder, wherein the subject is administered one or more therapeutic agents in combination with the antibody or antigen-binding fragment thereof provided herein.

In another aspect, provided herein is the use of the antibody or antigen binding fragment thereof provided herein in the manufacture of a medicament for treating a disease or disorder in a subject.

In another aspect, provided herein is the use of a pharmaceutical composition provided herein in the manufacture of a medicament for treating a disease or disorder in a subject.

In a specific embodiment, provided herein is a composition for use in the prevention and/or treatment of a disease or condition comprising an antibody or antigen binding fragment thereof provided herein. In one embodiment, provided herein is a composition for use in the prevention of a disease or condition, wherein the composition comprises an antibody or antigen binding fragment thereof provided herein. In one embodiment, provided herein is a composition for use in the treatment of a disease or condition, wherein the composition comprises an antibody or antigen binding fragment thereof provided herein. In certain embodiments, the subject is a subject in need thereof. In some embodiments of each or any of the above- or below-mentioned embodiments, the subject has the disease or condition. In other embodiments, the subject is at risk of having the disease or condition. In some embodiments of each or any of the above- or below-mentioned embodiments, the administration results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided herein. In one embodiment, provided herein is a composition for use in the prevention of a symptom of a disease or condition, wherein the composition comprises an antibody or antigen binding fragment thereof provided herein. In one embodiment, provided herein is a composition for use in the treatment of a symptom of a disease or condition, wherein the composition comprises an antibody or antigen binding fragment thereof provided herein. In certain embodiments, the subject is a subject in need thereof. In some embodiments of each or any of the above- or below-mentioned embodiments, the subject has the disease or condition. In other embodiments, the subject is at risk of having the disease or condition. In some embodiments of each or any of the above- or below-mentioned embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein. In certain embodiments, the subject is a subject in need thereof. In some embodiments of each or any of the above- or below-mentioned embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In certain embodiments, the subject is a subject in need thereof. In some embodiments of each or any of the above- or below-mentioned embodiments, the subject has the disease or condition. In other embodiments, the subject is at risk of having the disease or condition. In some embodiments of each or any of the above- or below-mentioned embodiments, the administration results in the prevention or treatment of the symptom of the disease or condition.

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. Also provided herein, in some embodiments, are uses of the antibodies or antigen binding fragments provided herein for preventing and/or treating a disease or condition. Also provided herein, in some embodiments, are the antibodies or antigen binding fragments provided herein for use in preventing and/or treating a disease or condition. Also provided herein, in some embodiments, uses of the antibodies or antigen binding fragments provided herein for the manufacture of a medicament for preventing and/or treating a disease or condition. In one aspect, the antibody or antigen binding fragment thereof is substantially purified (i.e., substantially free from substances that limit its effect or produce undesired side-effects). The subject administered a therapy can be a mammal such as non-primate or a primate (e.g., a human). In a one embodiment, the subject is a human. In another embodiment, the subject is a human with a disease or condition.

In some embodiments of each or any of the above- or below-mentioned embodiments, the present binding molecules are used for treating solid tumor cancer. In other embodiments, the present binding molecules are used for treating blood cancer. In other embodiments, the disease or disorder is an autoimmune and inflammatory disease. In other embodiments, the disease or disorder is an infectious disease.

In some embodiments of each or any of the above- or below-mentioned embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments of each or any of the above- or below-mentioned embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments of each or any of the above- or below-mentioned embodiments, the disease or disorder is a hematological cancer, such as leukemia, lymphoma, or myeloma. In some embodiments of each or any of the above- or below-mentioned embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments of each or any of the above- or below-mentioned embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments of each or any of the above- or below-mentioned embodiments, the cancer is an adrenal cancer. In some embodiments of each or any of the above- or below-mentioned embodiments, the cancer is an anal cancer. In some embodiments of each or any of the above- or below-mentioned embodiments, the cancer is an appendix cancer. In some embodiments of each or any of the above- or below-mentioned embodiments, the cancer is a bile duct cancer. In some embodiments of each or any of the above- or below-mentioned embodiments, the cancer is a bladder cancer. In some embodiments of each or any of the above- or below-mentioned embodiments, the cancer is a bone cancer. In some embodiments of each or any of the above- or below-mentioned embodiments, the cancer is a brain cancer. In some embodiments of each or any of the above- or below-mentioned embodiments, the cancer is a breast cancer. In some embodiments of each or any of the above- or below-mentioned embodiments, the cancer is a cervical cancer. In some embodiments of each or any of the above- or below-mentioned embodiments, the cancer is a colorectal cancer. In some embodiments of each or any of the above- or below-mentioned embodiments, the cancer is an esophageal cancer. In some embodiments of each or any of the above- or below-mentioned embodiments, the cancer is a gallbladder cancer. In some embodiments of each or any of the above- or below-mentioned embodiments, the cancer is a gestational trophoblastic. In some embodiments of each or any of the above- or below-mentioned embodiments, the cancer is a head and neck cancer. In some embodiments of each or any of the above- or below-mentioned embodiments, the cancer is a Hodgkin lymphoma. In some embodiments of each or any of the above- or below-mentioned embodiments, the cancer is an intestinal cancer. In some embodiments of each or any of the above- or below-mentioned embodiments, the cancer is a kidney cancer. In some embodiments of each or any of the above- or below-mentioned embodiments, the cancer is a leukemia. In some embodiments of each or any of the above- or below-mentioned embodiments, the cancer is a liver cancer. In some embodiments of each or any of the above- or below-mentioned embodiments, the cancer is a lung cancer. In some embodiments of each or any of the above- or below-mentioned embodiments, the cancer is a melanoma. In some embodiments of each or any of the above- or below-mentioned embodiments, the cancer is a mesothelioma. In some embodiments of each or any of the above- or below-mentioned embodiments, the cancer is a multiple myeloma (MM). In some embodiments of each or any of the above- or below-mentioned embodiments, the cancer is a neuroendocrine tumor. In some embodiments of each or any of the above- or below-mentioned embodiments, the cancer is a non-Hodgkin lymphoma. In some embodiments of each or any of the above- or below-mentioned embodiments, the cancer is an oral cancer. In some embodiments of each or any of the above- or below-mentioned embodiments, the cancer is an ovarian cancer. In some embodiments of each or any of the above- or below-mentioned embodiments, the cancer is a pancreatic cancer. In some embodiments of each or any of the above- or below-mentioned embodiments, the cancer is a prostate cancer. In some embodiments of each or any of the above- or below-mentioned embodiments, the cancer is a sinus cancer. In some embodiments of each or any of the above- or below-mentioned embodiments, the cancer is a skin cancer. In some embodiments of each or any of the above- or below-mentioned embodiments, the cancer is a soft tissue sarcoma spinal cancer. In some embodiments of each or any of the above- or below-mentioned embodiments, the cancer is a stomach cancer. In some embodiments of each or any of the above- or below-mentioned embodiments, the cancer is a testicular cancer. In some embodiments of each or any of the above- or below-mentioned embodiments, the cancer is a throat cancer. In some embodiments of each or any of the above- or below-mentioned embodiments, the cancer is a thyroid cancer. In some embodiments of each or any of the above- or below-mentioned embodiments, the cancer is a uterine cancer endometrial cancer. In some embodiments of each or any of the above- or below-mentioned embodiments, the cancer is a vaginal cancer. In some embodiments of each or any of the above- or below-mentioned embodiments, the cancer is a vulvar cancer.

In some embodiments of each or any of the above- or below-mentioned embodiments, the adrenal cancer is an adrenocortical carcinoma (ACC), adrenal cortex cancer, pheochromocytoma, or neuroblastoma. In some embodiments of each or any of the above- or below-mentioned embodiments, the anal cancer is a squamous cell carcinoma, cloacogenic carcinoma, adenocarcinoma, basal cell carcinoma, or melanoma. In some embodiments of each or any of the above- or below-mentioned embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments of each or any of the above- or below-mentioned embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments of each or any of the above- or below-mentioned embodiments, the bladder cancer is transitional cell carcinoma (TCC), papillary carcinoma, flat carcinoma, squamous cell carcinoma, adenocarcinoma, small-cell carcinoma, or sarcoma. In some embodiments of each or any of the above- or below-mentioned embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments of each or any of the above- or below-mentioned embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments of each or any of the above- or below-mentioned embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments of each or any of the above- or below-mentioned embodiments, the cervical cancer is a squamous cell carcinoma, or adenocarcinoma. In some embodiments of each or any of the above- or below-mentioned embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments of each or any of the above- or below-mentioned embodiments, the esophageal cancer is an adenocarcinoma or squamous cell carcinoma. In some embodiments of each or any of the above- or below-mentioned embodiments, the gall bladder cancer is an adenocarcinoma, papillary adenocarcinoma, adenosquamous carcinoma, squamous cell carcinoma, small cell carcinoma, or sarcoma. In some embodiments of each or any of the above- or below-mentioned embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments of each or any of the above- or below-mentioned embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments of each or any of the above- or below-mentioned embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments of each or any of the above- or below-mentioned embodiments, the intestinal cancer is a small intestine cancer, small bowel cancer, adenocarcinoma, sarcoma, gastrointestinal stromal tumors, carcinoid tumors, or lymphoma. In some embodiments of each or any of the above- or below-mentioned embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments of each or any of the above- or below-mentioned embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments of each or any of the above- or below-mentioned embodiments, the liver cancer is a hepatocellular carcinoma (HCC), fibrolamellar HCC, cholangiocarcinoma, angiosarcoma, or liver metastasis. In some embodiments of each or any of the above- or below-mentioned embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments of each or any of the above- or below-mentioned embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments of each or any of the above- or below-mentioned embodiments, the mesothelioma is a pleural mesothelioma, peritoneal mesothelioma, pericardial mesothelioma, or testicular mesothelioma. In some embodiments of each or any of the above- or below-mentioned embodiments, the multiple myeloma is an active myeloma or smoldering myeloma. In some embodiments of each or any of the above- or below-mentioned embodiments, the neuroendocrine tumor is a gastrointestinal neuroendocrine tumor, pancreatic neuroendocrine tumor, or lung neuroendocrine tumor. In some embodiments of each or any of the above- or below-mentioned embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments of each or any of the above- or below-mentioned embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments of each or any of the above- or below-mentioned embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments of each or any of the above- or below-mentioned embodiments, the pancreatic cancer is a pancreatic exocrine gland cancer, pancreatic endocrine gland cancer, or pancreatic adenocarcinoma, islet cell tumor, or neuroendocrine tumor. In some embodiments of each or any of the above- or below-mentioned embodiments, the prostate cancer is a prostate adenocarcinoma, prostate sarcoma, transitional cell carcinoma, small cell carcinoma, or neuroendocrine tumor. In some embodiments of each or any of the above- or below-mentioned embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments of each or any of the above- or below-mentioned embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments of each or any of the above- or below-mentioned embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments of each or any of the above- or below-mentioned embodiments, the spinal cancer is a spinal metastatic tumor. In some embodiments of each or any of the above- or below-mentioned embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments of each or any of the above- or below-mentioned embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments of each or any of the above- or below-mentioned embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments of each or any of the above- or below-mentioned embodiments, the thyroid cancer is a papillary carcinoma, follicular carcinoma, Hürthle cell carcinoma, medullary thyroid carcinoma, or anaplastic carcinoma. In some embodiments of each or any of the above- or below-mentioned embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments of each or any of the above- or below-mentioned embodiments, the vaginal cancer is a squamous cell carcinoma, adenocarcinoma, melanoma, or sarcoma. In some embodiments of each or any of the above- or below-mentioned embodiments, the vulvar cancer is a squamous cell carcinoma or adenocarcinoma.

In some embodiments of each or any of the above- or below-mentioned embodiments, the disease or disorder is caused by a pathogen. In some embodiments of each or any of the above- or below-mentioned embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments of each or any of the above- or below-mentioned embodiments, the infectious disease is Acute Flaccid Myelitis (AFM). In some embodiments of each or any of the above- or below-mentioned embodiments, the infectious disease is Anaplasmosis. In some embodiments of each or any of the above- or below-mentioned embodiments, the infectious disease is Anthrax. In some embodiments of each or any of the above- or below-mentioned embodiments, the infectious disease is Babesiosis. In some embodiments of each or any of the above- or below-mentioned embodiments, the infectious disease is Botulism. In some embodiments of each or any of the above- or below-mentioned embodiments, the infectious disease is Brucellosis. In some embodiments of each or any of the above- or below-mentioned embodiments, the infectious disease is Campylobacteriosis. In some embodiments of each or any of the above- or below-mentioned embodiments, the infectious disease is Carbapenem-resistant Infection. In some embodiments of each or any of the above- or below-mentioned embodiments, the infectious disease is Chancroid. In some embodiments of each or any of the above- or below-mentioned embodiments, the infectious disease is Chikungunya Virus Infection. In some embodiments of each or any of the above- or below-mentioned embodiments, the infectious disease is Chlamydia. In some embodiments of each or any of the above- or below-mentioned embodiments, the infectious disease is Ciguatera. In some embodiments of each or any of the above- or below-mentioned embodiments, the infectious disease is Difficile Infection. In some embodiments of each or any of the above- or below-mentioned embodiments, the infectious disease is Perfringens. In some embodiments of each or any of the above- or below-mentioned embodiments, the infectious disease is Coccidioidomycosis fungal infection. In some embodiments of each or any of the above- or below-mentioned embodiments, the infectious disease is coronavirus. In some embodiments of each or any of the above- or below-mentioned embodiments, the infectious disease is Covid-19 (SARS-COV-2). In some embodiments of each or any of the above- or below-mentioned embodiments, the infectious disease is Creutzfeldt-Jacob Disease/transmissible spongiform encephalopathy. In some embodiments of each or any of the above- or below-mentioned embodiments, the infectious disease is Cryptosporidiosis (Crypto). In some embodiments of each or any of the above- or below-mentioned embodiments, the infectious disease is Cyclosporiasis. In some embodiments of each or any of the above- or below-mentioned embodiments, the infectious disease is Dengue 1, 2, 3 or 4. In some embodiments of each or any of the above- or below-mentioned embodiments, the infectious disease is Diphtheria. In some embodiments of each or any of the above- or below-mentioned embodiments, the infectious disease is E. coli infection/Shiga toxin-producing (STEC). In some embodiments of each or any of the above- or below-mentioned embodiments, the infectious disease is Eastern Equine Encephalitis. In some embodiments of each or any of the above- or below-mentioned embodiments, the infectious disease is Hemorrhagic Fever (Ebola). In some embodiments of each or any of the above- or below-mentioned embodiments, the infectious disease is Ehrlichiosis. In some embodiments of each or any of the above- or below-mentioned embodiments, the infectious disease is Encephalitis. In some embodiments of each or any of the above- or below-mentioned embodiments, the infectious disease is Arboviral or parainfectious. In some embodiments of each or any of the above- or below-mentioned embodiments, the infectious disease is Non-Polio Enterovirus. In some embodiments of each or any of the above- or below-mentioned embodiments, the infectious disease is D68 Enteroviru(EV-D68). In some embodiments of each or any of the above- or below-mentioned embodiments, the infectious disease is Giardiasis. In some embodiments of each or any of the above- or below-mentioned embodiments, the infectious disease is Glanders. In some embodiments of each or any of the above- or below-mentioned embodiments, the infectious disease is Gonococcal Infection. In some embodiments of each or any of the above- or below-mentioned embodiments, the infectious disease is Granuloma inguinale. In some embodiments of each or any of the above- or below-mentioned embodiments, the infectious disease is Haemophilus Influenza disease Type B (Hib or H-flu). In some embodiments of each or any of the above- or below-mentioned embodiments, the infectious disease is Hantavirus Pulmonary Syndrome (HPS). In some embodiments of each or any of the above- or below-mentioned embodiments, the infectious disease is Hemolytic Uremic Syndrome (HUS). In some embodiments of each or any of the above- or below-mentioned embodiments, the infectious disease is Hepatitis A (Hep A). In some embodiments of each or any of the above- or below-mentioned embodiments, the infectious disease is Hepatitis B (Hep B). In some embodiments of each or any of the above- or below-mentioned embodiments, the infectious disease is Hepatitis C (Hep C). In some embodiments of each or any of the above- or below-mentioned embodiments, the infectious disease is Hepatitis D (Hep D). In some embodiments of each or any of the above- or below-mentioned embodiments, the infectious disease is Hepatitis E (Hep E). In some embodiments of each or any of the above- or below-mentioned embodiments, the infectious disease is Herpes. In some embodiments of each or any of the above- or below-mentioned embodiments, the infectious disease is Herpes Zoster (Shingles). In some embodiments of each or any of the above- or below-mentioned embodiments, the infectious disease is Histoplasmosis infection. In some embodiments of each or any of the above- or below-mentioned embodiments, the infectious disease is Human Immunodeficiency Virus/AIDS (HIV/AIDS). In some embodiments of each or any of the above- or below-mentioned embodiments, the infectious disease is Human Papillomavirus (HPV). In some embodiments of each or any of the above- or below-mentioned embodiments, the infectious disease is Influenza (Flu). In some embodiments of each or any of the above- or below-mentioned embodiments, the infectious disease is Legionellosis (Legionnaires Disease). In some embodiments of each or any of the above- or below-mentioned embodiments, the infectious disease is Leprosy (Hansens Disease). In some embodiments of each or any of the above- or below-mentioned embodiments, the infectious disease is Leptospirosis. In some embodiments of each or any of the above- or below-mentioned embodiments, the infectious disease is Listeriosis (Listeria). In some embodiments of each or any of the above- or below-mentioned embodiments, the infectious disease is Lyme Disease. In some embodiments of each or any of the above- or below-mentioned embodiments, the infectious disease is Lymphogranuloma venereum infection (LGV). In some embodiments of each or any of the above- or below-mentioned embodiments, the infectious disease is Malaria. In some embodiments of each or any of the above- or below-mentioned embodiments, the infectious disease is Measles. In some embodiments of each or any of the above- or below-mentioned embodiments, the infectious disease is Melioidosis. In some embodiments of each or any of the above- or below-mentioned embodiments, the infectious disease is Meningitis (Viral). In some embodiments of each or any of the above- or below-mentioned embodiments, the infectious disease is Meningococcal Disease (Meningitis (Bacterial)). In some embodiments of each or any of the above- or below-mentioned embodiments, the infectious disease is Middle East Respiratory Syndrome Coronavirus (MERS-COV). In some embodiments of each or any of the above- or below-mentioned embodiments, the infectious disease is Mumps. In some embodiments of each or any of the above- or below-mentioned embodiments, the infectious disease is Norovirus. In some embodiments of each or any of the above- or below-mentioned embodiments, the infectious disease is Pediculosis. In some embodiments of each or any of the above- or below-mentioned embodiments, the infectious disease is Pelvic Inflammatory Disease (PID). In some embodiments of each or any of the above- or below-mentioned embodiments, the infectious disease is Pertussis (Whooping Cough). In some embodiments of each or any of the above- or below-mentioned embodiments, the infectious disease is Plague (Bubonic. In some embodiments of each or any of the above- or below-mentioned embodiments, the infectious disease is Septicemic. In some embodiments of each or any of the above- or below-mentioned embodiments, the infectious disease is Pneumonic). In some embodiments of each or any of the above- or below-mentioned embodiments, the infectious disease is Pneumococcal Disease (Pneumonia). In some embodiments of each or any of the above- or below-mentioned embodiments, the infectious disease is Poliomyelitis (Polio). In some embodiments of each or any of the above- or below-mentioned embodiments, the infectious disease is Powassan. In some embodiments of each or any of the above- or below-mentioned embodiments, the infectious disease is Psittacosis. In some embodiments of each or any of the above- or below-mentioned embodiments, the infectious disease is Pthiriasis. In some embodiments of each or any of the above- or below-mentioned embodiments, the infectious disease is Pustular Rash diseases (Small pox. In some embodiments of each or any of the above- or below-mentioned embodiments, the infectious disease is monkeypox. In some embodiments of each or any of the above- or below-mentioned embodiments, the infectious disease is cowpox). In some embodiments of each or any of the above- or below-mentioned embodiments, the infectious disease is Q-Fever. In some embodiments of each or any of the above- or below-mentioned embodiments, the infectious disease is Rabies. In some embodiments of each or any of the above- or below-mentioned embodiments, the infectious disease is Rickettsiosis (Rocky Mountain Spotted Fever). In some embodiments of each or any of the above- or below-mentioned embodiments, the infectious disease is Rubella (German Measles). In some embodiments of each or any of the above- or below-mentioned embodiments, the infectious disease is Salmonellosis gastroenteritis (Salmonella). In some embodiments of each or any of the above- or below-mentioned embodiments, the infectious disease is Scabies. In some embodiments of each or any of the above- or below-mentioned embodiments, the infectious disease is Scombroid. In some embodiments of each or any of the above- or below-mentioned embodiments, the infectious disease is Sepsis. In some embodiments of each or any of the above- or below-mentioned embodiments, the infectious disease is Severe Acute Respiratory Syndrome (SARS). In some embodiments of each or any of the above- or below-mentioned embodiments, the infectious disease is Shigellosis gastroenteritis (Shigella). In some embodiments of each or any of the above- or below-mentioned embodiments, the infectious disease is Smallpox. In some embodiments of each or any of the above- or below-mentioned embodiments, the infectious disease is Staphyloccal Infection Methicillin-resistant (MRSA). In some embodiments of each or any of the above- or below-mentioned embodiments, the infectious disease is Staphylococcal Food Poisoning Enterotoxin B Poisoning (Staph Food Poisoning). In some embodiments of each or any of the above- or below-mentioned embodiments, the infectious disease is Saphylococcal Infection Vancomycin Intermediate (VISA). In some embodiments of each or any of the above- or below-mentioned embodiments, the infectious disease is Staphylococcal Infection Vancomycin Resistant (VRSA). In some embodiments of each or any of the above- or below-mentioned embodiments, the infectious disease is Streptococcal Disease Group A (invasive) (Strep A (invasive). In some embodiments of each or any of the above- or below-mentioned embodiments, the infectious disease is Streptococcal Disease. In some embodiments of each or any of the above- or below-mentioned embodiments, the infectious disease is Group B (Strep-B). In some embodiments of each or any of the above- or below-mentioned embodiments, the infectious disease is Streptococcal Toxic-Shock Syndrome STSS Toxic Shock. In some embodiments of each or any of the above- or below-mentioned embodiments, the infectious disease is primary. In some embodiments of each or any of the above- or below-mentioned embodiments, the infectious disease is secondary. In some embodiments of each or any of the above- or below-mentioned embodiments, the infectious disease is early latent. In some embodiments of each or any of the above- or below-mentioned embodiments, the infectious disease is late latent. In some embodiments, the infectious disease is congenital. In some embodiments of each or any of the above- or below-mentioned embodiments, the infectious disease is Tetanus Infection. In some embodiments of each or any of the above- or below-mentioned embodiments, the infectious disease is Trichomoniasis. In some embodiments of each or any of the above- or below-mentioned embodiments, the infectious disease is Trichonosis Infection. In some embodiments of each or any of the above- or below-mentioned embodiments, the infectious disease is Tuberculosis (TB). In some embodiments of each or any of the above- or below-mentioned embodiments, the infectious disease is Tuberculosis Latent (LTBI). In some embodiments of each or any of the above- or below-mentioned embodiments, the infectious disease is Tularemia. In some embodiments of each or any of the above- or below-mentioned embodiments, the infectious disease is Typhoid Fever Group D. In some embodiments of each or any of the above- or below-mentioned embodiments, the infectious disease is Vaginosis. In some embodiments of each or any of the above- or below-mentioned embodiments, the infectious disease is Varicella (Chickenpox), Vibrio cholerae (Cholera). In some embodiments of each or any of the above- or below-mentioned embodiments, the infectious disease is Vibriosis (Vibrio). In some embodiments of each or any of the above- or below-mentioned embodiments, the infectious disease is Ebola Virus Hemorrhagic Fever. In some embodiments of each or any of the above- or below-mentioned embodiments, the infectious disease is Lasa Virus Hemorrhagic Fever. In some embodiments of each or any of the above- or below-mentioned embodiments, the infectious disease is Marburg Virus Hemorrhagic Fever. In some embodiments of each or any of the above- or below-mentioned embodiments, the infectious disease is West Nile Virus. In some embodiments of each or any of the above- or below-mentioned embodiments, the infectious disease is Yellow Fever. In some embodiments of each or any of the above- or below-mentioned embodiments, the infectious disease is Yersenia. In some embodiments of each or any of the above- or below-mentioned embodiments, the infectious disease is and Zika Virus Infection.

In some embodiments of each or any of the above- or below-mentioned embodiments, the pathogen is a bacteria. In some embodiments of each or any of the above- or below-mentioned embodiments, the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus. In some embodiments of each or any of the above- or below-mentioned embodiments, the bacteria is a bacteria of the bacillus genus. In some embodiments of each or any of the above- or below-mentioned embodiments, the bacteria is a bacteria of the bartonella genus. In some embodiments of each or any of the above- or below-mentioned embodiments, the bacteria is a bacteria of the bordetella genus. In some embodiments of each or any of the above- or below-mentioned embodiments, the bacteria is a bacteria of the borrelia genus. In some embodiments of each or any of the above- or below-mentioned embodiments, the bacteria is a bacteria of the brucella genus. In some embodiments of each or any of the above- or below-mentioned embodiments, the bacteria is a bacteria of the campylobacter genus. In some embodiments of each or any of the above- or below-mentioned embodiments, the bacteria is a bacteria of the chlamydia genus. In some embodiments of each or any of the above- or below-mentioned embodiments, the bacteria is a bacteria of the chlamydophila genus. In some embodiments of each or any of the above- or below-mentioned embodiments, the bacteria is a bacteria of the clostridium genus. In some embodiments of each or any of the above- or below-mentioned embodiments, the bacteria is a bacteria of the corynebacterium genus. In some embodiments of each or any of the above- or below-mentioned embodiments, the bacteria is a bacteria of the enterococcus genus. In some embodiments of each or any of the above- or below-mentioned embodiments, the bacteria is a bacteria of the escherichia genus. In some embodiments of each or any of the above- or below-mentioned embodiments, the bacteria is a bacteria of the francisella genus. In some embodiments of each or any of the above- or below-mentioned embodiments, the bacteria is a bacteria of the haemophilus genus. In some embodiments of each or any of the above- or below-mentioned embodiments, the bacteria is a bacteria of the helicobacter genus. In some embodiments of each or any of the above- or below-mentioned embodiments, the bacteria is a bacteria of the legionella genus. In some embodiments of each or any of the above- or below-mentioned embodiments, the bacteria is a bacteria of the leptospira genus. In some embodiments of each or any of the above- or below-mentioned embodiments, the bacteria is a bacteria of the listeria genus. In some embodiments of each or any of the above- or below-mentioned embodiments, the bacteria is a bacteria of the mycobacterium genus. In some embodiments of each or any of the above- or below-mentioned embodiments, the bacteria is a bacteria of the mycoplasma genus. In some embodiments of each or any of the above- or below-mentioned embodiments, the bacteria is a bacteria of the neisseria genus. In some embodiments of each or any of the above- or below-mentioned embodiments, the bacteria is a bacteria of the pseudomonas genus. In some embodiments of each or any of the above- or below-mentioned embodiments, the bacteria is a bacteria of the rickettsia genus. In some embodiments of each or any of the above- or below-mentioned embodiments, the bacteria is a bacteria of the salmonella genus. In some embodiments of each or any of the above- or below-mentioned embodiments, the bacteria is a bacteria of the shigella genus. In some embodiments of each or any of the above- or below-mentioned embodiments, the bacteria is a bacteria of the staphylococcus genus. In some embodiments of each or any of the above- or below-mentioned embodiments, the bacteria is a bacteria of the streptococcus genus. In some embodiments of each or any of the above- or below-mentioned embodiments, the bacteria is a bacteria of the treponema genus. In some embodiments of each or any of the above- or below-mentioned embodiments, the bacteria is a bacteria of the ureaplasma genus. In some embodiments of each or any of the above- or below-mentioned embodiments, the bacteria is a bacteria of the vibrio genus. In some embodiments of each or any of the above- or below-mentioned embodiments, the bacteria is a bacteria of the yersinia genus.

In some embodiments of each or any of the above- or below-mentioned embodiments, the pathogen is a parasite. In some embodiments of each or any of the above- or below-mentioned embodiments, the parasite is a protozoa, helminth, or ectoparasite. In some embodiments of each or any of the above- or below-mentioned embodiments, the protozoa is an entamoeba, giardia, leishmania, balantidium, plasmodium, or cryptosporidium. In some embodiments of each or any of the above- or below-mentioned embodiments, the helminth is a trematode, cestode, acanthocephalan, or round worm. In some embodiments of each or any of the above- or below-mentioned embodiments, the ectoparasite is an arthropod.

In some embodiments of each or any of the above- or below-mentioned embodiments, the pathogen is a virus. In some embodiments of each or any of the above- or below-mentioned embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments of each or any of the above- or below-mentioned embodiments, the virus is a virus of the adenoviridae family. In some embodiments of each or any of the above- or below-mentioned embodiments, the virus is a virus of the arenaviridae family. In some embodiments of each or any of the above- or below-mentioned embodiments, the virus is a virus of the astroviridae family. In some embodiments of each or any of the above- or below-mentioned embodiments, the virus is a virus of the bunyaviridae family. In some embodiments of each or any of the above- or below-mentioned embodiments, the virus is a virus of the caliciviridae family. In some embodiments of each or any of the above- or below-mentioned embodiments, the virus is a virus of the coronaviridae family. In some embodiments of each or any of the above- or below-mentioned embodiments, the virus is a virus of the filoviridae family. In some embodiments of each or any of the above- or below-mentioned embodiments, the virus is a virus of the flaviviridae family. In some embodiments of each or any of the above- or below-mentioned embodiments, the virus is a virus of the hepadnaviridae family. In some embodiments of each or any of the above- or below-mentioned embodiments, the virus is a virus of the hepeviridae family. In some embodiments of each or any of the above- or below-mentioned embodiments, the virus is a virus of the orthomyxoviridae family. In some embodiments of each or any of the above- or below-mentioned embodiments, the virus is a virus of the papillomaviridae family. In some embodiments of each or any of the above- or below-mentioned embodiments, the virus is a virus of the paramyxoviridae family. In some embodiments of each or any of the above- or below-mentioned embodiments, the virus is a virus of the parvoviridae family. In some embodiments of each or any of the above- or below-mentioned embodiments, the virus is a virus of the picornaviridae family. In some embodiments of each or any of the above- or below-mentioned embodiments, the virus is a virus of the polyomaviridae family. In some embodiments of each or any of the above- or below-mentioned embodiments, the virus is a virus of the poxviridae family. In some embodiments of each or any of the above- or below-mentioned embodiments, the virus is a virus of the reoviridae family. In some embodiments of each or any of the above- or below-mentioned embodiments, the virus is a virus of the retroviridae family. In some embodiments of each or any of the above- or below-mentioned embodiments, the virus is a virus of the rhabdoviridae family. In some embodiments of each or any of the above- or below-mentioned embodiments, the virus is a virus of the togaviridae family.

In some embodiments of each or any of the above- or below-mentioned embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments of each or any of the above- or below-mentioned embodiments, the virus is an adenovirus. In some embodiments of each or any of the above- or below-mentioned embodiments, the virus is a coronavirus. In some embodiments of each or any of the above- or below-mentioned embodiments, the coronavirus virus is Covid-19 (SARS-COV-2). In some embodiments of each or any of the above- or below-mentioned embodiments, the virus is a coxsackievirus. In some embodiments of each or any of the above- or below-mentioned embodiments, the virus is a Epstein-Barr virus. In some embodiments of each or any of the above- or below-mentioned embodiments, the virus is a hepatitis A virus. In some embodiments of each or any of the above- or below-mentioned embodiments, the virus is a hepatitis B virus. In some embodiments of each or any of the above- or below-mentioned embodiments, the virus is a hepatitis C virus. In some embodiments of each or any of the above- or below-mentioned embodiments, the virus is a herpes simplex virus type 2. In some embodiments of each or any of the above- or below-mentioned embodiments, the virus is a cytomegalovirus. In some embodiments of each or any of the above- or below-mentioned embodiments, the virus is a human herpes virus type 8. In some embodiments of each or any of the above- or below-mentioned embodiments, the virus is a human immunodeficiency virus. In some embodiments of each or any of the above- or below-mentioned embodiments, the virus is an influenza virus. In some embodiments of each or any of the above- or below-mentioned embodiments, the virus is a measles virus. In some embodiments of each or any of the above- or below-mentioned embodiments, the virus is a mumps virus. In some embodiments of each or any of the above- or below-mentioned embodiments, the virus is a human papillomavirus. In some embodiments of each or any of the above- or below-mentioned embodiments, the virus is a parainfluenza virus. In some embodiments of each or any of the above- or below-mentioned embodiments, the virus is a poliovirus. In some embodiments of each or any of the above- or below-mentioned embodiments, the virus is a rabies virus. In some embodiments of each or any of the above- or below-mentioned embodiments, the virus is a respiratory syncytial virus. In some embodiments of each or any of the above- or below-mentioned embodiments, the virus is a rubella virus. In some embodiments of each or any of the above- or below-mentioned embodiments, the virus is a varicella-zoster virus.

In some embodiments of each or any of the above- or below-mentioned embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopeniaurpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

In some embodiments of each or any of the above- or below-mentioned embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as psoriasis, rheumatoid arthritis, osteoarthritis, psoriatic arthritis, Crohn's disease, asthma, allergies or inflammatory bowel disease, are characterized by chronic inflammation. Inflammatory diseases such as arthritis, related arthritic conditions (e.g., osteoarthritis, rheumatoid arthritis, and psoriatic arthritis), inflammatory bowel disease (e.g., Crohn's disease and ulcerative colitis), sepsis, psoriasis, atopic dermatitis, contact dermatitis, and chronic obstructive pulmonary disease, chronic inflammatory pulmonary diseases are also prevalent and problematic ailments.

Various delivery systems are known and can be used to administer a prophylactic or therapeutic agent (e.g., an antibody or antigen binding fragment thereof provided herein), including, but not limited to, encapsulation in liposomes, microparticles, microcapsules, recombinant cells capable of expressing the antibody or antigen binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments of each or any of the above- or below-mentioned embodiments, when administering an antibody or antigen binding fragment thereof provided herein, care must be taken to use materials to which the antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibody or antigen binding fragment thereof provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more antibodies or antigen binding fragments thereof provided herein. In another embodiment, a composition provided herein comprises one, two or more antibodies or antigen binding fragments thereof provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In some embodiments of each or any of the above- or below-mentioned embodiments, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antibody or antigen binding fragment thereof provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In some embodiments of each or any of the above- or below-mentioned embodiments, the antibody or antigen binding fragment thereof is supplied as a dry sterilized lyophilized powder or water free concentrate in a hermetically sealed container and can be reconstituted, e.g., with water or saline to the appropriate concentration for administration to a subject. The lyophilized antibody or antigen binding fragment thereof can be stored at between 2 and 8° C. in its original container and the antibody or antigen binding fragment thereof can be administered within 12 hours, such as within 6 hours, within 5 hours, within 3 hours, or within 1 hour after being reconstituted. In some embodiments of each or any of the above- or below-mentioned embodiments, an antibody or antigen binding fragment thereof provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody or antigen binding fragment thereof provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In some embodiments of each or any of the above- or below-mentioned embodiments, the route of administration for a dose of an antibody or antigen binding fragment thereof provided herein to a patient is intranasal, intramuscular, intravenous, subcutaneous, or a combination thereof, but other routes described herein are also acceptable. Each dose may or may not be administered by an identical route of administration. In some embodiments of each or any of the above- or below-mentioned embodiments, an antibody or antigen binding fragment thereof provided herein may be administered via multiple routes of administration simultaneously or subsequently to other doses of the same or a different antibody or antigen binding fragment thereof provided herein.

In certain embodiments, the antibody or antigen binding fragment thereof provided herein are administered prophylactically or therapeutically to a subject. The antibody or antigen binding fragment thereof provided herein can be prophylactically or therapeutically administered to a subject so as to prevent, lessen or ameliorate a disease or symptom thereof.

### 5.8 Gene Therapy

In a specific embodiment, nucleic acids comprising sequences encoding antibodies or functional derivatives thereof, are administered to a subject for use in a method provided herein, for example, to prevent, manage, treat and/or ameliorate a disease, disorder or condition, by way of gene therapy. Such therapy encompasses that performed by the administration to a subject of an expressed or expressible nucleic acid. In some embodiments of each or any of the above- or below-mentioned embodiments, the nucleic acids produce their encoded antibody, and the antibody mediates a prophylactic or therapeutic effect. Any of the methods for recombinant gene expression (or gene therapy) available in the art can be used.

In some embodiments, the nucleic acid for the gene therapy encodes a binding molecule as disclosed herein or a fragment thereof. In some embodiments, the nucleic acid for the gene therapy encodes a binding molecule comprising a region derived from a CH1 region of an antibody heavy chain and/or a region derived from a CL region of an antibody light chain, wherein the region derived from the CH1 region and/or the region derived from the CL region comprises one or more antigen binding loop(s). In some embodiments, the nucleic acid for the gene therapy encodes a first polypeptide comprising a heavy chain variable region (VH) and a region derived from a CH1 region of an antibody heavy chain, wherein the region derived from the CH1 region comprises one or more antigen binding loop(s). In some embodiments, the nucleic acid for the gene therapy encodes a second polypeptide comprising a light chain variable region (VL) and a region derived from a CL region of an antibody light chain, wherein the region derived from the CL region comprises one or more antigen binding loop(s). In some embodiments, one or more nucleic acid molecule(s) for the gene therapy encodes (i) a first polypeptide comprising a heavy chain variable region (VH) and a region derived from a CH1 region of an antibody heavy chain, and (ii) a second polypeptide comprising a light chain variable region (VL) and a region derived from a CL region of an antibody light chain, wherein the region derived from the CH1 region and/or the region derived from the CL region comprises one or more antigen binding loop(s).

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215. Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990).

In a specific embodiment, a composition comprises nucleic acids encoding an antibody provided herein, the nucleic acids being part of an expression vector that expresses the antibody or chimeric proteins or heavy or light chains thereof in a suitable host. In particular, such nucleic acids have promoters, such as heterologous promoters, operably linked to the antibody coding region, the promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In some embodiments of each or any of the above- or below-mentioned embodiments, the expressed antibody molecule is a single chain antibody; alternatively, the nucleic acid sequences include sequences encoding both the heavy and light chains, or fragments thereof, of the antibody.

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68: 143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an antibody as provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.9 Diagnostic Assays and Methods

Labeled antibodies and derivatives and analogs thereof, which immunospecifically bind to an antigen provided herein can be used for diagnostic purposes to detect, diagnose, or monitor a disease or disorder.

Antibodies provided herein can be used to assay an antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin. One aspect provided herein is the detection and diagnosis of a disease or disorder in a human.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a disease or disorder is carried out by repeating the method for diagnosing the disease or disorder, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.10 Kits

Also provided herein are kits comprising an antibody provided herein, or a composition (e.g., a pharmaceutical composition) provided herein, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to a specific antigen. In specific embodiments, provided herein are panels of antibodies having different association rate constants different dissociation rate constants, different affinities for an antigen, and/or different specificities for an antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EMBODIMENTS

This invention provides the following non-limiting embodiments.

In one set of embodiments (embodiment set A), provided are:
A1. (Embodiment 1) A binding molecule comprising:
   (i) a first polypeptide comprising a heavy chain variable region (VH) and a region derived from a CH1 region of an antibody heavy chain, and
   (ii) a second polypeptide comprising a light chain variable region (VL) and a region derived from a CL region of an antibody light chain,
   wherein the region derived from the CH1 region and/or the region derived from the CL region comprises one or more antigen binding loop(s).
A2. (Embodiment 2) The binding molecule of embodiment A1, wherein the one or more antigen binding loop(s) in the region derived from the CH1 region are at the AB, BC, CD, DE, EF, and/or FG loop regions of the CH1 region.
A3. (Embodiment 3) The binding molecule of embodiment A1 or A2, wherein the one or more antigen binding loop(s) in the region derived from the CL region are at the AB, BC, CD, DE, EF, and/or FG loop regions of the CL region.
A4. (Embodiment 4) The binding molecule of any one of embodiments A1-A3, wherein the region derived from the CH1 region comprises one or two antigen binding loop(s).
A5. (Embodiment 5) The binding molecule of any one of embodiments A1-A4, wherein the region derived from the CL region comprises one or two antigen binding loop(s).
A6. (Embodiment 6) The binding molecule of any one of embodiments A1-A5, wherein the region derived from the CH1 region comprises one antigen binding loop at the CD loop region of the CH1 region.
A7. (Embodiment 7) The binding molecule of any one of embodiments A1-A6, wherein the region derived from the CH1 region comprises one antigen binding loop at the DE loop region of the CH1 region.
A8. (Embodiment 8) The binding molecule of any one of embodiments A1-A7, wherein the region derived from the CH1 region comprises one antigen binding loop at the CD loop region of the CH1 region and one antigen binding loop at the DE loop region of the CH1 region.
A9. (Embodiment 9) The binding molecule of any one of embodiments A1-A8, wherein the region derived from the CL region comprises one antigen binding loop at the CD loop region of the CL region.
A10. (Embodiment 10) The binding molecule of any one of embodiments A1-A9, wherein the region derived from the CL region comprises one antigen binding loop at the DE loop region of the CL region.
A11. (Embodiment 11) The binding molecule of any one of embodiments A1-A10, wherein the region derived from the CL region comprises one antigen binding loop at the CD loop region of the CL region and one antigen binding loop at the DE loop region of the CL region.
A12. (Embodiment 12) The binding molecule of any one of embodiments A1-A6 or A9, wherein the region derived from the CH1 region comprises one antigen binding loop at the CD loop region of the CH1 region; and the region derived from the CL region comprises one antigen binding loop at the CD loop region of the CL region.
A13. (Embodiment 13) The binding molecule of any one of embodiments A1-A6 or A10, wherein the region derived from the CH1 region comprises one antigen binding loop at the CD loop region of the CH1 region; and the region derived from the CL region comprises one antigen binding loop at the DE loop region of the CL region.
A14. (Embodiment 14) The binding molecule of any one of embodiments A1-A6 or A9-A13, wherein the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region.
A15. (Embodiment 15) The binding molecule of any one of embodiments A1-A5, A7 or A9, wherein the region derived from the CH1 region comprises one antigen binding loop at the DE loop region of the CH1 region; and the region derived from the CL region comprises one antigen binding loop at the CD loop region of the CL region.
A16. (Embodiment 16) The binding molecule of any one of embodiments A1-A5, A7 or A10, wherein the region derived from the CH1 region comprises one antigen binding loop at the DE loop region of the CH1 region; and the region derived from the CL region comprises one antigen binding loop at the DE loop region of the CL region.
A17. (Embodiment 17) The binding molecule of any one of embodiments A1-A5, A7, A9-A11, A15 or A16, wherein the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region.
A18. (Embodiment 18) The binding molecule of any one of embodiments A1-A9, A12 or A15, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region.
A19. (Embodiment 19) The binding molecule of any one of embodiments A1-A8, A10, A13, or A16, wherein the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region.
A20. (Embodiment 20) The binding molecule of any one of embodiments A1-A19, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.
A21. (Embodiment 21) The binding molecule of any one of embodiments A1-A20, wherein the region derived from the CH1 region is a region derived from a human IgG1 CH1 region comprising an amino acid sequence of SEQ ID NO: 1, and wherein the region derived from the CH1 region comprises an amino acid sequence having at least 70%, 75%, 80%, 85%, 90% or 95% identity to SEQ ID NO:1.
A22. (Embodiment 22) The binding molecule of any one of embodiments A1-A21, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
A23. (Embodiment 23) The binding molecule of embodiment A21 or A22, wherein the antigen binding loop at the CD loop region of the CH1 region replaces the amino acid residues TSG of the CD loop of the human IgG1 CH1 region.
A24. (Embodiment 24) The binding molecule of embodiment A21 or A22, wherein the antigen binding loop at the DE loop region of the CH1 region replaces the amino acid residues QSS of the DE loop of the human IgG1 CH1 region.
A25. (Embodiment 25) The binding molecule of embodiment A21 or A22, wherein the antigen binding loop at the CD loop region of the CL region replaces the amino acid residues SGNS of the CD loop of the human CL kappa region.
A26. (Embodiment 26) The binding molecule of embodiment A21 or A22, wherein the antigen binding loop at the DE loop region of the CL region replaces the amino acid residues SKD of the DE loop of the human CL kappa region.
A27. (Embodiment 27) The binding molecule of any one of embodiments A1-A26, wherein each of the one or more antigen binding loop(s) comprises 7 to 15 amino acid residues.
A28. (Embodiment 28) The binding molecule of any one of embodiments A1-A27, wherein the VH region and the VL region bind to an first antigen; and the region derived from the CH1 region and/or the region derived from the CL region bind to a second antigen.
A29. (Embodiment 29) The binding molecule of embodiment A28, wherein the first antigen and the second antigen are the same antigen.
A30. (Embodiment 30) The binding molecule of embodiment A28, wherein the first antigen and the second antigen are two different antigens.
A31. (Embodiment 31) A nucleic acid encoding the binding molecule of any one of embodiments A1-A30.
A32. (Embodiment 32) A vector comprising the nucleic acid of embodiment A31.

In another set of embodiments (embodiment set B), provided are:
B1. (Embodiment 1) A constant region library (CRL) comprising a population of binding molecules, wherein each of the binding molecules comprises:
   (i) a first polypeptide comprising a heavy chain variable region (VH) and a region derived from a CH1 region of an antibody heavy chain, and
   (ii) a second polypeptide comprising a light chain variable region (VL) and a region derived from a CL region of an antibody light chain,
   wherein the population of the binding molecules comprise diverse amino acid sequences in the region derived from the CH1 region and/or the region derived from the CL region.
B2. (Embodiment 2) The CRL of embodiment B1, wherein the diverse amino acid sequences in the region derived from the CH1 region are at the AB, BC, CD, DE, EF, and/or FG loop regions of the CH1 region.
B3. (Embodiment 3) The CRL of embodiment B1 or B2, wherein the diverse amino acid sequences in the region derived from the CL region are at the AB, BC, CD, DE, EF, and/or FG loop regions of the CL region.
B4. (Embodiment 4) The CRL of any one of embodiments B1-B3, wherein the population of the binding molecules comprise diverse amino acid sequences in one or two loop region(s) in the region derived from the CH1 region.
B5. (Embodiment 5) The CRL of any one of embodiments B1-B4, wherein the population of the binding molecules comprise diverse amino acid sequences in one or two loop region(s) in the region derived from the CL region.
B6. (Embodiment 6) The CRL of any one of embodiments B1-B5, wherein the population of the binding molecules comprise diverse amino acid sequences at the CD loop region of the CH1 region.
B7. (Embodiment 7) The CRL of any one of embodiments B1-B6, wherein the population of the binding molecules comprise diverse amino acid sequences at the DE loop region of the CH1 region.
B8. (Embodiment 8) The CRL of any one of embodiments B1-B7, wherein the population of the binding molecules comprise diverse amino acid sequences at the CD loop region and the DE loop region of the CH1 region.
B9. (Embodiment 9) The CRL of any one of embodiments B1-B8, wherein the population of the binding molecules comprise diverse amino acid sequences at the CD loop region of the CL region.
B10. (Embodiment 10) The CRL of any one of embodiments B1-B9, wherein the population of the binding molecules comprise diverse amino acid sequences at the DE loop region of the CL region.
B11. (Embodiment 11) The CRL of any one of embodiments B1-B10, wherein the population of the binding molecules comprise diverse amino acid sequences at the CD loop region and the DE loop region of the CL region.
B12. (Embodiment 12) The CRL of any one of embodiments B1-B6 or B9, wherein the population of the binding molecules comprise diverse amino acid sequences at the CD loop region of the CH1 region; and the population of the binding molecules comprise diverse amino acid sequences at the CD loop region of the CL region.
B13. (Embodiment 13) The CRL of any one of embodiments B1-B6 or B10, wherein the population of the binding molecules comprise diverse amino acid sequences at the CD loop region of the CH1 region; and the population of the binding molecules comprise diverse amino acid sequences at the DE loop region of the CL region.
B14. (Embodiment 14) The CRL of any one of embodiments B1-B6 or B9-B13, wherein the population of the binding molecules comprise diverse amino acid sequences at the CD loop region of the CH1 region; and the population of the binding molecules comprise diverse amino acid sequences at the CD loop region and the DE loop region of the CL region.
B15. (Embodiment 15) The CRL of any one of embodiments B1-B5, B7 or B9, wherein the population of the binding molecules comprise diverse amino acid sequences at the DE loop region of the CH1 region; and the population of the binding molecules comprise diverse amino acid sequences at the CD loop region of the CL region.
B16. (Embodiment 16) The CRL of any one of embodiments B1-B5, B7 or B10, wherein the population of the binding molecules comprise diverse amino acid sequences at the DE loop region of the CH1 region; and the population of the binding molecules comprise diverse amino acid sequences at the DE loop region of the CL region.
B17. (Embodiment 17) The CRL of any one of embodiments B1-B5, B7, B9-B11, B15 or B16, wherein the population of the binding molecules comprise diverse amino acid sequences at the DE loop region of the CH1 region; and the population of the binding molecules comprise diverse amino acid sequences at the CD loop region and the DE loop region of the CL region.
B18. (Embodiment 18) The CRL of any one of embodiments B1-B9, B12 or B15, wherein the population of the binding molecules comprise diverse amino acid sequences at the CD loop region of the CH1 region and the DE loop region of the CH1 region; and the population of the binding molecules comprise diverse amino acid sequences at the CD loop region of the CL region.
B19. (Embodiment 19) The CRL of any one of embodiments B1-B8, B10, B13, or B16, wherein the population of the binding molecules comprise diverse amino acid sequences at the CD loop region and the DE loop region of the CH1 region; and the population of the binding molecules comprise diverse amino acid sequences at the DE loop region of the CL region.
B20. (Embodiment 20) The CRL of any one of embodiments B1-B19, wherein the population of the binding molecules comprise diverse amino acid sequences at the CD loop region and the DE loop region of the CH1 region; and the population of the binding molecules comprise diverse amino acid sequences at the CD loop region and the DE loop region of the CL region.
B21. (Embodiment 21) The CRL of any one of embodiments B1-B20, wherein the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.
B22. (Embodiment 22) The CRL of any one of embodiments B1-B21, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
B23. (Embodiment 23) The CRL of embodiment B21 or B22, wherein the amino acid residues TSG of the CD loop of the human IgG1 CH1 region are replaced with diverse amino acid sequences in the binding molecules in the CRL.
B24. (Embodiment 24) The CRL of embodiment B21 or B22, wherein the amino acid residues QSS of the DE loop of the human IgG1 CH1 region are replaced with diverse amino acid sequences in the binding molecules in the CRL.
B25. (Embodiment 25) The CRL of embodiment B21 or B22, wherein the amino acid residues SGNS of the CD loop of the human CL kappa region are replaced with diverse amino acid sequences in the binding molecules in the CRL.
B26. (Embodiment 26) The CRL of embodiment B21 or B22, wherein the amino acid residues SKD of the DE loop of the human CL kappa region are replaced with diverse amino acid sequences in the binding molecules in the CRL.
B27. (Embodiment 27) The CRL of any one of embodiments B1 to B26, wherein the diverse amino acid sequences comprise 7 to 15 amino acid residues.
B28. (Embodiment 28) A constant region library (CRL) comprising a population of molecules each comprising a region derived from a CH1 region and/or a region derived from a CL region of an antibody, wherein the population of the molecules comprise diverse amino acid sequences in the region derived from the CH1 region and/or the region derived from the CL region.
B29. (Embodiment 29) The CRL of embodiment B28, wherein the diverse amino acid sequences in the region derived from the CH1 region are at the AB, BC, CD, DE, EF, and/or FG loop regions of the CH1 region.
B30. (Embodiment 30) The CRL of embodiment B28, wherein the diverse amino acid sequences in the region derived from the CL region are at the AB, BC, CD, DE, EF, and/or FG loop regions of the CL region.
B31. (Embodiment 31) The CRL of embodiment B28, wherein the population of the molecules comprise diverse amino acid sequences in one or two loop region(s) in the region derived from the CH1 region.
B32. (Embodiment 32) The CRL of embodiment B28, wherein the population of the molecules comprise diverse amino acid sequences in one or two loop region(s) in the region derived from the CL region.
B33. (Embodiment 33) The CRL of embodiment B28, wherein the population of the molecules comprise diverse amino acid sequences at the CD loop region of the CH1 region.
B34. (Embodiment 34) The CRL of embodiment B28, wherein the population of the molecules comprise diverse amino acid sequences at the DE loop region of the CH1 region.
B35. (Embodiment 35) The CRL of embodiment B28, wherein the population of the molecules comprise diverse amino acid sequences at the CD loop region and the DE loop region of the CH1 region.
B36. (Embodiment 36) The CRL of embodiment B28, wherein the population of the molecules comprise diverse amino acid sequences at the CD loop region of the CL region.
B37. (Embodiment 37) The CRL of embodiment B28, wherein the population of the molecules comprise diverse amino acid sequences at the DE loop region of the CL region.
B38. (Embodiment 38) The CRL of embodiment B28, wherein the population of the molecules comprise diverse amino acid sequences at the CD loop region and the DE loop region of the CL region.
B39. (Embodiment 39) The CRL of embodiment B28, wherein the population of the molecules comprise diverse amino acid sequences at the CD loop region of the CH1 region; and the population of the molecules comprise diverse amino acid sequences at the CD loop region of the CL region.
B40. (Embodiment 40) The CRL of embodiment B28, wherein the population of the molecules comprise diverse amino acid sequences at the CD loop region of the CH1 region; and the population of the molecules comprise diverse amino acid sequences at the DE loop region of the CL region.
B41. (Embodiment 41) The CRL of embodiment B28, wherein the population of the molecules comprise diverse amino acid sequences at the CD loop region of the CH1 region; and the population of the molecules comprise diverse amino acid sequences at the CD loop region and the DE loop region of the CL region.
B42. (Embodiment 42) The CRL of embodiment B28, wherein the population of the molecules comprise diverse amino acid sequences at the DE loop region of the CH1 region; and the population of the molecules comprise diverse amino acid sequences at the CD loop region of the CL region.
B43. (Embodiment 43) The CRL of embodiment B28, wherein the population of the molecules comprise diverse amino acid sequences at the DE loop region of the CH1 region; and the population of the molecules comprise diverse amino acid sequences at the DE loop region of the CL region.
B44. (Embodiment 44) The CRL of embodiment B28, wherein the population of the molecules comprise diverse amino acid sequences at the DE loop region of the CH1 region; and the population of the molecules comprise diverse amino acid sequences at the CD loop region and the DE loop region of the CL region.
B45. (Embodiment 45) The CRL of embodiment B28, wherein the population of the molecules comprise diverse amino acid sequences at the CD loop region of the CH1 region and the DE loop region of the CH1 region; and the population of the molecules comprise diverse amino acid sequences at the CD loop region of the CL region.
B46. (Embodiment 46) The CRL of embodiment B28, wherein the population of the molecules comprise diverse amino acid sequences at the CD loop region and the DE loop region of the CH1 region; and the population of the molecules comprise diverse amino acid sequences at the DE loop region of the CL region.
B47. (Embodiment 47) The CRL of embodiment B28, wherein the population of the molecules comprise diverse amino acid sequences at the CD loop region and the DE loop region of the CH1 region; and the population of the molecules comprise diverse amino acid sequences at the CD loop region and the DE loop region of the CL region.
B48. (Embodiment 48) The CRL of any one of embodiments B28-B47, wherein the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.
B49. (Embodiment 49) The CRL of any one of embodiments B28-B47, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
B50. (Embodiment 50) The CRL of embodiment B48 or B49, wherein the amino acid residues TSG of the CD loop of the human IgG1 CH1 region are replaced with diverse amino acid sequences in the molecules in the CRL.
B51. (Embodiment 51) The CRL of embodiment B48 or B49, wherein the amino acid residues QSS of the DE loop of the human IgG1 CH1 region are replaced with diverse amino acid sequences in the molecules in the CRL.
B52. (Embodiment 52) The CRL of embodiment B48 or B49, wherein the amino acid residues SGNS of the CD loop of the human CL kappa region are replaced with diverse amino acid sequences in the molecules in the CRL.
B53. (Embodiment 53) The CRL of embodiment B48 or B49, wherein the amino acid residues SKD of the DE loop of the human CL kappa region are replaced with diverse amino acid sequences in the molecules in the CRL.
B54. (Embodiment 54) The CRL of any one of embodiments B28-B53, wherein the diverse amino acid sequences comprise 7 to 15 amino acid residues.
B55. (Embodiment 55) The CRL of any one of embodiments B28-B54, wherein each of the molecules further comprise a VH region and a VL region.
B56. (Embodiment 56) The CRL of any one of embodiments B1-B55, wherein the binding molecules or the molecules are Fab fragments.
B57. (Embodiment 57) The CRL of any one of embodiments B1-B56, wherein the diversity of the CRL with one loop region ranges from 10⁷ to 10¹⁶.
B58. (Embodiment 58) The CRL of any one of embodiments B1-B56, wherein the diversity of the CRL with two loop regions ranges from 10¹⁸ to 10³³.
B59. (Embodiment 59) A method for identifying a binding molecule comprising a first binding domain that binds to a first antigen and a second binding domain that binds to a second antigen, comprising screening the CRL of any one of embodiments B1-B58 for identifying the binding molecule that binds to the second antigen with a higher affinity than a reference level, wherein the first binding domain comprises the VH region and the VL region of an antibody, and wherein the second binding domain comprises an antibody constant region variant.
B60. (Embodiment 60) A method of producing a binding molecule comprising a first step for performing a function of identifying an antibody constant region variant capable of binding to an antigen; and a second step of constructing the binding molecule that comprises the antibody constant region variant.
B61. (Embodiment 61) The method of embodiment B60, wherein the first step comprising screening the CRL of any one of embodiments B1-B58.
B62. (Embodiment 62) A binding molecule produced according to the method of any one of embodiments B59-B61.

In another set of embodiments (embodiment set C), provided are:
C1. (Embodiment 1) A method of making a binding molecule, comprising expressing a polynucleotide encoding a binding molecule in a host cell, wherein the binding molecule comprises:
   (i) a first polypeptide comprising a heavy chain variable region (VH) and a region derived from a CH1 region of an antibody heavy chain, and
   (ii) a second polypeptide comprising a light chain variable region (VL) and a region derived from a CL region of an antibody light chain,
   wherein the region derived from the CH1 region and/or the region derived from the CL region comprises one or more antigen binding loop(s).
C2. (Embodiment 2) The method of embodiment C1, wherein the one or more antigen binding loop(s) in the region derived from the CH1 region are at the AB, BC, CD, DE, EF, and/or FG loop regions of the CH1 region.
C3. (Embodiment 3) The method of embodiment C1 or C2, wherein the one or more antigen binding loop(s) in the region derived from the CL region are at the AB, BC, CD, DE, EF, and/or FG loop regions of the CL region.
C4. (Embodiment 4) The method of any one of embodiments C1-C₃, wherein the region derived from the CH1 region comprises one or two antigen binding loop(s).
C5. (Embodiment 5) The method of any one of embodiments C1-C₄, wherein the region derived from the CL region comprises one or two antigen binding loop(s).
C6. (Embodiment 6) The method of any one of embodiments C1-C₅, wherein the region derived from the CH1 region comprises one antigen binding loop at the CD loop region of the CH1 region.
C7. (Embodiment 7) The method of any one of embodiments C1-C₆, wherein the region derived from the CH1 region comprises one antigen binding loop at the DE loop region of the CH1 region.
C8. (Embodiment 8) The method of any one of embodiments C1-C7, wherein the region derived from the CH1 region comprises one antigen binding loop at the CD loop region of the CH1 region and one antigen binding loop at the DE loop region of the CH1 region.
C9. (Embodiment 9) The method of any one of embodiments C1-C8, wherein the region derived from the CL region comprises one antigen binding loop at the CD loop region of the CL region.
C10. (Embodiment 10) The method of any one of embodiments C1-C9, wherein the region derived from the CL region comprises one antigen binding loop at the DE loop region of the CL region.
C11. (Embodiment 11) The method of any one of embodiments C1-C10, wherein the region derived from the CL region comprises one antigen binding loop at the CD loop region of the CL region and one antigen binding loop at the DE loop region of the CL region.
C12. (Embodiment 12) The method of any one of embodiments C1-C6 or C9, wherein the region derived from the CH1 region comprises one antigen binding loop at the CD loop region of the CH1 region; and the region derived from the CL region comprises one antigen binding loop at the CD loop region of the CL region.
C13. (Embodiment 13) The method of any one of embodiments C1-C6 or C10, wherein the region derived from the CH1 region comprises one antigen binding loop at the CD loop region of the CH1 region; and the region derived from the CL region comprises one antigen binding loop at the DE loop region of the CL region.
C14. (Embodiment 14) The method of any one of embodiments C1-C6 or C9-C13, wherein the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region.
C15. (Embodiment 15) The method of any one of embodiments C1-C₅, C7 or C9, wherein the region derived from the CH1 region comprises one antigen binding loop at the DE loop region of the CH1 region; and the region derived from the CL region comprises one antigen binding loop at the CD loop region of the CL region.
C16. (Embodiment 16) The method of any one of embodiments C1-C₅, C7 or C10, wherein the region derived from the CH1 region comprises one antigen binding loop at the DE loop region of the CH1 region; and the region derived from the CL region comprises one antigen binding loop at the DE loop region of the CL region.
C17. (Embodiment 17) The method of any one of embodiments C1-C₅, C7, C9-C11, C15 or C16, wherein the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region.
C18. (Embodiment 18) The method of any one of embodiments C1-C9, C12 or C15, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region.
C19. (Embodiment 19) The method of any one of embodiments C1-C8, C10, C13, or C16, wherein the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region.
C20. (Embodiment 20) The method of any one of embodiments C1-C19, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.
C21. (Embodiment 21) The method of any one of embodiments C1-C20, wherein the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.
C22. (Embodiment 22) The method of any one of embodiments C1-C21, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
C23. (Embodiment 23) The method of embodiment C21 or C22, wherein the antigen binding loop at the CD loop region of the CH1 region replaces the amino acid residues TSG of the CD loop of the human IgG1 CH1 region.
C24. (Embodiment 24) The method of embodiment C21 or C22, wherein the antigen binding loop at the DE loop region of the CH1 region replaces the amino acid residues QSS of the DE loop of the human IgG1 CH1 region.
C25. (Embodiment 25) The method of embodiment C21 or C22, wherein the antigen binding loop at the CD loop region of the CL region replaces the amino acid residues SGNS of the CD loop of the human CL kappa region.
C26. (Embodiment 26) The method of embodiment C21 or C22, wherein the antigen binding loop at the DE loop region of the CL region replaces the amino acid residues SKD of the DE loop of the human CL kappa region.
C27. (Embodiment 27) The method of any one of embodiments C1-C26, wherein each of the one or more antigen binding loop(s) comprises 7 to 15 amino acid residues.
C28. (Embodiment 28) The method of any one of embodiments C1-C27, wherein the VH region and the VL region bind to an first antigen; and the region derived from the CH1 region and/or the region derived from the CL region bind to a second antigen.
C29. (Embodiment 29) The method of embodiment C28, wherein the first antigen and the second antigen are the same antigen.
C30. (Embodiment 30) The method of embodiment C28, wherein the first antigen and the second antigen are two different antigens.

In yet another set of embodiments (embodiment set D), provided are:
D1. (Embodiment 1) A pharmaceutical composition comprising:
   (a) a binding molecule comprising:
      (i) a first polypeptide comprising a heavy chain variable region (VH) and a region derived from a CH1 region of an antibody heavy chain, and
      (ii) a second polypeptide comprising a light chain variable region (VL) and a region derived from a CL region of an antibody light chain,
   wherein the region derived from the CH1 region and/or the region derived from the CL region comprises one or more antigen binding loop(s); and
   (b) a pharmaceutically acceptable excipient.
D2. (Embodiment 2) The pharmaceutical composition of embodiment D1, wherein the one or more antigen binding loop(s) in the region derived from the CH1 region are at the AB, BC, CD, DE, EF, and/or FG loop regions of the CH1 region.
D3. (Embodiment 3) The pharmaceutical composition of embodiment D1 or D2, wherein the one or more antigen binding loop(s) in the region derived from the CL region are at the AB, BC, CD, DE, EF, and/or FG loop regions of the CL region.
D4. (Embodiment 4) The pharmaceutical composition of any one of embodiments D1-D3, wherein the region derived from the CH1 region comprises one or two antigen binding loop(s).
D5. (Embodiment 5) The pharmaceutical composition of any one of embodiments D1-D4, wherein the region derived from the CL region comprises one or two antigen binding loop(s).
D6. (Embodiment 6) The pharmaceutical composition of any one of embodiments D1-D5, wherein the region derived from the CH1 region comprises one antigen binding loop at the CD loop region of the CH1 region.
D7. (Embodiment 7) The pharmaceutical composition of any one of embodiments D1-D6, wherein the region derived from the CH1 region comprises one antigen binding loop at the DE loop region of the CH1 region.
D8. (Embodiment 8) The pharmaceutical composition of any one of embodiments D1-D7, wherein the region derived from the CH1 region comprises one antigen binding loop at the CD loop region of the CH1 region and one antigen binding loop at the DE loop region of the CH1 region.
D9. (Embodiment 9) The pharmaceutical composition of any one of embodiments D1-D8, wherein the region derived from the CL region comprises one antigen binding loop at the CD loop region of the CL region.
D10. (Embodiment 10) The pharmaceutical composition of any one of embodiments D1-D9, wherein the region derived from the CL region comprises one antigen binding loop at the DE loop region of the CL region.
D11. (Embodiment 11) The pharmaceutical composition of any one of embodiments D1-D10, wherein the region derived from the CL region comprises one antigen binding loop at the CD loop region of the CL region and one antigen binding loop at the DE loop region of the CL region.
D12. (Embodiment 12) The pharmaceutical composition of any one of embodiments D1-D6 or D9, wherein the region derived from the CH1 region comprises one antigen binding loop at the CD loop region of the CH1 region; and the region derived from the CL region comprises one antigen binding loop at the CD loop region of the CL region.
D13. (Embodiment 13) The pharmaceutical composition of any one of embodiments D1-D6 or D10, wherein the region derived from the CH1 region comprises one antigen binding loop at the CD loop region of the CH1 region; and the region derived from the CL region comprises one antigen binding loop at the DE loop region of the CL region.
D14. (Embodiment 14) The pharmaceutical composition of any one of embodiments D1-D6 or D9-D13, wherein the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region.
D15. (Embodiment 15) The pharmaceutical composition of any one of embodiments D1-D5, D7 or D9, wherein the region derived from the CH1 region comprises one antigen binding loop at the DE loop region of the CH1 region; and the region derived from the CL region comprises one antigen binding loop at the CD loop region of the CL region.
D16. (Embodiment 16) The pharmaceutical composition of any one of embodiments D1-D5, D7 or D10, wherein the region derived from the CH1 region comprises one antigen binding loop at the DE loop region of the CH1 region; and the region derived from the CL region comprises one antigen binding loop at the DE loop region of the CL region.
D17. (Embodiment 17) The pharmaceutical composition of any one of embodiments D1-D5, D7, D9-D11, D15 or D16, wherein the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region.
D18. (Embodiment 18) The pharmaceutical composition of any one of embodiments D1-D9, D12 or D15, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region.
D19. (Embodiment 19) The pharmaceutical composition of any one of embodiments D1-D8, D10, D13, or D16, wherein the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region.
D20. (Embodiment 20) The pharmaceutical composition of any one of embodiments D1-D19, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.
D21. (Embodiment 21) The pharmaceutical composition of any one of embodiments D1-D20, wherein the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.
D22. (Embodiment 22) The pharmaceutical composition of any one of embodiments D1-D21, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
D23. (Embodiment 23) The pharmaceutical composition of embodiment D21 or D22, wherein the antigen binding loop at the CD loop region of the CH1 region replaces the amino acid residues TSG of the CD loop of the human IgG1 CH1 region.
D24. (Embodiment 24) The pharmaceutical composition of embodiment D21 or D22, wherein the antigen binding loop at the DE loop region of the CH1 region replaces the amino acid residues QSS of the DE loop of the human IgG1 CH1 region.
D25. (Embodiment 25) The pharmaceutical composition of embodiment D21 or D22, wherein the antigen binding loop at the CD loop region of the CL region replaces the amino acid residues SGNS of the CD loop of the human CL kappa region.
D26. (Embodiment 26) The pharmaceutical composition of embodiment D21 or D22, wherein the antigen binding loop at the DE loop region of the CL region replaces the amino acid residues SKD of the DE loop of the human CL kappa region.
D27. (Embodiment 27) The pharmaceutical composition of any one of embodiments D1-D26, wherein each of the one or more antigen binding loop(s) comprises 7 to 15 amino acid residues.
D28. (Embodiment 28) The pharmaceutical composition of any one of embodiments D1-D27, wherein the VH region and the VL region bind to an first antigen; and the region derived from the CH1 region and/or the region derived from the CL region bind to a second antigen.
D29. (Embodiment 29) The pharmaceutical composition of embodiment D28, wherein the first antigen and the second antigen are the same antigen.
D30. (Embodiment 30) The pharmaceutical composition of embodiment D28, wherein the first antigen and the second antigen are two different antigens.

In yet another set of embodiments (embodiment set E), provided are:
E1. (Embodiment 1) A method of treating a disease or disorder in a subject, comprising administering to the subject a binding molecule, wherein the binding molecule comprises:
   (i) a first polypeptide comprising a heavy chain variable region (VH) and a region derived from a CH1 region of an antibody heavy chain, and
   (ii) a second polypeptide comprising a light chain variable region (VL) and a region derived from a CL region of an antibody light chain,
   wherein the region derived from the CH1 region and/or the region derived from the CL region comprises one or more antigen binding loop(s).
E2. (Embodiment 2) The method of embodiment E1, wherein the one or more antigen binding loop(s) in the region derived from the CH1 region are at the AB, BC, CD, DE, EF, and/or FG loop regions of the CH1 region.
E3. (Embodiment 3) The method of embodiment E1 or E2, wherein the one or more antigen binding loop(s) in the region derived from the CL region are at the AB, BC, CD, DE, EF, and/or FG loop regions of the CL region.
E4. (Embodiment 4) The method of any one of embodiments E1-E3, wherein the region derived from the CH1 region comprises one or two antigen binding loop(s).
E5. (Embodiment 5) The method of any one of embodiments E1-E4, wherein the region derived from the CL region comprises one or two antigen binding loop(s).
E6. (Embodiment 6) The method of any one of embodiments E1-E5, wherein the region derived from the CH1 region comprises one antigen binding loop at the CD loop region of the CH1 region.
E7. (Embodiment 7) The method of any one of embodiments E1-E6, wherein the region derived from the CH1 region comprises one antigen binding loop at the DE loop region of the CH1 region.
E8. (Embodiment 8) The method of any one of embodiments E1-E7, wherein the region derived from the CH1 region comprises one antigen binding loop at the CD loop region of the CH1 region and one antigen binding loop at the DE loop region of the CH1 region.
E9 (Embodiment 9) The method of any one of embodiments E1-E8, wherein the region derived from the CL region comprises one antigen binding loop at the CD loop region of the CL region.
E10. (Embodiment 10) The method of any one of embodiments E1-E9, wherein the region derived from the CL region comprises one antigen binding loop at the DE loop region of the CL region.
E11. (Embodiment 11) The method of any one of embodiments E1-E10, wherein the region derived from the CL region comprises one antigen binding loop at the CD loop region of the CL region and one antigen binding loop at the DE loop region of the CL region.
E12. (Embodiment 12) The method of any one of embodiments E1-E6 or E9, wherein the region derived from the CH1 region comprises one antigen binding loop at the CD loop region of the CH1 region; and the region derived from the CL region comprises one antigen binding loop at the CD loop region of the CL region.
E13. (Embodiment 13) The method of any one of embodiments E1-E6 or E10, wherein the region derived from the CH1 region comprises one antigen binding loop at the CD loop region of the CH1 region; and the region derived from the CL region comprises one antigen binding loop at the DE loop region of the CL region.
E14. (Embodiment 14) The method of any one of embodiments E1-E6 or E9-E13, wherein the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region.
E15. (Embodiment 15) The method of any one of embodiments E1-E5, E7 or E9, wherein the region derived from the CH1 region comprises one antigen binding loop at the DE loop region of the CH1 region; and the region derived from the CL region comprises one antigen binding loop at the CD loop region of the CL region.
E16. (Embodiment 16) The method of any one of embodiments E1-E5, E7 or E10, wherein the region derived from the CH1 region comprises one antigen binding loop at the DE loop region of the CH1 region; and the region derived from the CL region comprises one antigen binding loop at the DE loop region of the CL region.
E17. (Embodiment 17) The method of any one of embodiments E1-E5, E7, E9-E11, E15 or E16, wherein the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region.
E18. (Embodiment 18) The method of any one of embodiments E1-E9, E12 or E15, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region.
E19. (Embodiment 19) The method of any one of embodiments E1-E8, E10, E13, or E16, wherein the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region.
E20. (Embodiment 20) The method of any one of embodiments E1-E19, wherein the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.
E21. (Embodiment 21) The method of any one of embodiments E1-E20, wherein the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1.
E22. (Embodiment 22) The method of any one of embodiments E1-E21, wherein the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.
E23. (Embodiment 23) The method of embodiment E21 or E22, wherein the antigen binding loop at the CD loop region of the CH1 region replaces the amino acid residues TSG of the CD loop of the human IgG1 CH1 region.
E24. (Embodiment 24) The method of embodiment E21 or E22, wherein the antigen binding loop at the DE loop region of the CH1 region replaces the amino acid residues QSS of the DE loop of the human IgG1 CH1 region.
E25. (Embodiment 25) The method of embodiment E21 or E22, wherein the antigen binding loop at the CD loop region of the CL region replaces the amino acid residues SGNS of the CD loop of the human CL kappa region.
E26. (Embodiment 26) The method of embodiment E21 or E22, wherein the antigen binding loop at the DE loop region of the CL region replaces the amino acid residues SKD of the DE loop of the human CL kappa region.
E27. (Embodiment 27) The method of any one of embodiments E1-E26, wherein each of the one or more antigen binding loop(s) comprises 7 to 15 amino acid residues.
E28. (Embodiment 28) The method of any one of embodiments E1-E27, wherein the VH region and the VL region bind to an first antigen; and the region derived from the CH1 region and/or the region derived from the CL region bind to a second antigen.
E29. (Embodiment 29) The method of embodiment E28, wherein the first antigen and the second antigen are the same antigen.
E30. (Embodiment 30) The method of embodiment E28, wherein the first antigen and the second antigen are two different antigens.
E31. (Embodiment 31) The method of any one of embodiments E1-E30, wherein the disease or disorder is associated with the first antigen and/or the second antigen.

### 7. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1—Constructing Fab Constant Region Libraries

The construction of Fab Constant Region Libraries (CRLs) was based on an anti-XO1B1 Fab fused to the M13 filamentous bacteriophage coat protein, pIX. The variable region of the anti-XO1B1 parent Fab binds to XO1B1, a monoclonal antibody directed against human thrombin. Designed libraries are constructed in solvent-accessible regions of the CD and DE loops of the CH1 and/or CL constant regions of the anti-XO1B1 parent Fab (shown in FIG. 1B) to enable binding to a second antigen. CRLs were generated using synthetic DNA fragments (obtained from DNA synthesis vendors) encompassing the described library variants. Using standard molecular biology techniques, these DNA library fragments were subcloned in a dual gene E. coli phage display vector, where the Fab heavy chain is in frame with the amino terminus of the pIX phage coat protein and the light chain is expressed under control of a second promoter.

Fab CRLs were constructed using different loop combinations and lengths to replace portions of the CD and DE loops of the CH1 and/or CL constant regions of the parent Fab. For instance, a first Generation (G1) Fab CRL was constructed using a diversified 7 or 9 amino acid binding loop to replace either (A) three amino acids (TSG₁₆₄₋₁₆₆, EU numbering) within CH1 CD loop, (B) three amino acids (SKD₁₆₈₋₁₇₀, EU numbering) within CL kappa DE loop, or (C) both loops; a second Generation (G2) Fab CRL was constructed by using the same 7 and 9 amino acid binding loops to replace either (A) three amino acids (QSS₁₇₅₋₁₇₇, EU numbering) within CH1 DE loop, (B) four amino acids (SGNS₁₅₆₋₁₅₉, EU numbering) within CL kappa CD loop, or (C) both loops; the fifth Generation (G5) Fab CRL was constructed by using a diversified 15 amino acid binding loop to replace either (A) S₁₆₅ within CH1 CD loop, (B) K₁₆₉ within CL kappa DE loop, or (C) both loops.

The varied compositions of Fab CRLs' binding loops are shown in FIG. 1C. The amino acid cysteine was excluded from the CRLs due to its propensity to form unwanted disulfide bonds. Methionine was excluded due to its propensity to undergo oxidation. The steric and hydropathic properties of phenylalanine are largely recapitulated by the side-chains of tyrosine and tryptophan, so phenylalanine was excluded from the CRLs. Similarly, the steric and hydropathic properties of isoleucine are largely recapitulated by the side-chains of valine and leucine, so isoleucine was excluded from the CRLs. The first and last residues of the diversified sequences were referred to as anchors. For the 15 amino acid CRLs, alanine and glycine were selected as anchors at positions −1, −2, 16 and 17. Glycine offers maximum backbone flexibility in the event that presentation of the diversified loop to its target required backbone torsions that are disfavored by the other 19 canonical amino acids. Alanine provides a minimal degree of backbone constraint that may stabilize appropriate presentation of the diversified region in the event that such presentation tolerates reduced torsional flexibility. For the 7 and 9 amino acid CRLs, anchor residues were selected according to their small, hydrophilic side-chains. For amino acids with duplicated side-chain functionality (aspartate and glutamate, asparagine and glutamine), the larger amino acid was excluded from the anchor positions. Arginine was included at the anchor positions due to the ability of its guanidinium side-chain functionality to form stabilizing interactions with the backbone and side-chain atoms of neighboring residues. The set of anchor residues thus comprised alanine, aspartate, glycine, asparagine, arginine, serine and threonine. Within the diversified loops of the 9 amino acid CRL, the bulky hydrophobic residues tryptophan, valine, and leucine were allowed only at alternating positions to prevent presentation of extensive hydrophobic surfaces that might bind targets non-specifically. Likewise these residues were allowed only as a cluster at positions 3, 4, and 5 in the 7 amino acid CRL. The 15 amino acid CRL was constrained only by exclusion of cysteine, methionine, phenylalanine, and isoleucine. As a result of the varied compositions of Fab CRLs' binding loops, the diversity of a Fab CRL with a single loop ranges from 10⁷ to 10¹⁶; the diversity of a Fab CRL with double loops ranges from 10¹⁸ to 10³³.

### Example 2—Selecting Fab Constant Region Binders to Anti-polyhistidine Monoclonal Antibody

In order to validate phage-displayed CRLs for evolving completely new molecular recognition to a target of interest, selections were first performed against a commercial anti-polyhistidine monoclonal antibody since the binding motif (polyhistidine) is known and is contained within the designed libraries. In total three panning pools were used in this selection. All of the three pools were based on CRL Generation 1 (G1) as described in Example 1. Specifically, the Fab CRL used in panning pool 3 (P3) was constructed by using diversified 7 and 9 amino acid binding loops to replace three amino acids (SKD₁₆₈₋₁₇₀, EU numbering) within CL kappa DE loop; the Fab CRL used in panning pool 4 (P4) was constructed by using diversified 7 and 9 amino acid binding loops to replace three amino acids (TSG₁₆₄₋₁₆₆, EU numbering) within CH1 CD loop; the Fab CRL used in panning pool 5 (P5) was constructed by using 9 amino acid binding loops to replace three amino acids (SKD₁₆₈₋₁₇₀, EU numbering) within CL kappa DE loop and three amino acids (TSG₁₆₄₋₁₆₆, EU numbering) within CH1 CD loop. The concentrations of anti-polyhistidine monoclonal antibody used in round 1, 3, 4, 5, and 6 of panning were 100 nM, 50 nM, 10 nM, 10 nM, and 10 nM, respectively. Round 2 panning was against XO1B1 to remove any CRL Fabs that do not maintain CDR-mediated target binding.

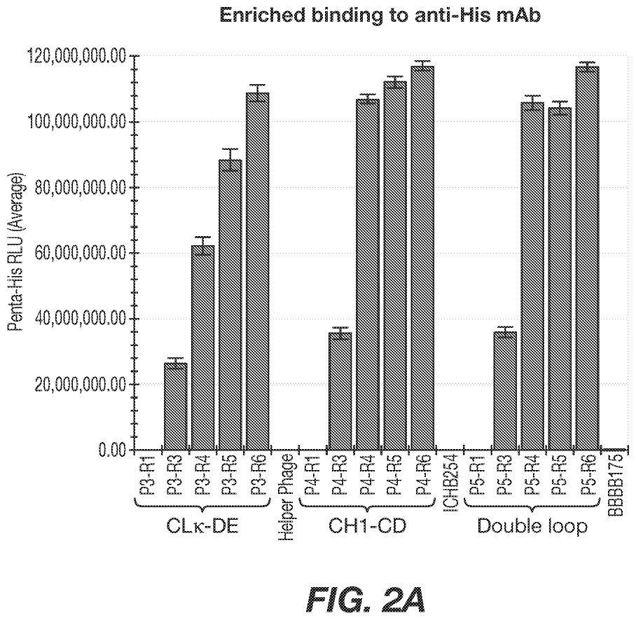

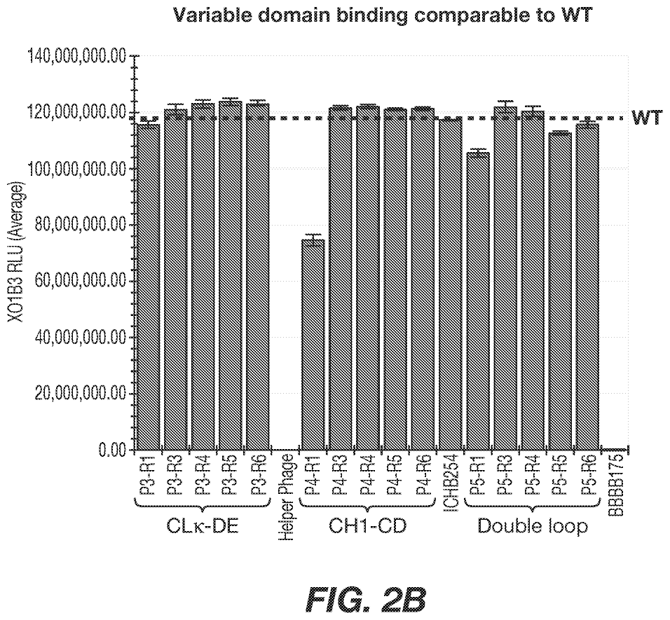

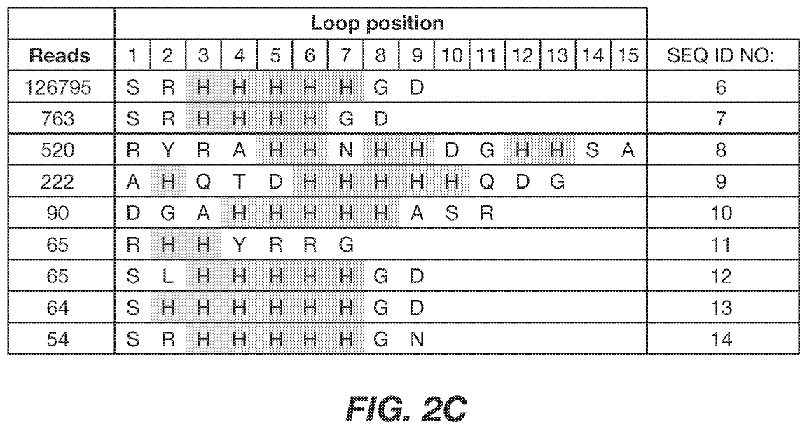

After each round of panning (except round 2), an aliquot of the output phage from each round of the three pools was analyzed by polyclonal phage ELISA for anti-polyhistidine monoclonal antibody binding and XO1B1 binding. As shown in FIG. 2A, the pools were gradually enriched with Fab constant region binders binding to anti-polyhistidine monoclonal antibody. As shown in FIG. 2B, the binding of the enriched pools against XO1B1 is comparable to that of the parent Fab. Furthermore, as demonstrated by the sequencing result shown in FIG. 2C, the originally diversified binding loops of the Fab CRL were enriched for histidine, confirming that Fab constant region binders to a target of interest can be selected from Fab CRLs.

### Example 3—Selecting Fab Constant Region Binders to mEphA2-Fc De Novo Fab CRLs Panning Against mEphA2-Fc

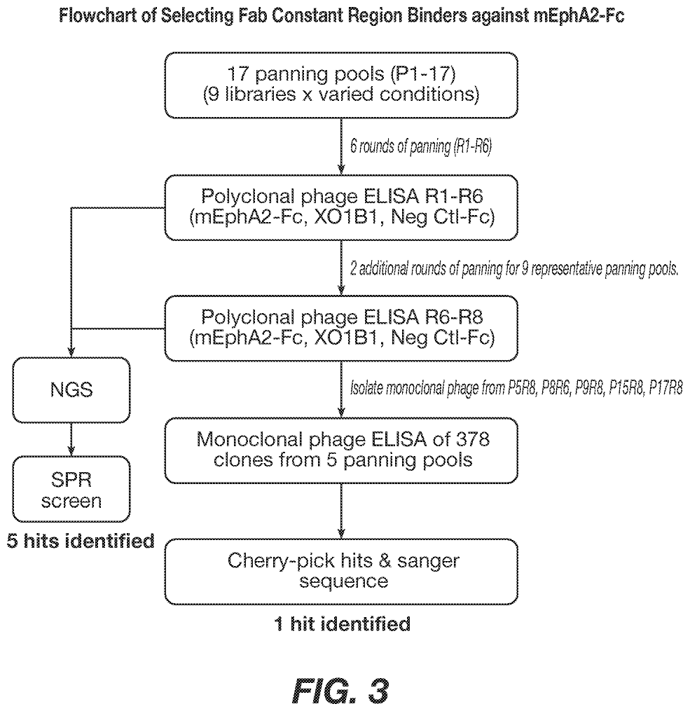

The overall process of selecting Fab constant region binders to recombinant murine EphA2-human IgG1 Fc chimera (herein referred to as mEphA2-Fc) from Fab CRLs is shown in FIG. 3. G1, G2, and G5 single and double loop Fab CRLs went through multiple rounds of panning against mEphA2-Fc under specific panning conditions. After each round of panning, the pools were analyzed by polyclonal phage ELISA for binding to mEphA2-Fc, XO1B1, and a negative control Fc fusion protein. Any pools showing enrichment for EphA2 binding were further analyzed by monoclonal phage ELISA to identify clonal mEphA2-Fc binders, which were subsequently sequenced. Next generation sequencing (NGS) was also applied to selected panning pools to identify additional potential Fab constant region binders to mEphA2-Fc. Finally, hits identified by both methods (phage ELISA and NGS) were further characterized following purification from mammalian expression.

In total seventeen panning conditions, referred to as panning pools P1-P17, were explored to identify conditions that were most productive in yielding hits against the mEphA2-Fc antigen. For general panning methods, refer to Antibody Phage Display, Methods and Protocols by Robert Aitken (ISBN 978-1-60327-302-2). In all cases, selections were carried out by binding the CRL to non-specifically biotinylated mEphA2-Fc antigen captured on neutravidin beads in the presence of a block mixture. A human IgG1 Fc competitor was used in all rounds of panning for all pools to prevent enrichment of binders to the Fc region of the mEphA2-Fc antigen. After extensive washing, the bound phage were used to infect MC1061F′ E. coli bacteria cells and amplified for subsequent panning rounds or characterization by phage ELISA. The panning conditions that were varied included (1) which library was used in the panning, (2) the amount of antigen used in each panning round, (3) the length of antigen binding time, and (4) the method for maintaining CDR-mediated binding to XO1B1. The specific panning condition of each pool during panning rounds one to six is listed in Table 2 below.

TABLE 2
Conditions for Fab CRLs Panning against mEphA2-Fc

| | | mEphA2-Fc | Overnight | XO1B1 | Library |
|---|---|---|---|---|---|
| Panning | | Concentration | Antigen Binding | (100 nM) in | Capped with |
| Pool | CRL | (nM) | in Rounds 1 and 6 | Round 2 | XO1B1 (10 nM) |
| P1 | G1 CL | 200, 100, 100, | No | Yes | No |
| | kappa- | 100, 100 | | | |
| | DE loop | | | | |
| P2 | G1 | 200, 100, 100, | No | Yes | No |
| | CH1- | 100, 100 | | | |
| | CD loop | | | | |
| P3 | G1 | 200, 100, 100, | No | Yes | No |
| | double | 100, 100 | | | |
| | loop | | | | |
| P4 | G2 CL | 200, 100, 100, | No | Yes | No |
| | kappa- | 100, 100* | | | |
| | CD loop | | | | |
| P5 | G2 | 200, 100, 100, | No | Yes | No |
| | CH1- | 100, 100* | | | |
| | DE loop | | | | |
| P6 | G2 | 200, 100, 100, | No | Yes | No |
| | double | 100, 100 | | | |
| | loop | | | | |
| P7 | G5 CL | 200, 100, 100, | No | Yes | No |
| | kappa- | 100, 100 | | | |
| | DE loop | | | | |
| P8 | G5 | 200, 100, 100, | No | Yes | No |
| | CH1- | 100, 100 | | | |
| | CD loop | | | | |
| P9 | G5 | 200, 100, 100, | No | Yes | No |
| | double | 100, 100* | | | |
| | loop | | | | |
| P10 | G1 | 200, 100, 50, 10, | No | Yes | No |
| | double | 10 | | | |
| | loop | | | | |
| P11 | G5 CL | 200, 100, 50, 10, | No | Yes | No |
| | kappa- | 10* | | | |
| | DE loop | | | | |
| P12 | G1 | 200, 100, 100, | No | Yes | Yes |
| | double | 100, 100 | | | |
| | loop | | | | |
| P13 | G5 CL | 200, 100, 100, | No | Yes | Yes |
| | kappa- | 100, 100* | | | |
| | DE loop | | | | |
| P14 | G1 | 200, 100, 100, | Yes | Yes | No |
| | double | 100, 100* | | | |
| | loop | | | | |
| P15 | G5 CL | 200, 100, 100, | Yes | Yes | No |
| | kappa- | 100, 100* | | | |
| | DE loop | | | | |
| P16 | G1 | 200, 100, 100, | Yes | No | No |
| | double | 100, 100 | | | |
| | loop | | | | |
| P17 | G5 CL | 200, 100, 100, | Yes | No | No |
| | kappa- | 100, 100* | | | |
| | DE loop | | | | |

*Two additional panning rounds were performed with overnight antigen binding

Each of the seventeen pools went through six to eight rounds of panning against mEphA2-Fc. After six to eight rounds of panning, an aliquot of the output phage from each round of the seventeen pools was analyzed by polyclonal phage ELISA for mEphA2-Fc binding relative to the anti-XO1B1 parent Fab.

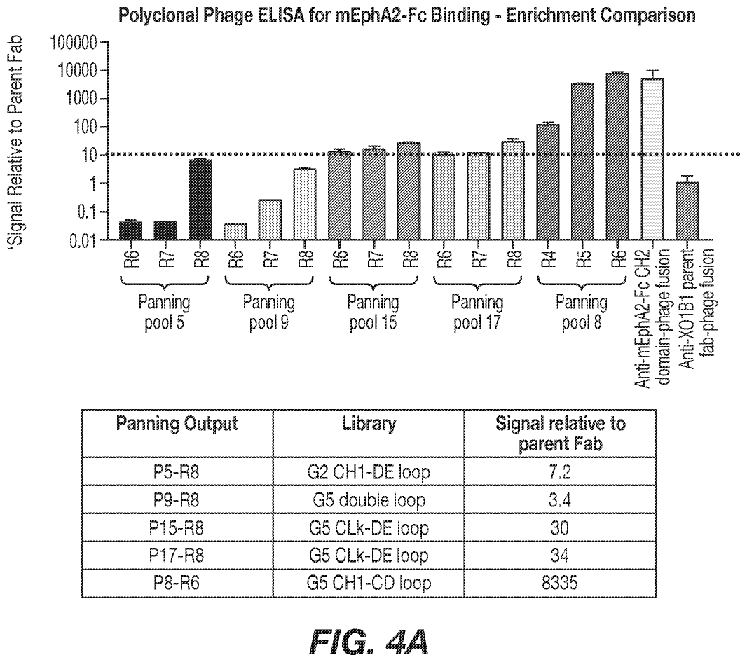

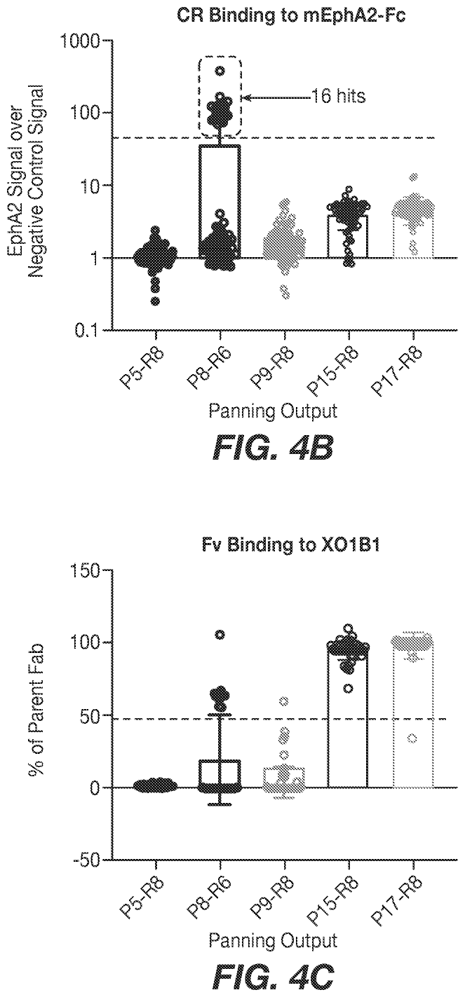

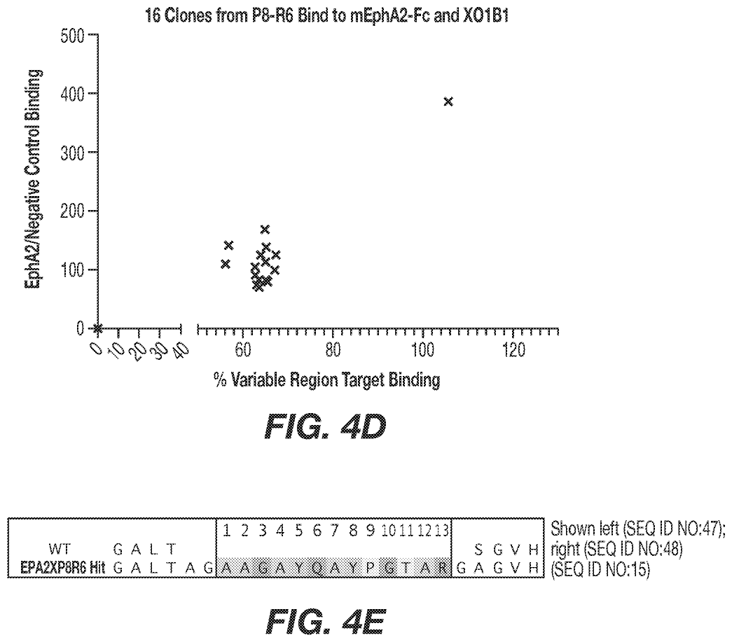

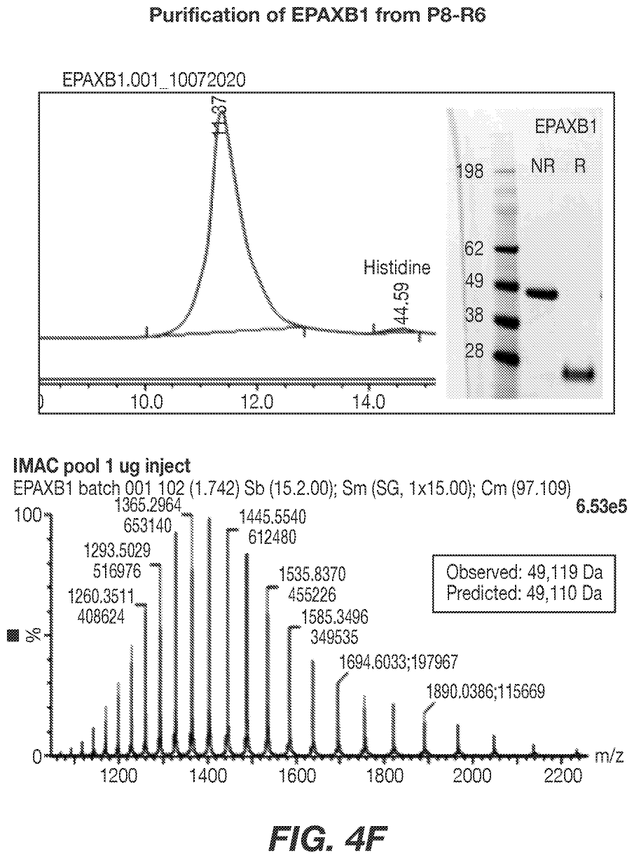

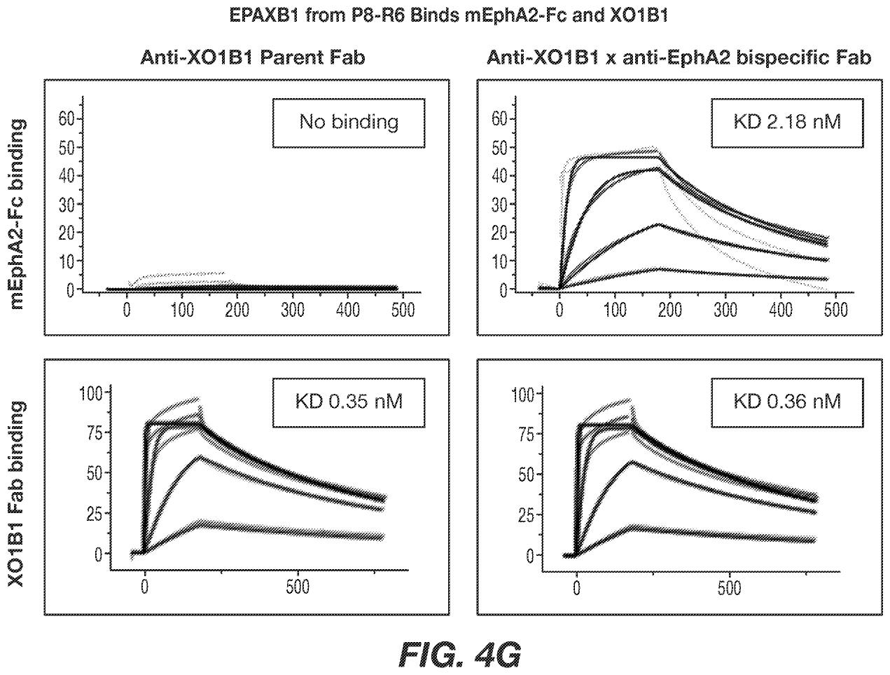

According to the result of polyclonal phage ELISA for mEphA2-Fc binding, twelve out of the seventeen pools showed no enrichment for mEphA2-Fc binding; four pools (P5-R8, P9-R8, P15-R8, P17-R8) showed weak enrichment (3.4-34-fold binding signal of phage library pool relative to the anti-XO1B1 parent Fab) for mEphA2-Fc binding; one pool (P8-R6) showed robust enrichment (8335-fold binding signal of phage library pool relative to the anti-XO1B1 parent Fab) for mEphA2-Fc binding. A summary of the results of polyclonal phage ELISA for mEphA2-Fc binding of the five enriched pools (P5, P8, P9, P15, P17) after four to eight rounds of panning is shown in FIG. 4A, which highlights that the G5 libraries with the longer 15 amino acid loops were the most productively enriched in the mEphA2-Fc panning.

To assess the binding properties of individual clones within the enriched phage pools, a total of 378 clones from the five enriched pools (P5, P8, P9, P15, P17) were further analyzed by monoclonal phage ELISA binding to mEphA2-Fc (FIG. 4B) and XO1B1 (FIG. 4C). Single clones were selected for Sanger sequencing analysis if the mEphA2-Fc binding signal over that of the negative control was no less than 60 and the XO1B1 binding signal relative to that of the parent Fab was no less than 50%. This selection criteria resulted in sixteen clones being chosen from P8, which is derived from the G5 CH1-CD loop library. P5 (G2 CH1-DE loop library) clones lost all CDR-mediated binding to XO1B1, indicating potential misfolding or truncation of clones in this panning pool. P9 (G5 double loop library) clones also had a large reduction in XO1B1 binding and only modest increases in EphA2 binding, so no clones were chosen for further sequence analysis from this pool. While clones from P15 and P17 (G5 CL kappa-DE loop library) maintained XO1B1 binding similar to the parent anti-XO1B1 Fab, the mEphA2-Fc binding signals did not meet the selection criteria, and no clones were chosen for further sequence analysis. The sixteen selected clones from P8 after six rounds of panning are shown in FIG. 4D, with a majority of clones having a similar mEphA2-Fc and XO1B1 binding profile. The sixteen selected clones were sequenced, and all of the sixteen selected clones had the same anti-mEphA2-Fc binding sequence in the CH1 CD loop, which is shown in FIG. 4E. Interestingly, the selected loop was 13 amino acids in length, representing a truncation variant derived from the designed 15 amino acid loop library.

To confirm the binding properties of the identified clone, this single Fab constant region binder to mEphA2-Fc (identified as EPAXB1) was purified as a His-tagged fusion from mammalian expression in HEK Expi293 cells. After a single-step purification by Immobilized Metal Affinity Chromatography (IMAC), a yield of 269 mg protein per liter expression volume was achieved. The protein was characterized by analytical size exclusion-high performance liquid chromatography (SE-HPLC), reducing (R) and non-reducing (NR) sodium dodecyl sulfate-polyacrylamide gel electrophoresis SDS-PAGE, and intact mass spectrometry (MS). A single peak (aside from a histidine buffer peak) was observed by SE-HPLC and high purity was observed by SDS-PAGE. The observed molecular weight (49,119 Da) of the intact purified protein was near the predicted molecular weight of 49,110 Da. The purified protein analysis is shown in FIG. 4F.

To confirm bispecificity of the purified Fab, the binding kinetics and affinity of EPAXB1 against both mEphA2-Fc and XO1B1 were analyzed using surface plasmon resonance (SPR). The binding kinetics and affinity of the anti-XO1B1 parent Fab against both mEphA2-Fc and XO1B1 were also analyzed as a reference. As shown in FIG. 4G, the parent Fab does not bind to mEphA2-Fc, while EPAXB1 binds with 2.18 nM affinity, confirming that the CRL loop identified during the panning confers novel binding in the Fab constant region. In the meantime, EPAXB1 can bind XO1B1 with a comparable affinity (KD=0.36 nM) as that of the parent Fab (KD=0.35 nM), indicating that the CDR-mediated binding is not perturbed by the addition of the CRL loop.

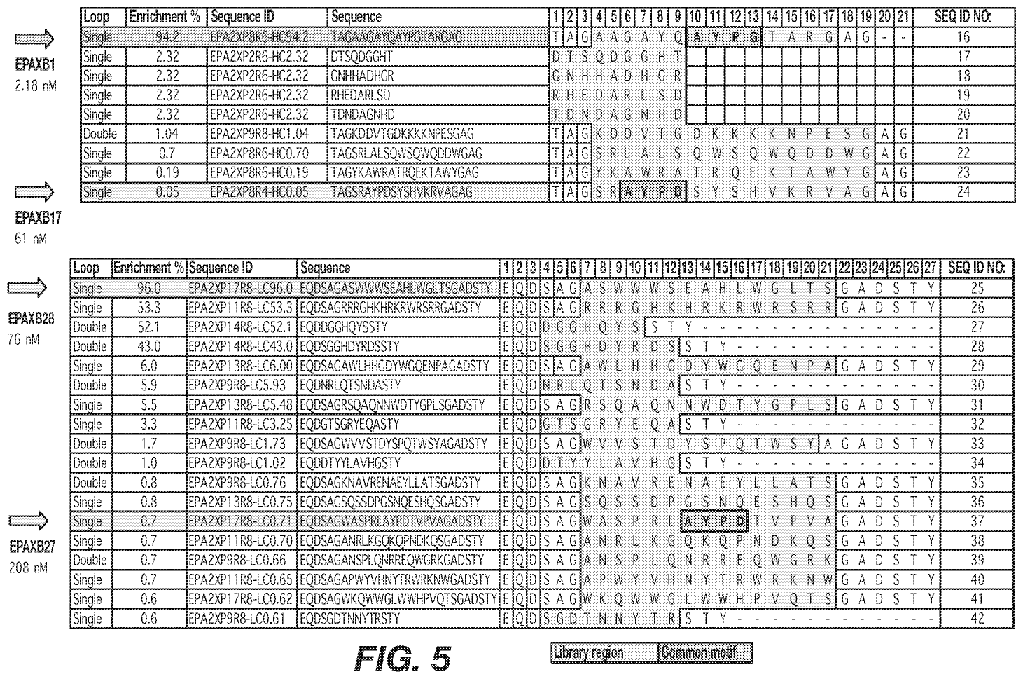

Because the number of hits identified by phage ELISA may be limited by assay sensitivity, a subset of the panning pools was also submitted for next generation sequencing (NGS) to identify additional Fab constant region binders by sequence enrichment. Based on the results from the polyclonal phage ELISA, the later panning rounds (rounds 4, 5, 6, or 8) from thirteen of the seventeen panning pools were selected for AmpliconEZ NGS analysis in the library regions. Enrichment was calculated as the ratio of the number of instances of a particular sequence over the total number of full length sequences observed multiplied by 100 percent. The anti-mEphA2 hit identified by phage ELISA was also observed by NGS, and this sequence had an enrichment of 94.2%. Enrichment above 0.5% without obvious non-specific binding motifs, such as arginine rich motifs, was used as a guide to choose sequences for further binding analysis. Accordingly, in total forty-three NGS-based hits, including two double loop variants, were cloned as His-tagged Fabs into mammalian expression vectors. These forty-three Fabs plus the single Fab constant region binder (EPAXB1) identified through phage ELISA and the parent anti-XO1B1 Fab were expressed from HEK Expi293 cells, and the supernatant was analyzed by SPR. Four out of the forty-three Fabs showed binding to mEphA2-Fc at nM range. However, one of the potential hits identified was later shown to have negligible expression levels, and was therefore not confirmed as an EphA2 binder. As a result, three new Fab constant region binders (identified as EPAXB17, EPAXB27, and EPAXB28) were identified through NGS coupled to SPR screening, which are shown in FIG. 5.

In summary, starting with seventeen different panning conditions based on loops comprising 7, 9, or 15 diversified amino acids in either the Fab CH1 and/or CL regions, in total four Fab constant region binders were identified against mEphA2-Fc. All of them were from the single loop Generation 5 (G5) Fab CRLs. The features of the four Fab constant region binders are shown in Table 3 below. An Ala-Tyr-Pro motif was present in three of the four identified sequences.

TABLE 3
Features of Fab Constant Region Binders to mEphA2-Fc

| | Panning | | Identified | | mEphA2- |
|---|---|---|---|---|---|
| Name | Pool | Library | by | Binding loop | Fc Affinity |
| EPAXB1 | P8-R6 | GS | Phage | AGAAGAYQAYPGTAR--GA | 2.18 nM |
| | | CH1:CD | ELISA and | (SEQ ID NO: 43) | |
| | | loop | NGS | | |
| EPAXB17 | P8-R4,5 | GS | NGS | AGSRAYPDSYSHVKRVAGA | 61.2 nM |
| | | CH1:CD | | (SEQ ID NO: 44) | |
| | | loop | | | |
| EPAXB28 | P17/P15- | G5 CL | NGS | AGASWWWSEAHLWGLTSGA | 76.4 nM |
| | R8 | kappa:DE | | (SEQ ID NO: 45) | |
| | | loop | | | |
| EPAXB27 | P17-R8 | G5 CL | NGS | AGWASPRLAYPDTVPVAGA | 208 nM |
| | | kappa:DE | | (SEQ ID NO: 46) | |
| | | loop | | | |

Thermal Stability of Fabs with Engineered Anti-EphA2 Binding Loops

Each of the four Fabs with engineered anti-EphA2 binding loops were analyzed by Differential Scanning Fluorimetry (DSF) to measure thermal stability. Thermal stability was determined by differential scanning fluorimetry (DSF) using a Prometheus NanoDSF instrument (Nanotemper technologies). Samples were diluted to 0.5 mg/mL in PBS pH 7.4 and loaded into a 24-well capillary from a 384 well sample plate. Duplicate runs were performed for each sample. Samples were heated from 20° C. to 95° C. at a rate of 1.0° C./minute and intrinsic tryptophan and tyrosine fluorescence was measured using an excitation wavelength of 330 nm and emission of 350 nm. The on-set temperature of aggregation using back reflection technology was also monitored. Melting temperatures and onset of aggregation were determined with Pr.Stability Analysis v1.0.2 software.

As shown in Table 4 below, each of the four Fabs with engineered anti-EphA2 binding loops have some destabilization compared to the parent Fab. The two Fabs that bind EphA2 through an engineered CD loop in the CH1 domain (i.e., EPAXB1 and EPAXB17) have only a 2° C. loss in Tm compared to the parent Fab. However, a very broad transition was observed, possibly indicating early unfolding of CH1. Due to this broad transition, T-onset is the best metric for comparison, and the bispecific Fabs have a 10-12° C. earlier onset than the parent Fab. The two Fabs that bind EphA2 through an engineered DE loop in the CLK domain (i.e., EPAXB27 and EPAXB28) have a 9° C. loss in Tm compared to the parent Fab, and a 9° C. earlier T-onset. Notably, the bispecific antibodies with the engineered loops have Tm's and T-onsets in the range of an unrelated control antibody (CNTO5825) that is known to be stable and have good biophysical properties.

TABLE 4
Thermal Stability of Fab Constant Region Binders

| | | T- | | | | | Initial |
|---|---|---|---|---|---|---|---|
| Sample ID | Description | onset | Tm1 | Tm2 | Tm3 | Tagg | Ratio |
| EPAXB1 | Anti-XO1B1 × Anti- | 61.76 | 77.26 | | | 69.14 | 0.63 |
| | EphA2 bispecific Fab | | | | | | |
| | with binding in the | | | | | | |
| | CH1:CD loop | | | | | | |
| EPAXB17 | Anti-XO1B1 × Anti- | 63.04 | 77.86 | | | 69.64 | 0.59 |
| | EphA2 bispecific Fab | | | | | | |
| | with binding in the | | | | | | |
| | CH1:CD loop | | | | | | |
| EPAXB27 | Anti-XO1B1 × Anti- | 64.57 | 70.81 | | | 67.31 | 0.69 |
| | EphA2 bispecific Fab | | | | | | |
| | with binding in the | | | | | | |
| | CLk:DE loop | | | | | | |
| EPAXB28 | Anti-XO1B1 × Anti- | 64.94 | 70.65 | | | 66.82 | 0.78 |
| | EphA2 bispecific Fab | | | | | | |
| | with binding in the | | | | | | |
| | CLk:DE loop | | | | | | |
| ICHB254 | Anti-XO1B1 Parent | 73.89 | 79.28 | | | 73.99 | 0.63 |
| | Fab, no engineered | | | | | | |
| | loops | | | | | | |
| CNTO5825 | Unrelated, stable | 64.31 | 70.43 | 80.25 | 89.96 | 82.23 | 0.78 |
| | control antibody | | | | | | |

Reformatting Fab Constant Region Binders to mEphA2-Fc

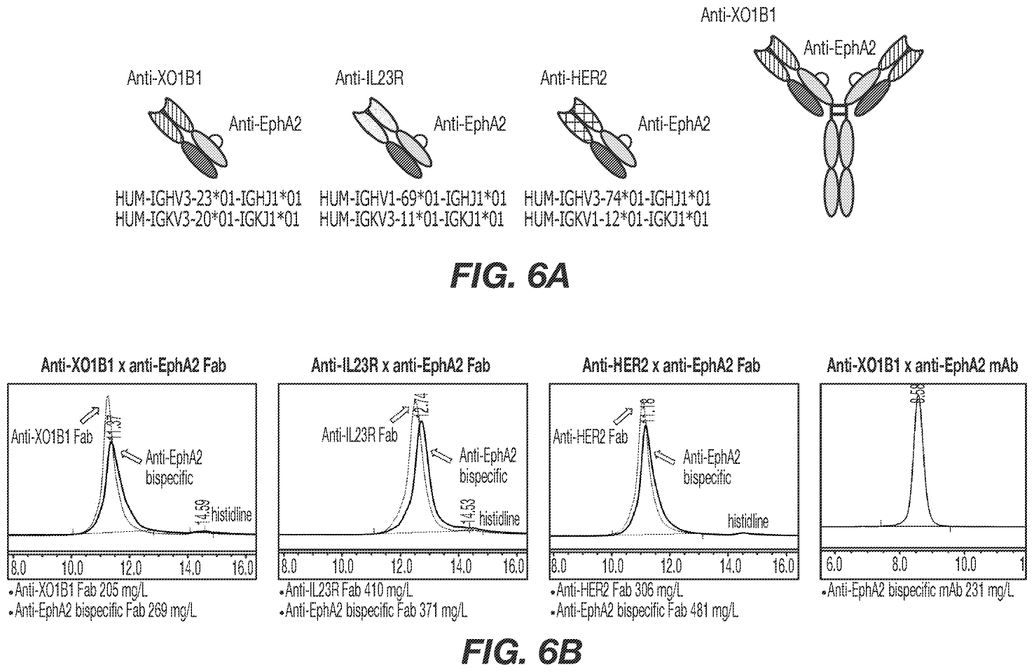

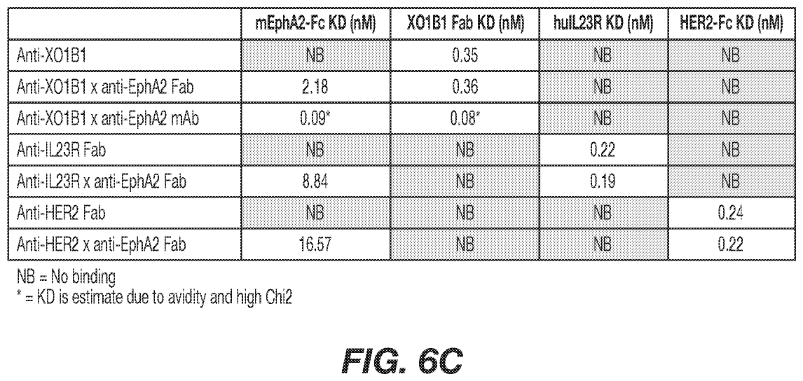

While the mEphA2-Fc binding loop in the CH1 domain of EPAXB1 was discovered in the context of the anti-XO1B1 Fab, it was paired with new variable regions (anti-human IL23R and HER2) derived from distinct human germline sequences to determine if mEphA2-Fc binding would be retained. In addition, EPAXB1 was reformatted as a monoclonal antibody (FIG. 6A). Each form was expressed in HEK 293Expi cells and purified by IMAC (for Fabs) or Protein A purification (for monoclonal antibody). Expression yields ranged from 231-481 mg of protein per liter expression for the bispecific Fabs and monoclonal antibody, and a single peak (aside from buffer peaks) was observed by SE-HPLC for each of the new formats. For the Fabs, the purification yields and SE-HPLC retention times of the bispecifics were comparable to their corresponding monospecific Fab parent (FIG. 6B).

The binding affinity of the purified monospecific and bispecific Fabs and the purified bispecific monoclonal antibody against each target (mEphA2-Fc, XO1B1, human IL23R, and HER2-Fc) were determined by Surface Plasmon Resonance (SPR). The EphA2 binding loop retains binding in all new formats, with an affinity of 2.18 nM in the anti-XO1B1 bispecific Fab, 8.84 nM in the anti-IL23R bispecific Fab, and 16.57 nM in the anti-HER2 bispecific Fab. In each of the Fabs, the variable region affinity for its cognate target is maintained in the anti-EphA2 bispecific format. In the anti-XO1B1×anti-EphA2 monoclonal antibody format, the apparent affinity of the variable region for XO1B1 is 0.08 nM and the apparent affinity of the anti-EphA2 loop for mEphA2-Fc is 0.09 nM. The apparent binding is tighter in the monoclonal antibody format compared to the Fab due to avidity (FIG. 6C).

Target Binding of Additional Bispecific mAbs

Anti-IL23R×anti-EphA2 and anti-HER2×anti-EphA2 Fabs were reformatted into standard monoclonal antibody formats and evaluated for their ability to bind their respective targets on cells.

The HER2×EphA2 and IL23×EphA2 bispecific antibodies, along with the corresponding HER2 and IL23R monospecific mAbs, were expressed in HEK 293Expi cells and purified by Protein A resin. Protein purity was assessed by SE-HPLC (size exclusion-high performance liquid chromatography).

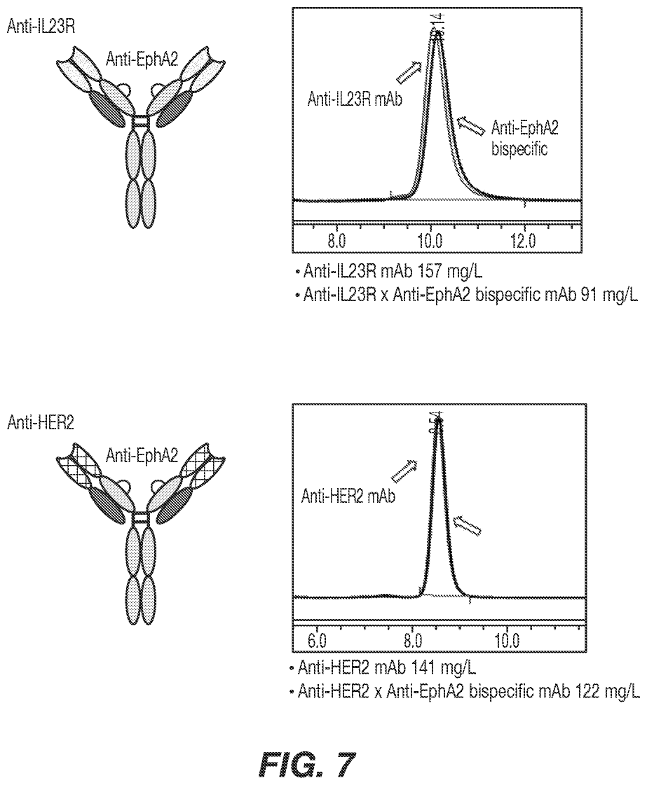

Purification yields ranged from 91-157 mg of protein per L expression, and a single peak was observed by SE-HPLC (FIG. 7). The purification yields and SE-HPLC retention times of the bispecifics were comparable to their corresponding monospecific mAb parent.

Plasmids encoding full length human EphA2 and HER2 receptors were transfected into 293F cells to make stable cell pools under hygromycin selection. The selected pools were screened by FACS using either an anti-EphA2 antibody conjugated with phycoerythrin fluorophore (R&D Systems Cat #FAB3035P) or anti-HER2 antibody (abCam Cat #11710) with detection using an anti-rat secondary antibody (Jackson Cat #112-116-143) conjugated with phycoerythrin fluorophore. All of the selected pools were isolated with their primary antibody and isotype specific Dynabeads. For EphA2, Dynabead goat anti-mouse (Thermo Cat #11033), and for HER2, Dynabead sheep anti-rat (Thermo Cat #11035) were used.

For binding studies, cells were detached from culture flasks with Accutase, washed, and resuspended in BD Stain Buffer. The cells were seeded into 96-well v-bottom plates at 150,000 cells/well and incubated 1 hour on ice with primary staining antibodies, which were serially diluted 1:2 from 400 nM. The cells were then washed and incubated with goat AF488 anti-hlgG Fcg-specific F(ab′)2 secondary detection reagent for 1 hour on ice. The cells were washed, fixed with BD Cytofix, washed again, and resuspended in 50 μl stain buffer. The cells were then analyzed on an iQue VBR Plus flow cytometer.

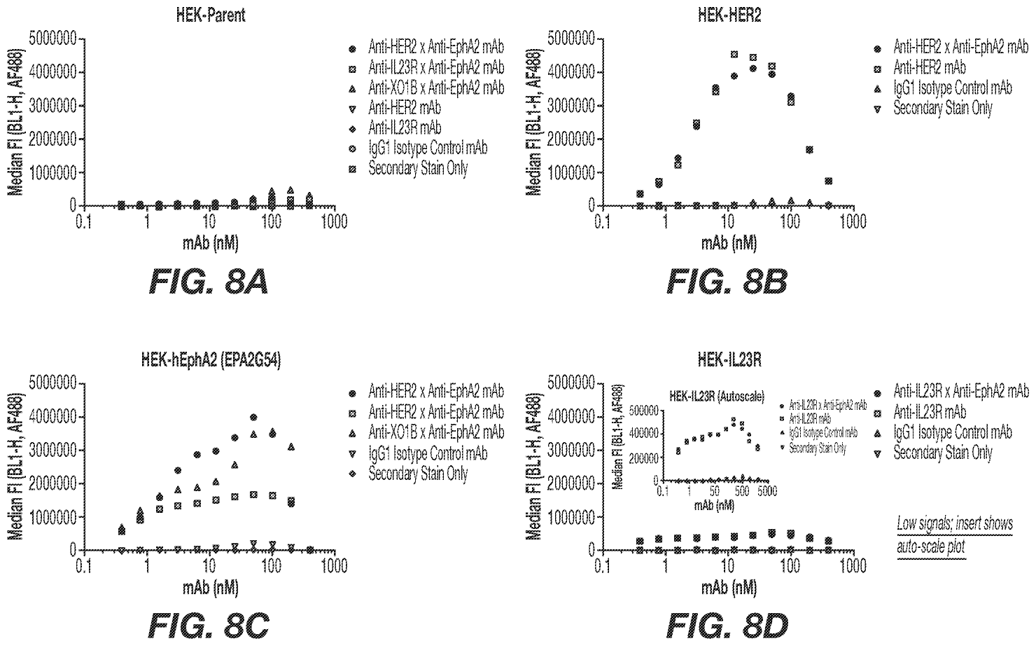

Minimal binding of the HER2×EphA2 and IL23×EphA2 bispecific antibodies or their corresponding monospecific control antibodies was observed to the untransfected HEK cells (FIG. 8A). Each of the antibodies containing the EphA2 binding loop in the antibody constant region bound to the HEK cells stably expressing human EphA2, while the isotype control did not bind appreciably (FIG. 8C). The anti-HER2×anti-EphA2 bispecific bound to the HEK-HER2 cells similarly to the parent anti-HER2 mAb (FIG. 8B), and the anti-IL23R×anti-EphA2 bispecific bound to the HEK-IL23R cells similarly to the parent anti-IL23R mAb (FIG. 8D), indicating that the presence of the EphA2 binding loop did not impact cell binding mediated by the variable region.

To further confirm specificity of the constant region binding interaction, bispecific Fabs and mAbs and their corresponding monospecific parent antibodies were also evaluated by an SPR method to determine if they had non-specific interactions with a subset of unrelated, recombinant test proteins with a range of biophysical properties. For most bispecific Fabs and mAbs containing the engineered EphA2 binding loop, no binding to the recombinant test proteins was observed. In one case, a non-specific interaction was observed, but this was attributable to the parent antibody, and not the engineered constant region loop (data not shown).

Simultaneous Target Binding of Bispecific Fabs and mAbs

Bispecific Fabs and mAbs were evaluated for their ability to simultaneously engage their targets through variable region-mediated binding and constant region loop binding.

Dual target engagement was assessed by biolayer interferometry (BLI) using an Octet RED® 384 System (ForteBio, Sartorius). Biotinlylated antigens were loaded onto streptavidin coated biosensors to reach a 1 nm response unit (RU) shift. Monospecific or bispecific mAbs (1 μM) or Fabs (400 nM) were associated for 3 mins. Secondary antigens were then associated for 3 mins. All samples were diluted into running buffer of DPBS, 0.1% BSA, and 0.02% Surfactant P20.

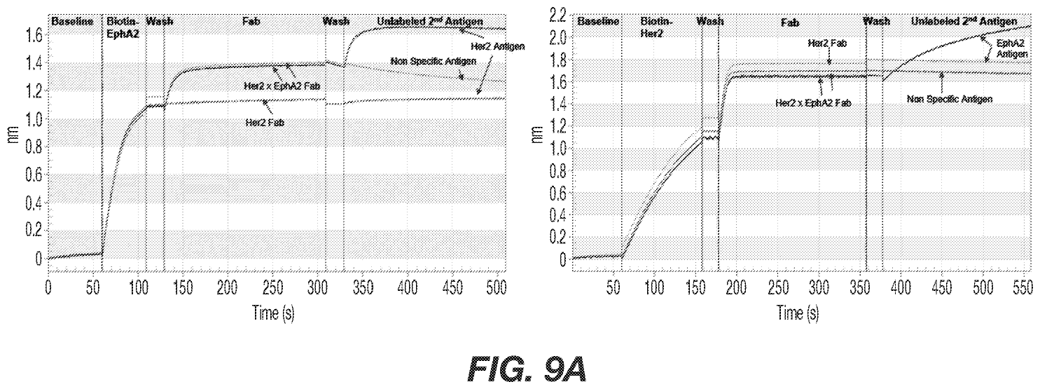

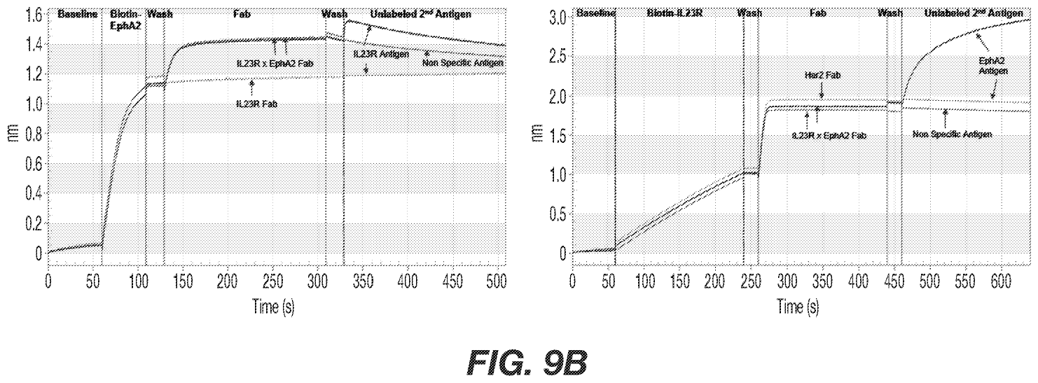

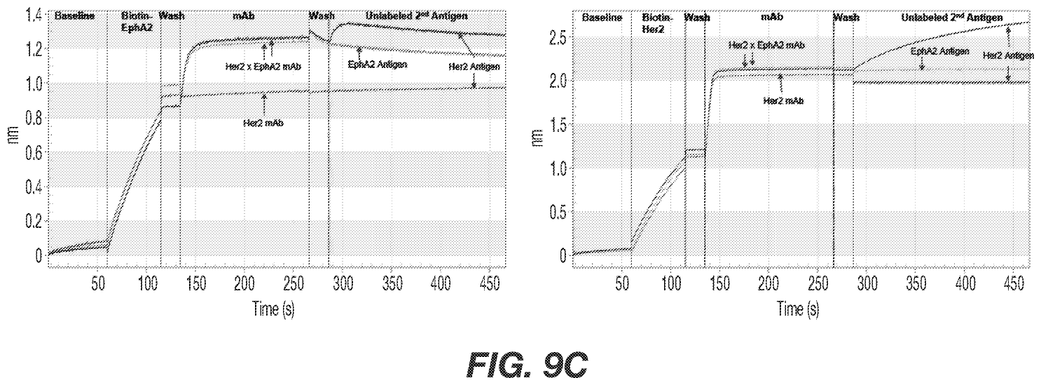

The anti-HER2×anti-EphA2 and anti-IL23R×anti-EphA2 bispecific Fabs were shown to simultaneously bind to their respective targets by biolayer interferometry (BLI) (FIGS. 9A and 9B, respectively). The dual target engagement was successful independent of which antigen was captured first on the biosensor. The anti-HER2×anti-EphA2 bispecific in mAb format was also able to simultaneously engage both targets (FIG. 9C).

1. A binding molecule comprising a region derived from a CH1 region of an antibody heavy chain and/or a region derived from a CL region of an antibody light chain, wherein the region derived from the CH1 region and/or the region derived from the CL region comprises one or more antigen binding loop(s).

2. A binding molecule comprising:
(i) a first polypeptide comprising a heavy chain variable region (VH) and a region derived from a CH1 region of an antibody heavy chain; and
(ii) a second polypeptide comprising a light chain variable region (VL) and a region derived from a CL region of an antibody light chain,
wherein the region derived from the CH1 region and/or the region derived from the CL region comprises one or more antigen binding loop(s).

3. The binding molecule of claim 1, wherein:
(i) the one or more antigen binding loop(s) in the region derived from the CH1 region are at the AB, BC, CD, DE, EF, and/or FG loop regions of the CH1 region; and/or
(ii) the one or more antigen binding loop(s) in the region derived from the CL region are at the AB, BC, CD, DE, EF, and/or FG loop regions of the CL region.

4. The binding molecule of claim 1, wherein:
(i) the region derived from the CH1 region comprises one or two antigen binding loop(s); and/or
(ii) the region derived from the CL region comprises one or two antigen binding loop(s).

5. The binding molecule of claim 1, wherein:
(i) the region derived from the CH1 region comprises one antigen binding loop at the CD loop region of the CH1 region; and/or
(ii) the region derived from the CH1 region comprises one antigen binding loop at the DE loop region of the CH1 region.

6. The binding molecule of claim 1, wherein the region derived from the CH1 region comprises one antigen binding loop at the CD loop region of the CH1 region and one antigen binding loop at the DE loop region of the CH1 region.

7. The binding molecule of claim 1, wherein the region derived from the CL region comprises:
(i) one antigen binding loop at the CD loop region of the CL region;
(ii) one antigen binding loop at the DE loop region of the CL region; or
(iii) one antigen binding loop at the CD loop region of the CL region and one antigen binding loop at the DE loop region of the CL region.

8. The binding molecule of claim 2, wherein:
(i) the region derived from the CH1 region comprises one antigen binding loop at the CD loop region of the CH1 region; and the region derived from the CL region comprises one antigen binding loop at the CD loop region of the CL region;
(ii) the region derived from the CH1 region comprises one antigen binding loop at the CD loop region of the CH1 region; and the region derived from the CL region comprises one antigen binding loop at the DE loop region of the CL region;
(iii) the region derived from the CH1 region comprises one antigen binding loop at the CD loop region of the CH1 region; and the region derived from the CL region comprises one antigen binding loop at the CD loop region of the CL region and one antigen binding loop at the DE loop region of the CL region;
(iv) the region derived from the CH1 region comprises one antigen binding loop at the DE loop region of the CH1 region; and the region derived from the CL region comprises one antigen binding loop at the CD loop region of the CL region;
(v) the region derived from the CH1 region comprises one antigen binding loop at the DE loop region of the CH1 region; and the region derived from the CL region comprises one antigen binding loop at the DE loop region of the CL region;
(vi) the region derived from the CH1 region comprises one antigen binding loop at the DE loop region of the CH1 region; and the region derived from the CL region comprises one antigen binding loop at the CD loop region of the CL region and one antigen binding loop at the DE loop region of the CL region;
(vii) the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region;
(viii) the region derived from the CH1 region comprises one antigen binding loop at the CD loop region of the CH1 region and one antigen binding loop at the DE loop region of the CH1 region; and the region derived from the CL region comprises one antigen binding loop at the DE loop region of the CL region; or
(ix) the region derived from the CH1 region comprises one antigen binding loop at the CD loop region of the CH1 region and one antigen binding loop at the DE loop region of the CH1 region; and the region derived from the CL region comprises one antigen binding loop at the CD loop region of the CL region and one antigen binding loop at the DE loop region of the CL region.

9. The binding molecule of claim 1, wherein:
(i) the region derived from the CH1 region is a region derived from a human IgG1 CH1 region comprising an amino acid sequence of SEQ ID NO:1, and wherein the region derived from the CH1 region comprises an amino acid sequence having at least 70%, 75%, 80%, 85%, 90% or 95% identity to SEQ ID NO:1; and/or
(ii) the region derived from the CL region is a region derived from a human CL kappa region comprising an amino acid sequence of SEQ ID NO:2, and wherein the region derived from the CL region comprises an amino acid sequence having at least 70%, 75%, 80%, 85%, 90% or 95% identity to SEQ ID NO:2.

10. The binding molecule of claim 9, wherein:
(i) the antigen binding loop at the CD loop region of the CH1 region replaces the amino acid residues TSG of the CD loop of the human IgG1 CH1 region; and/or
(ii) the antigen binding loop at the DE loop region of the CH1 region replaces the amino acid residues QSS of the DE loop of the human IgG1 CH1 region.

11. The binding molecule of claim 9, wherein:
(i) the antigen binding loop at the CD loop region of the CL region replaces the amino acid residues SGNS of the CD loop of the human CL kappa region; and/or
(ii) the antigen binding loop at the DE loop region of the CL region replaces the amino acid residues SKD of the DE loop of the human CL kappa region.

12. The binding molecule of claim 1, wherein each of the one or more antigen binding loop(s) comprises 7 to 15 amino acid residues.

13. The binding molecule of claim 2, wherein the VH region and the VL region bind to a first antigen; and the region derived from the CH1 region and/or the region derived from the CL region bind to a second antigen.

14. The binding molecule of claim 13, wherein:
(i) the first antigen and the second antigen are the same antigen; or
(ii) the first antigen and the second antigen are two different antigens.

15. A nucleic acid encoding the binding molecule of claim 1.

16. A vector comprising the nucleic acid of claim 15.

17. A method of making a binding molecule, comprising expressing a polynucleotide encoding the binding molecule of claim 1 in a host cell.

18. A pharmaceutical composition comprising (a) the binding molecule of claim 1, and (b) a pharmaceutically acceptable excipient.

19. A method of treating a disease or disorder in a subject, comprising administering to the subject the binding molecule of claim 1 and/or the nucleic acid of claim 15, optionally wherein the disease or disorder is associated with the first antigen and/or the second antigen.

20. Use of the binding molecule of claim 1 and/or the nucleic acid of claim 15 for the manufacture of a medicament for the treatment of a disease or disorder.

21. A constant region library (CRL) comprising a population of binding molecules, wherein each of the binding molecules is the binding molecule of claim 1, wherein the population of the binding molecules comprise diverse amino acid sequences in the region derived from the CH1 region and/or the region derived from the CL region.

22. A constant region library (CRL) comprising a population of molecules each comprising a region derived from a CH1 region and/or a region derived from a CL region of an antibody, wherein the population of the molecules comprise diverse amino acid sequences in the region derived from the CH1 region and/or the region derived from the CL region.

23. The CRL of claim 21, wherein:
(i) the diverse amino acid sequences in the region derived from the CH1 region are at the AB, BC, CD, DE, EF, and/or FG loop regions of the CH1 region; and/or
(ii) the diverse amino acid sequences in the region derived from the CL region are at the AB, BC, CD, DE, EF, and/or FG loop regions of the CL region.

24. The CRL of claim 21, wherein the population of the molecules comprise diverse amino acid sequences:
(i) in one or two loop region(s) in the region derived from the CH1 region;
(ii) in one or two loop region(s) in the region derived from the CL region;
(iii) at the CD loop region of the CH1 region;
(iv) at the DE loop region of the CH1 region;
(v) at the CD loop region and the DE loop region of the CH1 region;
(vi) at the CD loop region of the CL region;
(vii) at the DE loop region of the CL region;
(viii) at the CD loop region and the DE loop region of the CL region;
(ix) at the CD loop region of the CH1 region; and the population of the molecules comprise diverse amino acid sequences at the CD loop region of the CL region;
(x) at the CD loop region of the CH1 region; and the population of the molecules comprise diverse amino acid sequences at the DE loop region of the CL region;
(xi) at the CD loop region of the CH1 region; and the population of the molecules comprise diverse amino acid sequences at the CD loop region and the DE loop region of the CL region;
(xii) at the DE loop region of the CH1 region; and the population of the molecules comprise diverse amino acid sequences at the CD loop region of the CL region;
(xiii) at the DE loop region of the CH1 region; and the population of the molecules comprise diverse amino acid sequences at the DE loop region of the CL region;
(xiv) at the DE loop region of the CH1 region; and the population of the molecules comprise diverse amino acid sequences at the CD loop region and the DE loop region of the CL region;
(xv) at the CD loop region of the CH1 region and the DE loop region of the CH1 region; and the population of the molecules comprise diverse amino acid sequences at the CD loop region of the CL region;
(xvi) at the CD loop region and the DE loop region of the CH1 region; and the population of the molecules comprise diverse amino acid sequences at the DE loop region of the CL region; or
(xvii) at the CD loop region and the DE loop region of the CH1 region; and the population of the molecules comprise diverse amino acid sequences at the CD loop region and the DE loop region of the CL region.

25. The CRL of claim 21, wherein:
(i) the region derived from the CH1 region is a region derived from a human IgG1 CH1 region comprising an amino acid sequence of SEQ ID NO: 1, and wherein the region derived from the CH1 region comprises an amino acid sequence having at least 70%, 75%, 80%, 85%, 90% or 95% identity to SEQ ID NO: 1; and/or
(ii) the region derived from the CL region is a region derived from a human CL kappa region comprising an amino acid sequence of SEQ ID NO: 2, and wherein the region derived from the CL region comprises an amino acid sequence having at least 70%, 75%, 80%, 85%, 90% or 95% identity to SEQ ID NO: 2.

26. The CRL of claim 25, wherein:
(i) the amino acid residues TSG of the CD loop of the human IgG1 CH1 region are replaced with diverse amino acid sequences in the molecules in the CRL; or
(ii) the amino acid residues QSS of the DE loop of the human IgG1 CH1 region are replaced with diverse amino acid sequences in the molecules in the CRL.

27. The CRL of claim 25, wherein:
(i) the amino acid residues SGNS of the CD loop of the human CL kappa region are replaced with diverse amino acid sequences in the molecules in the CRL; or
(ii) the amino acid residues SKD of the DE loop of the human CL kappa region are replaced with diverse amino acid sequences in the molecules in the CRL.

28. The CRL of claim 21, wherein:
(i) the diverse amino acid sequences comprise 7 to 15 amino acid residues;
(ii) each of the molecules further comprise a VH region and a VL region;
(iii) the binding molecules or the molecules are Fab fragments;
(iv) the diversity of the CRL with one loop region ranges from 10⁷ to 10¹⁶; or
(v) the diversity of the CRL with two loop regions ranges from 10¹⁸ to 10³³.

29. A method for identifying a binding molecule comprising a first binding domain that binds to a first antigen and a second binding domain that binds to a second antigen, comprising screening the CRL of claim 21 for identifying the binding molecule that binds to the second antigen with a higher affinity than a reference level, wherein the first binding domain comprises the VH region and the VL region of an antibody, and wherein the second binding domain comprises an antibody constant region variant.

30. A method of producing a binding molecule comprising a first step for performing a function of identifying an antibody constant region variant capable of binding to an antigen; and a second step of constructing the binding molecule that comprises the antibody constant region variant, optionally wherein the first step comprising screening the CRL of claim 21.

31. A binding molecule identified according to the method of claim 29.

32. A binding molecule produced according to the method of claim 30.

33. A method for treating a disease or disorder in a subject comprising administering to the subject the binding molecule of claim 1.


SEQUENCE LISTING (48 sequences)

SEQ ID NO: 1 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 107 aa, Homo sapiens [sequence omitted]

SEQ ID NO: 3 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 4 - protein, 107 aa, Homo sapiens [sequence omitted]

SEQ ID NO: 5 - protein, 103 aa, Homo sapiens; identical to SEQ ID NO: 1

SEQ ID NO: 6 - protein, 9 aa, Artificial Sequence
  Description of Artificial Sequence Synthetic peptide
SRHHHHHGD

SEQ ID NO: 7 - protein, 8 aa, Artificial Sequence
  Description of Artificial Sequence Synthetic peptide
SRHHHHGD

SEQ ID NO: 8 - protein, 15 aa, Artificial Sequence
  Description of Artificial Sequence Synthetic peptide
RYRAHHNHHDGHHSA

SEQ ID NO: 9 - protein, 13 aa, Artificial Sequence
  Description of Artificial Sequence Synthetic peptide
AHQTDHHHHHQDG

SEQ ID NO: 10 - protein, 11 aa, Artificial Sequence
  Description of Artificial Sequence Synthetic peptide
DGAHHHHHASR

SEQ ID NO: 11 - protein, 7 aa, Artificial Sequence
  Description of Artificial Sequence Synthetic peptide
RHHYRRG

SEQ ID NO: 12 - protein, 9 aa, Artificial Sequence
  Description of Artificial Sequence Synthetic peptide
SLHHHHHGD

SEQ ID NO: 13 - protein, 9 aa, Artificial Sequence
  Description of Artificial Sequence Synthetic peptide
SHHHHHHGD

SEQ ID NO: 14 - protein, 9 aa, Artificial Sequence
  Description of Artificial Sequence Synthetic peptide
SRHHHHHGN

SEQ ID NO: 15 - protein, 24 aa, Artificial Sequence
  Description of Artificial Sequence Synthetic peptide
GALTAGAAGAYQAYPGTARGAGVH

SEQ ID NO: 16 - protein, 19 aa, Artificial Sequence
  Description of Artificial Sequence Synthetic peptide
TAGAAGAYQAYPGTARGAG

SEQ ID NO: 17 - protein, 9 aa, Artificial Sequence
  Description of Artificial Sequence Synthetic peptide
DTSQDGGHT

SEQ ID NO: 18 - protein, 9 aa, Artificial Sequence
  Description of Artificial Sequence Synthetic peptide
GNHHADHGR

SEQ ID NO: 19 - protein, 9 aa, Artificial Sequence
  Description of Artificial Sequence Synthetic peptide
RHEDARLSD

SEQ ID NO: 20 - protein, 9 aa, Artificial Sequence
  Description of Artificial Sequence Synthetic peptide
TDNDAGNHD

SEQ ID NO: 21 - protein, 21 aa, Artificial Sequence
  Description of Artificial Sequence Synthetic peptide
TAGKDDVTGDKKKKNPESGAG

SEQ ID NO: 22 - protein, 21 aa, Artificial Sequence
  Description of Artificial Sequence Synthetic peptide
TAGSRLALSQWSQWQDDWGAG

SEQ ID NO: 23 - protein, 21 aa, Artificial Sequence
  Description of Artificial Sequence Synthetic peptide
TAGYKAWRATRQEKTAWYGAG

SEQ ID NO: 24 - protein, 21 aa, Artificial Sequence
  Description of Artificial Sequence Synthetic peptide
TAGSRAYPDSYSHVKRVAGAG

SEQ ID NO: 25 - protein, 27 aa, Artificial Sequence
  Description of Artificial Sequence Synthetic peptide
EQDSAGASWWWSEAHLWGLTSGADSTY

SEQ ID NO: 26 - protein, 27 aa, Artificial Sequence
  Description of Artificial Sequence Synthetic peptide
EQDSAGRRRGHKHRKRWRSRRGADSTY

SEQ ID NO: 27 - protein, 13 aa, Artificial Sequence
  Description of Artificial Sequence Synthetic peptide
EQDDGGHQYSSTY

SEQ ID NO: 28 - protein, 15 aa, Artificial Sequence
  Description of Artificial Sequence Synthetic peptide
EQDSGGHDYRDSSTY

SEQ ID NO: 29 - protein, 27 aa, Artificial Sequence
  Description of Artificial Sequence Synthetic peptide
EQDSAGAWLHHGDYWGQENPAGADSTY

SEQ ID NO: 30 - protein, 15 aa, Artificial Sequence
  Description of Artificial Sequence Synthetic peptide
EQDNRLQTSNDASTY

SEQ ID NO: 31 - protein, 27 aa, Artificial Sequence
  Description of Artificial Sequence Synthetic peptide
EQDSAGRSQAQNNWDTYGPLSGADSTY

SEQ ID NO: 32 - protein, 15 aa, Artificial Sequence
  Description of Artificial Sequence Synthetic peptide
EQDGTSGRYEQASTY

SEQ ID NO: 33 - protein, 27 aa, Artificial Sequence
  Description of Artificial Sequence Synthetic peptide
EQDSAGWVVSTDYSPQTWSYAGADSTY

SEQ ID NO: 34 - protein, 15 aa, Artificial Sequence
  Description of Artificial Sequence Synthetic peptide
EQDDTYYLAVHGSTY

SEQ ID NO: 35 - protein, 27 aa, Artificial Sequence
  Description of Artificial Sequence Synthetic peptide
EQDSAGKNAVRENAEYLLATSGADSTY

SEQ ID NO: 36 - protein, 27 aa, Artificial Sequence
  Description of Artificial Sequence Synthetic peptide
EQDSAGSQSSDPGSNQESHQSGADSTY

SEQ ID NO: 37 - protein, 27 aa, Artificial Sequence
  Description of Artificial Sequence Synthetic peptide
EQDSAGWASPRLAYPDTVPVAGADSTY

SEQ ID NO: 38 - protein, 27 aa, Artificial Sequence
  Description of Artificial Sequence Synthetic peptide
EQDSAGANRLKGQKQPNDKQSGADSTY

SEQ ID NO: 39 - protein, 27 aa, Artificial Sequence
  Description of Artificial Sequence Synthetic peptide
EQDSAGANSPLQNRREQWGRKGADSTY

SEQ ID NO: 40 - protein, 27 aa, Artificial Sequence
  Description of Artificial Sequence Synthetic peptide
EQDSAGAPWYVHNYTRWRKNWGADSTY

SEQ ID NO: 41 - protein, 27 aa, Artificial Sequence
  Description of Artificial Sequence Synthetic peptide
EQDSAGWKQWWGLWWHPVQTSGADSTY

SEQ ID NO: 42 - protein, 15 aa, Artificial Sequence
  Description of Artificial Sequence Synthetic peptide
EQDSGDTNNYTRSTY

SEQ ID NO: 43 - protein, 17 aa, Artificial Sequence
  Description of Artificial Sequence Synthetic peptide
AGAAGAYQAYPGTARGA

SEQ ID NO: 44 - protein, 19 aa, Artificial Sequence
  Description of Artificial Sequence Synthetic peptide
AGSRAYPDSYSHVKRVAGA

SEQ ID NO: 45 - protein, 19 aa, Artificial Sequence
  Description of Artificial Sequence Synthetic peptide
AGASWWWSEAHLWGLTSGA

SEQ ID NO: 46 - protein, 19 aa, Artificial Sequence
  Description of Artificial Sequence Synthetic peptide
AGWASPRLAYPDTVPVAGA

SEQ ID NO: 47 - protein, 4 aa, Artificial Sequence
  Description of Artificial Sequence Synthetic peptide
GALT

SEQ ID NO: 48 - protein, 4 aa, Artificial Sequence
  Description of Artificial Sequence Synthetic peptide
SGVH